(19) United States
(12) Patent Application Publication
(10) Pub. No.: US 2010/0099109 A1
(43) Pub. Date: Apr. 22, 2010

(54) Methods for Analyzing Drug Response

(75) Inventors: Wendy J. Fantl, San Francisco, CA (US); David Rosen, Mountain View, CA (US)

Correspondence Address: WILSON, SONSINI, GOODRICH & ROSATI / NODALITY, INC, 650 Page Mill Road, Palo Alto, CA, 94304-1050, US

(73) Assignee: NODALITY, INC., A DELAWARE CORPORATION

(21) Appl. No.: 12/581,536

(22) Filed: Oct. 19, 2009

Related U.S. Application Data
(60) Provisional application No. 61/106,462, filed on Oct. 17, 2008.
(60) Provisional application No. 61/156,754, filed on Mar. 2, 2009.
(60) Provisional application No. 61/186,619, filed on Jun. 12, 2009.

(51) Int. Cl. C12Q 1/68; G01N 33/53

(52) U.S. Cl. 435/6; 435/7.21

(57) ABSTRACT

The present invention provides methods, instruments, reagents, kits and the biology involved in analyzing drug response. An embodiment of the present invention provides an approach for the characterization a plurality of pathways in single cells. This approach permits the rapid detection of heterogeneity in a complex cell population based on activation states of cellular molecules such as proteins, expression markers and other criteria, and the identification of cellular subsets that exhibit correlated changes in activation within the cell population. Some of these categories include redox potential, ITIM phosphorylation, intracellular pH and other categories allows for characterization of such pathways and cell populations. Also, the present analysis is useful for the analysis of the effect of compounds on potential target cells.

### CROSS-REFERENCE

This application claims the benefit of U.S. Provisional Application No. 61/106,462 filed Oct. 17, 2008, U.S. Provisional Application No. 61/156,754 filed Mar. 2, 2009, and U.S. Provisional Application No. 61/186,619 filed Jun. 12, 2009, which applications are incorporated herein by reference in their entirety.

### BACKGROUND OF THE INVENTION

Many conditions are characterized by disruptions in cellular pathways that lead, for example, to aberrant control of cellular processes, with uncontrolled growth and increased cell survival. These disruptions are often caused by changes in the activity of molecules participating in cellular pathways. For example, alterations in specific signaling pathways have been described for many cancers. This disruptions and/or alterations in cellular pathways may contribute to the ability of a cell to respond to treatment.

Accordingly, there is a need to look at cell populations to determine what cell signaling events may contribute to their responses to compounds.

### SUMMARY OF THE INVENTION

In some embodiments, the invention provides methods, compositions and devices for analyzing the effect of a therapeutic agent on a cell. In some embodiments, the invention provides methods of classification, diagnosis, prognosis and/or prediction of an outcome of a condition in an individual, the methods comprising the steps of: (i) contacting a cell population from the individual with a DNA damage or apoptosis inducing therapeutic agent, where the cell population comprises one or more cells associated with a condition, and where the agent is used to treat the condition; (ii) determining an activation level of at least one activatable element within a DNA damage pathway and an activation level of at least one activatable element within an apoptosis pathway in one or more cells from the cell population; and (iii) making a decision regarding classification, diagnosis, prognosis and/or prediction of an outcome of the condition in the individual, where the decision is based on the activation levels of the at least one activatable element within the DNA damage pathway and the at least one activatable element within the apoptosis pathway.

In some embodiments, the cell population is a hematopoietic cell population. In some embodiments, the hematopoietic cell population is selected from the group consisting of pluripotent hematopoietic stem cells, T-lymphocyte lineage progenitor or derived cells, B-lymphocyte lineage progenitor or derived cells, granulocyte lineage progenitor or derived cells, monocyte lineage progenitor or derived cells, megakaryocyte lineage progenitor or derived cells, and erythroid lineage progenitor or derived cells. In some embodiments, the cell population might comprise one or more cells that might be resistant to the DNA damage or apoptosis inducing therapeutic agent. In some embodiments, the condition is acute leukemia, myelodysplastic syndrome or myeloproliferative neoplasms. In some embodiments, the acute leukemia is acute myeloid leukemia.

In some embodiments, the DNA damage or apoptosis inducing therapeutic agent is selected from the group consisting of Staurosporine, Etoposide, Mylotarg, Daunorubicin, Idarubicin and analogs (idarubicin, epirubicin), Ara-C, Vidaza, Mitoxantrone, Clofarabine, Cladribine, Dacogen, HydroxyUrea, Zolinza, Rituxan, Fludarabine, Floxuridine, 5-FU, Gemcitabine, Cisplatin, ifosfamide, alkylating agents, nucleoside analogs, mechlorethamine and other nitrogen mustards, mercaptopurine, teniposide, Thioguanine, topotecan, and troxacitabine. In some embodiments, the DNA damage or apoptosis inducing agent is mylotarg.

In some embodiments, the at least one activatable element within the DNA Damage pathway is selected from the group consisting of p-Chk1, p-Chk2, p-53, p-ATM, and p-H2AX. In some embodiments, the activatable element within the apoptosis pathway is selected from the group consisting of Cleaved PARP, Cleaved Caspase 3, Cleaved Caspase 8, BAX, Bak, and Cytochrome C.

In some embodiments, the methods further comprise determining a functional state of the apoptosis pathway or the DNA damage pathway, where the functional state is based on the activation levels of the activatable elements. In some embodiments, determining the functional state further comprises a prediction of the outcome of the condition to treatment with the DNA damage or apoptosis inducing therapeutic agent, where the individual is predicted to respond to treatment if both the apoptosis and DNA damage pathways are functional the individual can respond to treatment, where the individual is predicted to respond to treatment if the DNA damage pathway is not functional but the apoptosis pathway is functional, where the individual is predicted not to respond to treatment if the DNA damage pathway is functional but the apoptosis pathway is not functional, and where the individual is predicted not to respond to treatment if both the apoptosis and DNA damage pathways are not functional. In some embodiments, the determination guides selection of a therapeutic treatment for the individual.

In some embodiments, the methods further comprise determining the activation level of at least one activatable element within a cell cycle pathway. In some embodiments, the at least one activatable element within a cell cycle pathway is selected from the group consisting of Cdc25, p-p53, cCdk1, CyclinB1, p16, p21, p-Histone H3 and Gadd45.

In some embodiments, the methods further comprise contacting the cell population comprising one or more cells associated with the condition from an individual with an additional modulator and characterizing an additional pathway by determining the activation level of at least one activatable element within the additional pathway. In some embodiments, the additional pathway is selected from the group consisting of drug conversion into an active agent, internal cellular pH, redox potential environment, phosphorylation state of ITIM; drug activation; and signaling pathways. In some embodiments, the additional pathway is selected from the group consisting of Jak/Stat, PI3K/Akt, and MAPK pathways. In some embodiments, the activatable element within the PI3K/AKT or MAPK pathways is selected from the group consisting of Akt, p-ERK, p-SyK, p38 and pS6 and the modulator is selected from the group consisting of FLT3L, SCF, G-CSF, GM-CSF, SCF, SDF1a, LPS, PMA, and Thapsigargin. In some embodiments, the activatable element within the PI3K/AKT or MAPK pathways is selected from the group consisting of p-Akt, p-ERK, and pS6 and the modulator is selected from the group consisting of SCF, and PMA. In some embodiments, the activatable element within the STAT pathway is selected from the group consisting of p-Stat3, p-Stat5, p-Stat1, and p-Stat6 and the modulator is selected from the group consisting of IFNg, IFNa, IL-27, IL-3, IL-6, IL-10, GM-CSF and G-CSF. In some embodiments, the activatable element within the STAT pathway is p-Stat1 and the modulator is IL-6.

In some embodiments, the methods further comprise determining the presence or absence of one or more cell surface markers, intracellular markers, or combination thereof. In some embodiments, the cell surface markers and the intracellular markers are independently selected from the group consisting of proteins, carbohydrates, lipids, nucleic acids and metabolites. In some embodiments, the determining of the presence or absence of one or more cell surface markers or intracellular markers comprises determining the presence or absence of an epitope in both activated and non-activated forms of the cell surface markers or the intracellular markers. In some embodiments, the classification, diagnosis, prognosis and/or prediction of outcome of the condition in an individual is based on both the activation levels of the activatable element and the presence or absence of the one or more cell surface markers, intracellular markers, or combination thereof. In some embodiments, the activation level is determined by a process comprising the binding of a binding element which is specific to a particular activation state of the particular activatable element. In some embodiments, the binding element comprises an antibody, recombinant protein, or fluorescent dye. In some embodiments, the step of determining the activation level comprises the use of flow cytometry, immunofluorescence, confocal microscopy, immunohistochemistry, immunoelectronmicroscopy, nucleic acid amplification, gene array, protein array, mass spectrometry, patch clamp, 2-dimensional gel electrophoresis, differential display gel electrophoresis, microsphere-based multiplex protein assays, ELISA, and label-free cellular assays to determine the activation level of one or more intracellular activatable element in single cells.

In some embodiments, the invention provides methods of classification, diagnosis, prognosis and/or prediction of an outcome of a condition in an individual, the methods comprising the steps of: (i) subjecting a cell population from the individual to a therapeutic agent, where the therapeutic agent is used to treat cancer, and where the cell population comprises one or more cells associated with a condition; (ii) determining an activation level of at least one activatable element within a first pathway and an activation level of at least one activatable element within a second pathway in one or more cells from the cell population; (iii) determining the expression and/or function of a drug transporter in the cells or separate cells from the cell population not subjected to the therapeutic agent; and (iv) making a decision regarding classification, diagnosis, prognosis of and/or prediction of an outcome of the condition in the individual, where the decision is based on the activation levels of the at least one activatable element within the first pathway, the activation level of the at least one activatable element within the second pathway and the expression and/or function of the drug transporter. In some embodiments, the methods comprise an alternative step comprising determining the effect of inhibiting a drug transporter on a response to the therapeutic agent in the cell population. In some embodiments, the cell population is a hematopoietic cell population. In some embodiments, the hematopoietic cell population is selected from the group consisting of pluripotent hematopoietic stem cells, T-lymphocyte lineage progenitor or derived cells, B-lymphocyte lineage progenitor or derived cells, granulocyte lineage progenitor or derived cells, monocyte lineage progenitor or derived cells, megakaryocyte lineage progenitor or derived cells, and erythroid lineage progenitor or derived cells. In some embodiments, the cell population might comprise one or more cells that might be resistant to the therapeutic agent. In some embodiments, the condition is acute leukemia, myelodysplastic syndrome or myeloproliferative neoplasms. In some embodiments, the acute leukemia is acute myeloid leukemia.

In some embodiments, the therapeutic agent used to treat cancer is selected from the group consisting of a DNA damaging agent, an apoptosis inducing agent a drug transporter substrate. In some embodiments, the DNA damaging or apoptosis inducing agent is selected from the group consisting of Staurosporine, Etoposide, Mylotarg, Daunorubicin, Idarubicin and analogs (idarubicin, epirubicin), Ara-C, Vidaza, Mitoxantrone, Clofarabine, Cladribine, Dacogen, HydroxyUrea, Zolinza, Rituxan, Fludarabine, Floxuridine, 5-FU, Gemcitabine, Cisplatin, ifosfamide, alkylating agents, nucleoside analogs, mechlorethamine and other nitrogen mustards, mercaptopurine, teniposide, Thioguanine, topotecan, and troxacitabine. In some embodiments, the DNA damaging or Apoptosis inducing agent is mylotarg. In some embodiments, the drug transporter is selected from the group consisting of P-glycoprotein (MDR1), MDR associated protein and breast cancer resistance protein. In some embodiments, the drug transporter is MDR1.

In some embodiments, the first pathway or the second pathway is a DNA damage pathway. In some embodiments, the at least one activatable element within the DNA damage pathway is selected from the group consisting of p-Chk1, p-Chk2, p-p53, p-ATM, and p-H2AX. In some embodiments, the first pathway or the second pathway is an apoptosis pathway. In some embodiments, the activatable element within the apoptosis pathway is selected from the group consisting of Cleaved PARP, Cleaved Caspase 3, Cleaved Caspase 8, BAX, Bak and Cytochrome C. In some embodiments, the first pathway is a DNA damage pathway and the second pathway is as apoptosis pathway. In some embodiments, the methods further comprise determining a functional state of the apoptosis pathway or the DNA damage pathway, where the functional state is based on the activation levels of the activatable elements. In some embodiments, determining a functional state further comprises a prediction of the outcome of the condition to treatment with the therapeutic agent, where the individual is predicted to respond to treatment if both the apoptosis and DNA damage pathways are functional the individual can respond to treatment, where the individual is predicted to respond to treatment if the DNA damage pathway is not functional but the apoptosis pathway is functional, where the individual is predicted not to respond to treatment if the DNA damage pathway is functional but the apoptosis pathway is not functional, and where the individual is predicted not to respond to treatment if both the apoptosis and DNA damage pathways are not functional. In some embodiments, the determination guides selection of a therapeutic treatment for the individual.

In some embodiments, the methods further comprise determining the activation level of at least one activatable element within a cell cycle pathway. In some embodiments, the at least one activatable element within a cell cycle pathway is selected from the group consisting of Cdc25, p-p53, cCdk1, CyclinB1, p16, p21, p-Histone H3 and Gadd45. In some embodiments, the methods further comprise contacting the cell population comprising one or more cells associated with the condition from the individual with an additional modulator and characterizing an additional pathway by determining the activation level of at least one activatable element within the additional pathway. In some embodiments, the additional pathway is selected from the group consisting of drug conversion into an active agent, internal cellular pH, redox potential environment, phosphorylation state of ITIM; drug activation; and signaling pathways. In some embodiments, the additional pathway is selected from the group consisting of Jak/Stat, PI3K/Akt, and MAPK pathways. In some embodiments the activatable element within the PI3K/AKT or MAPK pathways is selected from the group consisting of p-Akt, p-ERK, p38 and pS6 and the modulator is selected from the group consisting of FLT3L, SCF, G-CSF, SCF, GM-CSF, SDF1a, LPS, PMA, and Thapsigargin. In some embodiments, the activatable element within the PI3K/AKT or MAPK pathways is selected from the group consisting of p-Akt, p-ERK, and pS6 and the modulator is selected from the group consisting of SCF, and PMA. In some embodiments, the activatable element within the STAT pathway is selected from the group consisting of p-Stat3, p-Stat5, p-Stat1, and p-Stat6 and the modulator is selected from the group consisting of IFNg, IFNa, IL-27, IL-3, IL-6, IL-10, GM-CSF and G-CSF. In some embodiments, the activatable element within the STAT pathway is p-Stat1 and the modulator is IL-6.

In some embodiments, the methods further comprise determining the presence or absence of one or more cell surface markers, intracellular markers, or combination thereof. In some embodiments, the cell surface markers and the intracellular markers are independently selected from the group consisting of proteins, carbohydrates, lipids, nucleic acids and metabolites. In some embodiments, the determining of the presence or absence of one or more cell surface markers or intracellular markers comprises determining the presence or absence of an epitope in both activated and non-activated forms of the cell surface markers or the intracellular markers. In some embodiments, the classification, diagnosis, prognosis of and/or prediction of outcome of the condition in an individual is based on both the activation levels of the activatable element and the presence or absence of the one or more cell surface markers, intracellular markers, or combination thereof.

In some embodiments, the activation level is determined by a process comprising the binding of a binding element which is specific to a particular activation state of the particular activatable element. In some embodiments, the binding element comprises an antibody, recombinant protein, or fluorescent dye. In some embodiments, the step of determining the activation level comprises the use of flow cytometry, immunofluorescence, confocal microscopy, immunohistochemistry, immunoelectronmicroscopy, nucleic acid amplification, gene array, protein array, mass spectrometry, patch clamp, 2-dimensional gel electrophoresis, differential display gel electrophoresis, microsphere-based multiplex protein assays, ELISA, and label-free cellular assays to determine the activation level of one or more intracellular activatable element in single cells.

In some embodiments, the invention provides methods of classification, diagnosis, prognosis and/or prediction of an outcome of AML in an individual, the method comprising the steps of: (i) providing a population of cells comprising AML cells from an individual; (ii) contacting the cells with therapeutic agent comprising an antibody conjugated to a toxin; (ii) characterizing in individual cells at least three pathways selected from the group consisting of drug conversion into an active agent, cellular redox potential, signaling pathways, DNA damage pathway and apoptosis pathways, where the pathways are characterized by determining the activation level of at least one activatable element within the at least three pathways; and (iv) correlating the classification, diagnosis, prognosis and/or prediction of an outcome of AML in the individual to the characterization of the at least three pathways.

In some embodiments, the methods comprise determining drug binding, a drug transported expression and/or function in the population of cells comprising AML cells from an individual. In some embodiments, the drug transporter is selected from the group consisting of P-glycoprotein (MDR1), MDR associated protein and breast cancer resistance protein. In some embodiments, the drug transporter is MDR1. In some embodiments, the therapeutic agent comprising an antibody conjugated to a toxin is Mylotarg. In some embodiments, the methods comprise determining expression of CD33 and/or ITIM phosphorylation.

In some embodiments, the invention is a method for analyzing the effect of a compound on a cell, comprising; selecting a compound designed to treat cancer cells, wherein resistant cells may arise during a therapeutic treatment using the compound; contacting the cells with the compound; analyzing cellular redox signaling nodes by flow cytometry in which individual cells are simultaneously analyzed for a plurality of nodes; and correlating the results of the analysis with a response to the compound. In another embodiment, the method is for investigating a response to a compound designed to treat cancer in a population of cancer cells; comprising; providing a heterogeneous population of cancer cells having a subpopulation of cells that may be resistant to a compound; contacting the cells with the compound, the compound comprising a binding component and an active component designed to induce cell death or apotosis, wherein the binding component is directed at a cell surface antigen whereby the compound is internalized and cleaved into the binding component and the active component; and analyzing the cellular response to the compound. A further embodiment comprises analyzing at least three of the characteristics below: internal cellular pH; phosphorylation state of the CD33 ITIM; drug transporter function; drug transporter expression; drug conversion into an active agent; signaling pathways in response to modulators such as cytokines, growth factors, chemokines DNA damage repair, and apoptosis. In some embodiments, the cancer compound acts by damaging DNA through mechanisms including, but not limited to intercalation into DNA, inhibiting topoisomerase 1, inhibiting topoisomerase 2, inhibiting DNA or RNA polymerase, inhibiting DNA ligase, inhibiting ribonucleotide reductase, substituting bases, nucleotides or nucleosides or their analogues in nucleic acids, inhibiting DNA damage repair pathways, blocking the mitotic pathway, or the method in which the cancer compound acts by inducing apoptotic or necrotic cell death. In some embodiments, the methods further comprises determining drug binding. In some embodiments, the method as in claim involves determining the activation state of activatable elements, which can be in cell signaling networks, comprising contacting the cell with at least one of: a modulator, an inhibitor, or a compound designed to treat the cancer; and measuring the effect of the modulator, inhibitor or compound on the cell using a flow cytometer; wherein the compound and the modulator are simultaneously contacted with the cell.

In one embodiment of the method, the compound may be an antibody conjugated to a cytotoxic drug, including, but not limited to of Mylotarg. Or the compound may be selected from the group consisting of mitoxantrone, etoposide, daunorubicin, Gleevec, Iressa, AraC, staurosporine, lenalidomide, azacitadine, Clofarabine, Zolinza and decitabine. The method may analyze the activatable elements after perturbing the cell state with a modulator. The method further comprises preparing a signature of a disease state. One embodiment of the method involves determining that the response is a complete response, partial response, no response, resistance/refractory response, progressive disease, stable disease and adverse reaction.

Another embodiment of the present invention is a method for investigating the response to a compound in a population of AML cells having a subpopulation of cells that are resistant to Mylotarg (gemtuzumab ozogamicin), comprising: providing a heterogeneous population of AML cells some of which are resistant to Mylotarg; contacting the cells with Mylotarg; analyzing individual cells using flow cytometry for each of the following categories: drug transporter function, drug transporter expression, drug conversion into an active agent, cellular redox potential, signaling pathways, DNA damage repair, and apoptosis; correlating Mylotarg resistance to the analysis of all of the categories using a quantitative and qualitative analysis of the categories. In some embodiments, the methods further comprises determining drug binding. The correlation may be used to develop drug response signatures for disease cells. The drug transporter system can comprise CNT, ENT, or the ABC transporter family, which includes P-glycoprotein, MDR-associated protein and breast cancer resistance protein. The apoptotic nodes may comprise p53, Bcl-2 family, which includes Bcl-2, Bcl-X_{L}, Mcl-1, Bax, Bak, Noxa, Puma, Bid, Bad and Bim, poly(ADP-ribose) polymerase 1 (PARP), apoptotic protease activating factor (APAF), procaspase 3, 7, 8, or 9, and caspase 3, 7, 8, or 9. The signaling nodes may comprise regulators or signaling molecules in the JAK/STAT, MAPK/ERK, NFkB, WNT, PI3K, PKC, or DNA damage response pathways, for example. Another embodiment may show that the cellular redox nodes comprise levels of glutathione, components of NADPH oxidase system, and concentration of free radicals.

One embodiment of the invention includes a method of profiling a cell population wherein the cells are analyzed by a process comprising: permeabilizing said one or more cells from separate cultures; contacting said permeabilized cells from each of said separate cultures with at least one detectible state-specific binding element for each of said plurality of signaling nodes, wherein said detectible state-specific binding elements are distinguishably labeled; and detecting the presence or absence of binding of each of said distinguishably labeled state-specific binding elements to said plurality of signaling nodes in each of said permeabilized cells from each of said separate cultures; creating a response panel for said cell population comprising said determined signaling node states; and determining a signaling phenotype of said cell population based on said response panel.

Another embodiment of the method comprises the steps of: providing a population of cells that have been gated to separate the cells into discrete subsets; contacting said subsets with a plurality of activation state-specific binding elements, wherein said plurality of activation state-specific binding elements comprise: a first activation state-specific binding element that binds to a first isoform of a first activable protein; and a second activation state-specific binding element that binds to a first isoform of a second activatable protein; and using flow cytometry to detect the presence or absence of binding of said first and second binding elements to determine the activation state of said first and second activatable proteins.

In another embodiment, the modulator can be one or more members of the following classes: a chemical or biological agent which can comprise growth factors, cytokines, chemokines, neurotransmitters, adhesion molecules, hormones, small molecules, inorganic compounds, polynucleotides, antibodies, natural compounds, lectins, lactones, chemotherapeutic agents, biological response modifiers, carbohydrate, proteases, free radicals, cellular or botanical extracts, cellular or glandular secretions, physiologic fluids such as serum, amniotic fluid, or venom; or the modulator can be physical and environmental stimuli which can comprise electromagnetic, ultraviolet, infrared or particulate radiation, redox potential, pH, the presence or absences of nutrients, changes in temperature, changes in oxygen partial pressure, reactive oxygen species, changes in ion concentrations and the application of oxidative stress.

In one embodiment of the invention, a set of at least three response signatures in patient cells treated with the drug Mylotarg can be used to select a therapeutic or combination of therapeutics for the patient. These signatures are bases on (a) DNA damage, which can be measured for example by increases in p-CHK2 and p-H2AX levels, and (b) apoptosis, which can be measured, for example, by increases in cleaved Caspase 3, cleaved PARP, forward and side scatter of light, or viability dye staining. When patient samples exhibit the first signature, in which DNA damage and apoptosis are increased, these patients are likely to respond to Mylotarg, and are good candidates for Mylotarg treatment (See FIG. 2). When patient samples exhibit the second signature, in which a DNA damage response is seen without an increase in apoptosis, these patients are unlikely to respond to Mylotarg alone. The methods of the invention can be used to screen combinations of Mylotarg and other therapeutic agents, for example, pro-apoptotic drugs to select a treatment regimen for these patients (See FIG. 3). When patient samples exhibit the third signature, in which neither a DNA damage nor apoptosis response is seen these patients are unlikely to respond to Mylotarg alone. The lack of response may be due to mechanisms including but not limited to lack of extracellular CD33 binding sites, improper internalization or processing of Mylotarg, an abnormal cellular apoptotic machinery, aberrantly activated cellular survival pathways. The methods of the invention can be used to screen combinations of Mylotarg and other therapies, including but not limited to for example, drug pump inhibitors to select a treatment regimen that allows these patients to respond to Mylotarg (See FIG. 4).

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows that cell lines refractory to Mylotarg have a PMA mediated increase in p-S6

FIG. 2 shows a signature response to Mylotarg in primary AML sample: Mylotarg induces DNA damage response and apoptosis.

FIG. 3 shows a signature response to Mylotarg in primary AML sample: Mylotarg induces DNA damage response, but no apoptosis.

FIG. 4 shows a signature response to Mylotarg in primary AML sample: Mylotarg induces neither DNA damage nor apoptosis responses.

FIG. 5 shows a functional drug transporter assay that detects MDR1 activity in Mylotarg refractory cells.

### DETAILED DESCRIPTION OF THE INVENTION

The present invention incorporates information disclosed in other applications and texts. The following patent and other publications are hereby incorporated by reference in their entireties: Haskell et al, Cancer Treatment, 5^{th} Ed., W.B. Saunders Co., 2001; Alberts et al., Molecular Biology of the Cell, 4^{th} Ed., Garland Science, 2002; Vogelstein and Kinzler, The Genetic Basis of Human Cancer, 2d Ed., McGraw Hill, 2002; Michael, Biochemical Pathways, John Wiley and Sons, 1999; Weinberg, The Biology of Cancer, 2007; Immunobiology, Janeway et al. 7^{th} Ed., Garland Science, Leroith and Bondy, Growth Factors and Cytokines in Health and Disease, A Multi Volume Treatise, Volumes 1A and 1B, Growth Factors, 1996. Other conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3^{rd} Ed., W.H. Freeman Pub., New York, N.Y. and Berg et al. (2002) Biochemistry, 5^{th} Ed., W.H. Freeman Pub., New York, N.Y.; and Sambrook, Fritsche and Maniatis. “Molecular Cloning A laboratory Manual” 3^{rd} Ed. Cold Spring Harbor Press (2001), all of which are herein incorporated in their entirety by reference for all purposes.

Also, patents and applications that are incorporated by reference include U.S. Pat. Nos. 7,381,535; 7,393,656; U.S. patent application Ser. Nos. 10/193,462; 11/655,785; 11/655,789; 11/655,821; 11/338,957, 61/048,886; 61/048,920; 61/048,657; 61/079,766, and 61/085,789.

Some commercial reagents, protocols, software and instruments that are useful in some embodiments of the present invention are available at the Becton Dickinson Website http://www.bdbiosciences.com/features/products/, and the Beckman Coulter website, http://www.beckmancoulter.com/Default.asp?bhfv=7. Relevant articles include High-content single-cell drug screening with phosphospecific flow cytometry, Krutzik et al., Nature Chemical Biology, 23 Dec. 2007; Irish et al., FLt3 ligand Y591 duplication and Bcl-2 over expression are detected in acute myeloid leukemia cells with high levels of phosphorylated wild-type p53, Neoplasia, 2007, Irish et al. Mapping normal and cancer cell signaling networks: towards single-cell proteomics, Nature, Vol. 6 146-155, 2006; and Irish et al., Single cell profiling of potentiated phospho-protein networks in cancer cells, Cell, Vol. 118, 1-20 Jul. 23, 2004; Schulz, K. R., et al., Single-cell phospho-protein analysis by flow cytometry, Curr Protoc Immunol, 2007, 78:8 8.17.1-20; Krutzik, P. O., et al., Coordinate analysis of murine immune cell surface markers and intracellular phosphoproteins by flow cytometry, J. Immunol. 2005 Aug. 15; 175(4):2357-65; Krutzik, P. O., et al., Characterization of the murine immunological signaling network with phosphospecific flow cytometry, J Immunol 2005 Aug. 15; 175(4):2366-73; Shulz et al., Current Protocols in Immunology 2007, 78:8.17.1-20; Stelzer et al. Use of Multiparameter Flow Cytometry and Immunophenotyping for the Diagnosis and Classification of Acute Myeloid Leukemia, Immunophenotyping, Wiley, 2000; and Krutzik, P. O. and Nolan, G. P., Intracellular phospho-protein staining techniques for flow cytometry: monitoring single cell signaling events, Cytometry A. 2003 October; 55(2):61-70; Hanahan D., Weinberg, The Hallmarks of Cancer, CELL, 2000 Jan. 7; 100(1) 57-70; Krutzik et al, High content single cell drug screening with phophosphospecific flow cytometry, Nat Chem. Biol. 2008 February; 4(2):132-42. Experimental and process protocols and other helpful information can be found at http:/proteomices.stanford.edu. The articles and other references cited below are also incorporated by reference in their entireties for all purposes.

In some embodiments, the present invention involves the classification, diagnosis, prognosis of disease or prediction of outcome after administering a therapeutic agent to treat a condition; exemplary conditions include cancers such as AML, MDS and MPN. In other embodiments, the invention involves monitoring and predicting the outcome of a condition after treatment with a therapeutic agent. In other embodiments, the invention involves selection of a treatment for a condition. In other embodiments, the invention involves drug screening using some of the methods described herein, to determine which drug or combination of drugs may be useful in a particular condition. In other embodiments, the invention involves the identification of new druggable targets, that can be used alone or in combination with other treatments. In addition, the invention allows the selection of patients for specific target therapies. The invention allows for delineation of subpopulations of cells associated with a condition that are differentially susceptible to drugs or drug combinations. In another embodiment, the invention allows to demarkate subpopulations of cells associated with a condition that have different genetic subclone origins. In another embodiment, the invention provides for the identification of a cell type, that in combination with other cell type(s), provide ratiometric or metrics that singly or coordinately allow for surrogate identification of subpopulations of cells associated with a disease, diagnosis, prognosis, disease stage of the individual from which the cells were derived, response to treatment, monitoring and predicting outcome of disease.

Another embodiment involves the analysis of DNA damage pathways, apoptosis pathways, cell cycle pathways, drug transporter function, drug transporter expression, drug conversion into an active agent, internal cellular pH, redox potential environment, phosphorylation state of ITIM; drug activation; and signaling pathways for cytokines and growth factors. In some embodiments, the methods further comprises determining drug binding. In performing these processes, one preferred analysis method involves looking at cell signals and/or expression markers. One embodiment of cell signal analysis involves the analysis of phosphorylated proteins and the use of flow cytometers in that analysis. In one embodiment, a signal transduction-based classification of a condition (e.g. response to treatment) can be performed using clustering of phospho-protein patterns or biosignatures.

In some embodiments, the present invention provides methods for classification, diagnosis, prognosis of a disease, and/or prediction of outcome after administering a therapeutic agent to treat the disease by characterizing a plurality of pathways in a population of cells. In some embodiments, the present invention provides methods for classification, diagnosis, prognosis of a disease and/or prediction of outcome after administering a therapeutic agent to treat the disease by determining a drug transporter expression and/or function. In some embodiments, the present invention provides methods for classification, diagnosis, prognosis of disease and/or prediction of outcome after administering a therapeutic agent to treat the disease by determining a drug transporter expression and/or function and by characterizing one or more pathways in a population of cells. In some embodiments, the therapeutic agent is a therapeutic to treat cancer. In some embodiments, the therapeutic agent is a DNA damaging agent. In some embodiments, the therapeutic agent is an apoptosis and/or cell death inducing agent. In some embodiments, the therapeutic agent is a drug transporter substrate. In some embodiments, a treatment or a combination of treatments is chosen based on the characterization of plurality of pathways in single cells. In some embodiments, characterizing a plurality of pathways in single cells comprises determining whether apoptosis pathways, cell cycle pathways, or DNA damage pathways are functional in an individual in response to a therapeutic agent based on the activation levels of activatable elements within the pathways, where a pathway is functional if the activatable elements within the pathways change their activation state in response to the therapeutic agent. For example, when the apoptosis, cell cycle, signaling, and DNA damage pathways are functional the individual may be able to respond to treatment, and when at least one of the pathways is not functional the individual can not respond to treatment. In some embodiments, if the apoptosis and DNA damage pathways are functional the individual can respond to treatment. In some embodiments, a drug transporter expression and/or function in combination with characterization of one or more pathways is used for classification, diagnosis, prognosis of disease and prediction of outcome after administering a therapeutic agent in an individual. For example, an individual may not respond to treatment to a therapeutic agent if there is efflux of the therapeutic agent from the cell due to a drug transporter activity and due other disruptions in cellular pathways.

The characterization of pathways in conditions such as cancers may show disruptions in cellular pathways that are reflective of the inability of the cancer cells to respond to treatment. These disruptions may indicate increased proliferation, increased survival, evasion of apoptosis, insensitivity to anti-growth signals, efflux of therapeutic agents and other mechanisms, one or more of which could be the cause for the inability of the cancer cells to respond to treatment with a therapeutic agent. In some embodiments, the disruption in these pathways can be revealed by exposing a cell to one or more modulators that mimic one or more environmental cues and/or exposing a cell to a therapeutic agent.

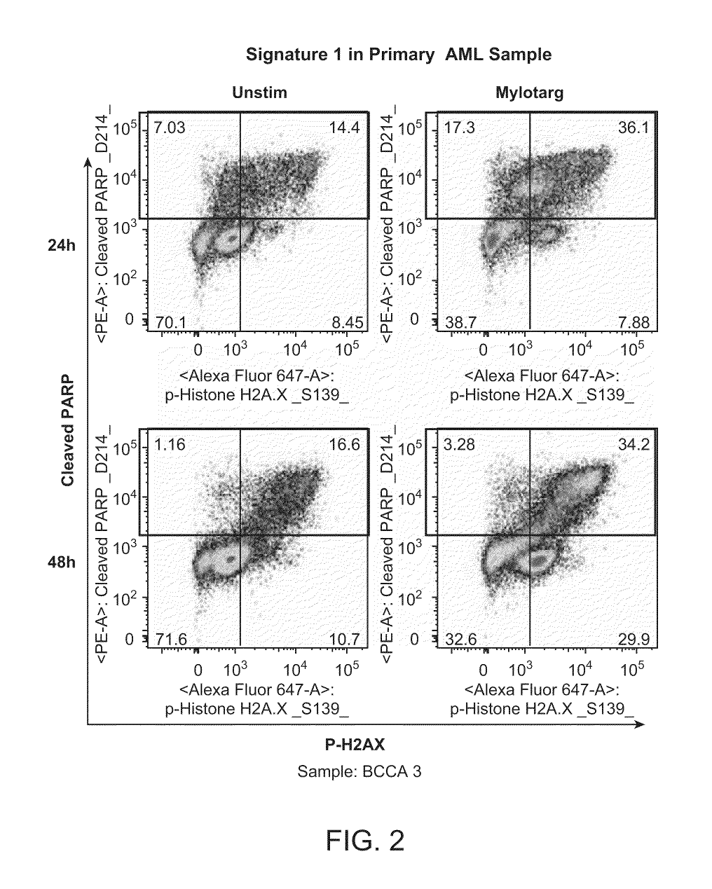

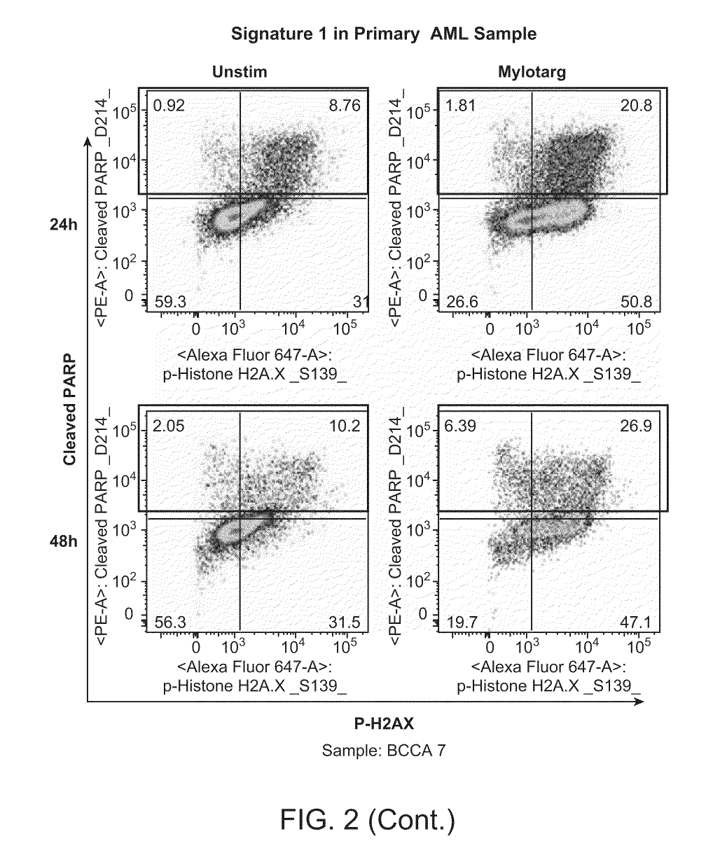

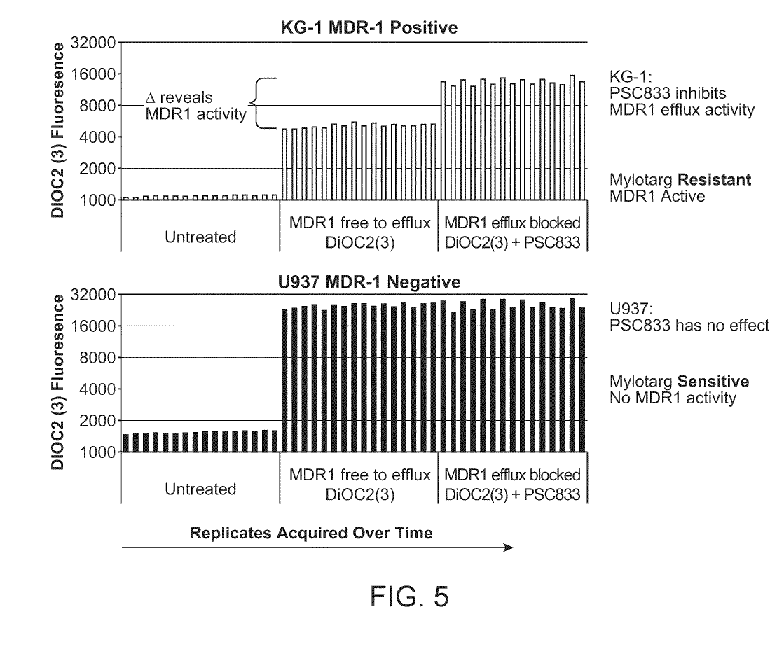

For instance, some of the examples described herein show that responsive cells treated with Mylotarg will undergo cell death through activation of DNA damage and apoptosis pathways (e.g., FIG. 2). However, a non-responsive cell might escape apoptosis through disruption in one or more pathways that allows the cell to survive. Some of the examples described herein show that some non-responsive cells have increased MDR1 function which may cause Mylotarg to be removed from the cells (e.g., FIG. 5). Other examples described herein show that non-responsive cells might also have disruptions in one or more pathways involve in DNA damage response pathway, apoptosis, proliferation, cell cycle progression and cell survival. Table 1 below shows the pathways that can be analyzed to identify disruption in the mechanism of action of Mylotarg.

TABLE 1
Mechanism of Action of Mylotarg

Step in Pathway
CD33 expression
Mylotarg Binding & Internalization
Drug transporter expression
Drug transporter function
Intracellular hydrolysis (pH change)
Redox potential of environment
Signaling pathways (e.g. survival and
proliferation and cell cycle)
DNA damage response
Apoptosis

Aberrant regulations in cellular pathways are revealed, for example, by exposing cells to modulators such as growth factors (e.g. FLT3L or G-CSF). In addition, the aberrant regulation in these pathways can allow for identification of target therapies that will be more effective in a particular patient and can allow for the identification of new druggable targets to which therapies can be used alone or in combination with other treatments. Expression levels of proteins, such as drug transporters and receptors, as a sole measure may not be as informative for disease management as analysis of activatable elements, such as phosphorylated proteins. However, expression information may be useful in combination with the analysis of activatable elements, such as phosphorylated proteins or expression levels of survival proteins including but not limited to members of the surviving family or members of the Bcl-2 family.

The discussion below describes some of the preferred embodiments with respect to particular conditions. However, it should be appreciated that the principles may be useful for the analysis of many other conditions as well.

### Therapeutic Agents

In some embodiments, the present invention provides methods and compositions for classification, diagnosis, prognosis of a condition, and/or prediction outcome after administering a therapeutic agent to treat the condition by characterizing a plurality of pathways in a population of cells. In some embodiments, one or more cells are contacted with therapeutic agent to analyze the response of one or more cells to the therapeutic agent. Responses may include primary refractory behavior (resistance), positive response (full or partial), and other indications such as intensity or duration of response. The results may be useful to determine treatment, understand whether a treatment will work, monitor treatment, modify therapeutic regimens, and to further optimize the selection of therapeutic agents which may be administered as one or a combination of agents. Hence, therapeutic regimens can be individualized and tailored according to the data obtained prior to, and at different times over the course of treatment, thereby providing a regimen that is individually appropriate. The methods of the invention provide tools useful in the treatment with a therapeutic agent of an individual afflicted with a condition, including but not limited to methods for assigning a risk group, methods of predicting an increased risk of relapse, methods of predicting an increased risk of developing secondary complications, methods of choosing a therapy for an individual, methods of predicting duration of response, response to a therapy for an individual, methods of determining the efficacy of a therapy in an individual, and methods of determining the prognosis for an individual. In some embodiments, the therapeutic agent is a therapeutic to treat cancer. In some embodiments, the therapeutic agent is a DNA damaging agent. In some embodiments, the therapeutic agent is an apoptosis and/or cell death inducing agent. In some embodiments, the therapeutic agent is a drug transporter substrate.

In some embodiments, the invention provides methods and compositions for classification, diagnosis, prognosis of a condition, and/or prediction of outcome after administering a therapeutic agent to treat the condition by characterizing a plurality of pathways in a population of cells. In some embodiments, the invention further comprises analyzing a drug transporter expression and/or function. Therapeutic agents to treat cancer include chemotherapeutic agents, angiogenesis inhibitors; biological therapies such as interferons, interleukins, colony-stimulating factors, monoclonal antibodies, vaccines, gene therapy, and nonspecific immunomodulating agents; DNA damaging agents and apoptosis inducing agents. In some embodiments, the therapeutic agent to treat cancer is a DNA damaging agent. In some embodiments, the therapeutic agent to treat cancer is a substrate of a drug transporter such as MDR1.

In some embodiments, the cancer compound acts by damaging DNA through mechanisms including, but not limited to intercalation into DNA, inhibiting topoisomerase 1, inhibiting topoisomerase 2, inhibiting DNA or RNA polymerase, inhibiting DNA ligase, inhibiting ribonucleotide reductase, substituting bases, nucleotides or nucleosides or their analogues in nucleic acids, inhibiting DNA damage repair pathways, blocking the mitotic pathway, or the method in which the cancer compound acts by inducing apoptotic or necrotic cell death.

The therapeutic agent can comprise a binding element and an active component designed to induce cell death or apotosis. In some embodiments, the binding component is directed at a cell surface antigen, whereby the compound may be internalized and cleaved into the binding component and the active component. Active components can be cytotoxic agents or cancer chemotherapeutic agents. Binding agents can be antibodies, antibody fragments, such as single chain fragments, binding peptides, or any compound that can bind a specific cellular element to facilitate entry into the cell to carry the compound that acts on the cell. See Ricart, A D, and Tolcher, A W, Nat Clin Pract Oncol, 2007 April; 4(4):245-55; Singh et al., Curr Med. Chem. 2008; 15(18):1802-26.

Active compounds that can be delivered to the cell using a binding component include agents that induce cell death or apoptosis. These agents may be common cytotoxic agents that are used in cancer chemotherapy, or any other agents that are just generally toxic to cells. Example agents include targeted therapies, such as small molecules directed to biological targets.

Some compounds that contain binding elements attached to elements that can kill or render cells apoptotic are called antibody-drug conjugates. Antibodies are chosen for their ability to selectively target cells with receptors common to tumors. See DiJoseph F, Goad M E, Dougher M M, et al. Potent and specific antitumour efficacy of CMC-544, a CD22-targeted immunoconjugate of calicheamicin, against systemically disseminated B cell lymphoma. Clin Cancer Res. 2004; 10:8620-8629. Upon binding of the antibody—drug conjugate (ADC) to cells, the ADC-receptor complex is internalized into the cell, where the cytotoxic drug is released. Cytotoxic drugs are therefore selected for their potential to induce cell death from within the tumor cell. The molecules that link the antibody to the cytotoxic agent are chosen for their ability to stabilize the conjugate and thus minimize release of the drug before the ADC is internalized into the tumor cell. See Hamann P R. Monoclonal antibody drug conjugates. Expert Opin Ther Patents. 2005; 15:1087-1103; Mandler R, Kobayashi H, Hinson E R, et al. Herceptin-geldanamycin immunoconjugates: pharmacokinetics, biodistribution, and enhanced antitumor activity. Cancer Res. 2004; 64:1460-1467; and Sanderson R J, Hering M A, James S F, et al. In vivo drug-linker stability of an anti-CD30 dipeptide-linked auristatin immunoconjugate. Clin Cancer Res. 2005; 11:843-852.

One example therapeutic agent is trastuzumab-DM1, which is designed to exploit the expression of HER2. This investigational ADC has a proposed dual mechanism of action: anti-HER2 activity and targeted intracellular delivery of DM1, a maytansine derivative that is a potent antimicrotubule agent. By targeting HER2 expression on cancer cells, trastuzumab-DM1 is designed to deliver chemotherapy to tumor cells in a precise manner. Trastuzumab directed cytotoxic therapy: efficacy against HER2-positive trastuzumab-uinsensitive breast cancer models and enhanced response in trastuzumab-sensitive models. 2007 American Association for Cancer Research (AACR) Annual Meeting, Apr. 14-18, 2007. Los Angeles, Calif. Abstract 649. See also, See also Kovtun, Yelena V. et al., Antibody-Drug Conjugates Designed to Eradicate Tumors with Homogeneous and Heterogeneous Expression of the Target Antigen, Cancer Research 66, 3214-3221, Mar. 15, 2006.

Another therapeutic to be analyzed is called Mylotarg. Mylotarg® (gemtuzumab ozogamicin for Injection) is a chemotherapy agent composed of a recombinant humanized IgG4, kappa antibody conjugated with a cytotoxic antitumor antibiotic, calicheamicin, isolated from fermentation of a bacterium, Micromonospora echinospora subsp. calichensis. The antibody portion of Mylotarg binds specifically to the CD33 antigen, a sialic acid-dependent adhesion protein found on the surface of leukemic blasts and immature normal cells of myelomonocytic lineage, but not on normal hematopoietic stem cells

The anti-CD33 hP67.6 antibody is produced by mammalian cell suspension culture using a myeloma NS0 cell line and is purified under conditions which remove or inactivate viruses. Three separate and independent steps in the hP67.6 antibody purification process achieves retrovirus inactivation and removal. These include low pH treatment, DEAE-Sepharose chromatography, and viral filtration. Mylotarg contains amino acid sequences of which approximately 98.3% are of human origin. The constant region and framework regions contain human sequences while the complementarity-determining regions are derived from a murine antibody (p67.6) that binds CD33. This antibody is linked to N-acetyl-gamma calicheamicin via a bifunctional linker. Gemtuzumab ozogamicin has approximately 50% of the antibody loaded with 4-6 moles calicheamicin per mole of antibody. The remaining 50% of the antibody is not linked to the calicheamicin derivative. Gemtuzumab ozogamicin has a molecular weight of 151 to 153 kDa

Mylotarg is a sterile, white, preservative-free lyophilized powder containing 5 mg of drug conjugate (protein equivalent) in an amber vial. The drug product is light sensitive and must be protected from direct and indirect sunlight and unshielded fluorescent light during the preparation and administration of the infusion. The inactive ingredients are: dextran 40; sucrose; sodium chloride; monobasic and dibasic sodium phosphates.

Another therapeutic to be analyzed is called CMC-544. CMC-544 is a CD22 targeted immunoconjugate of calicheamicin and exerts potent cytotoxic effect against CD22+ B cell lymphoma. See: Clin Cancer Res 2006; 12(1), 242-249, Jan. 1, 2006; Clin Cancer Res Vol. 10, 8620-8629, Dec. 15, 2004; and Blood, 1 Mar. 2004, Volume 103, Number 5 1807-1814, incorporated by reference herein it their entirety.

In one embodiment of the method in the therapeutic agent, which may or may not be an antibody conjugated to a cytotoxic drug, including, but not limited to of Mylotarg, zarnestra, sorafenib, gefitnib, tanispomycin, trastuzumab, lepatinib. Or the compound may be selected from the group consisting of mitoxantrone, etoposide, daunorubicin, Idarubicin, idarubicin, epirubicin, Vidaza, Dacogen, Gleevec, Iressa, etoposide, AraC, staurosporine, lenalidomide, azacitadine, HydroxyUrea, decitabine, Zolinza, Rituxan, Fludarabine, Floxuridine, 5-FU, Gemcitabine, Cisplatin, ifosfamide, alkylating agents, nucleoside analogs, mechlorethamine and other nitrogen mustards, mercaptopurine, teniposide, Thioguanine, topotecan, troxacitabine, CSL-360, regrafomib, obatoclax, GDC-0152, GBL-310, ABT-263, phenoxodiol, SGI-1776, AT-101, ABT-869, NRX-5183, AC-220, AS-1411, ARRY-520, AZD-1152, AZD-4877, cediranib (Recentin), L-Vax, Sorafenib, BI-2536, BI-6727, BI-811283, cytarabine, bortezomib, alitretinoin, LOR-2040, annamycin, PR1 peptide antigen vaccine, vorinostat, MG-98, mocetinostat dihydrobromide, ubenimex, elacytarabine, midostaurin, valspodar, cyclosporin A, vatalanib (finasunate), lintuzumab, axitinib, SCH-727965, plitidepsin (Aplidine), arsenic trioxide (Trisenox), fostamatinib (tamatinib fosdium), tretinoin, sapacitabine, cladribine, clofarabine (Clolar/Clofarex/Evoltra), sunitinib (Sutent), oncohist, temozolomide (Temodar), tamibarotene, belinostat, DT388IL-3, amonafide malate (Xanafide), and Volorexin.

### Disease Conditions

The methods of the invention are applicable to any condition in an individual involving, indicated by, and/or arising from, in whole or in part, altered physiological status in a cell. The term “physiological status” includes mechanical, physical, and biochemical functions in a cell. In some embodiments, the physiological status of a cell is determined by measuring characteristics of cellular components of a cellular pathway. Cellular pathways are known in the art. In some embodiments, the cellular pathway is a signaling pathway. Signaling pathways are also known in the art (see, e.g., Hunter T., Cell 100(1): 113-27 (2000); Cell Signaling Technology, Inc., 2002 Catalogue, Pathway Diagrams pgs. 232-253). A condition involving or characterized by altered physiological status may be readily identified, for example, by determining the state in a cell of one or more activatable elements, as taught herein.

In certain embodiments of the invention, the condition is a neoplastic, immunologic or hematopoietic condition. In some embodiments, the neoplastic, immunologic or hematopoietic condition is selected from the group consisting of solid tumors such as head and neck cancer including brain, thyroid cancer, breast cancer, lung cancer, mesothelioma, germ cell tumors, ovarian cancer, liver cancer, gastric carcinoma, colon cancer, prostate cancer, pancreatic cancer, melanoma, bladder cancer, renal cancer, prostate cancer, testicular cancer, cervical cancer, endometrial cancer, myosarcoma, leiomyosarcoma and other soft tissue sarcomas, osteosarcoma, Ewing's sarcoma, retinoblastoma, rhabdomyosarcoma, Wilm's tumor, and neuroblastoma, sepsis, allergic diseases and disorders that include but are not limited to allergic rhinitis, allergic conjunctivitis, allergic asthma, atopic eczema, atopic dermatitis, and food allergy, immunodeficiencies including but not limited to severe combined immunodeficiency (SCID), hypereosiniphic syndrome, chronic granulomatous disease, leukocyte adhesion deficiency I and II, hyper IgE syndrome, Chediak Higashi, neutrophilias, neutropenias, aplasias, agammaglobulinemia, hyper-IgM syndromes, DiGeorge/Velocardial-facial syndromes and Interferon gamma-TH1 pathway defects, autoimmune and immune dysregulation disorders that include but are not limited to rheumatoid arthritis, diabetes, systemic lupus erythematosus, Graves' disease, Graves opthalmopathy, Crohn's disease, multiple sclerosis, psoriasis, systemic sclerosis, goiter and struma lymphomatosa (Hashimoto's thyroiditis, lymphadenoid goiter), alopecia aerata, autoimmune myocarditis, lichen sclerosis, autoimmune uveitis, Addison's disease, atrophic gastritis, myasthenia gravis, idiopathic thrombocytopenic purpura, hemolytic anemia, primary biliary cirrhosis, Wegener's granulomatosis, polyarteritis nodosa, and inflammatory bowel disease, allograft rejection and tissue destructive from allergic reactions to infectious microorganisms or to environmental antigens, and hematopoietic conditions that include but are not limited to Non-Hodgkin Lymphoma, Hodgkin or other lymphomas, acute or chronic leukemias, polycythemias, thrombocythemias, multiple myeloma or plasma cell disorders, e.g., amyloidosis and Waldenstrom's macroglobulinemia, myelodysplastic disorders, myeloproliferative disorders, myelofibroses, or atypical immune lymphoproliferations. In some embodiments, the neoplastic or hematopoietic condition is non-B lineage derived, such as Acute myeloid leukemia (AML), Chronic Myeloid Leukemia (CML), non-B cell Acute lymphocytic leukemia (ALL), non-B cell lymphomas, myelodysplastic disorders, myeloproliferative disorders, myelofibroses, polycythemias, thrombocythemias, or non-B atypical immune lymphoproliferations, Chronic Lymphocytic Leukemia (CLL), B lymphocyte lineage leukemia, B lymphocyte lineage lymphoma, Multiple Myeloma, or plasma cell disorders, e.g., amyloidosis or Waldenstrom's macroglobulinemia.

In some embodiments, the neoplastic or hematopoietic condition is non-B lineage derived. Examples of non-B lineage derived neoplastic or hematopoietic condition include, but are not limited to, Acute myeloid leukemia (AML), Chronic Myeloid Leukemia (CML), non-B cell Acute lymphocytic leukemia (ALL), non-B cell lymphomas, myelodysplastic disorders, myeloproliferative disorders, myelofibroses, polycythemias, thrombocythemias, and non-B atypical immune lymphoproliferations.

In some embodiments, the neoplastic or hematopoietic condition is a B-Cell or B cell lineage derived disorder. Examples of B-Cell or B cell lineage derived neoplastic or hematopoietic condition include but are not limited to Chronic Lymphocytic Leukemia (CLL), B lymphocyte lineage leukemia, B lymphocyte lineage lymphoma, Multiple Myeloma, and plasma cell disorders, including amyloidosis and Waldenstrom's macroglobulinemia.

Other conditions within the scope of the present invention include, but are not limited to, cancers such as gliomas, lung cancer, colon cancer and prostate cancer. Specific signaling pathway alterations have been described for many cancers, including loss of PTEN and resulting activation of Akt signaling in prostate cancer (Whang Y E. Proc Natl Acad Sci USA Apr. 28, 1998; 95(9):5246-50), increased IGF-1 expression in prostate cancer (Schaefer et al., Science Oct. 9, 1998, 282: 199a), EGFR overexpression and resulting ERK activation in glioma cancer (Thomas C Y. Int J Cancer Mar. 10, 2003; 104(1):19-27), expression of HER2 in breast cancers (Menard et al. Oncogene. Sep. 29 2003, 22(42):6570-8), and APC mutation and activated Wnt signaling in colon cancer (Bienz M. Curr Opin Genet Dev 1999 October, 9(5):595-603).

Diseases other than cancer involving altered physiological status are also encompassed by the present invention. For example, it has been shown that diabetes involves underlying signaling changes, namely resistance to insulin and failure to activate downstream signaling through IRS (Burks D J, White M F. Diabetes 2001 February; 50 Suppl 1:S140-5). Similarly, cardiovascular disease has been shown to involve hypertrophy of the cardiac cells involving multiple pathways such as the PKC family (Malhotra A. Mol Cell Biochem 2001 September; 225 (1-):97-107). Inflammatory diseases, such as rheumatoid arthritis, are known to involve the chemokine receptors and disrupted downstream signaling (D'Ambrosio D J Immunol Methods 2003 February; 273 (1-2):3-13). The invention is not limited to diseases presently known to involve altered cellular function, but includes diseases subsequently shown to involve physiological alterations or anomalies

In some embodiments, the present invention is directed to methods for analyzing the effects of a compound designed to treat cancer on one or more cells in a sample derived from an individual having or suspected of having a condition. Example conditions include any solid or hematological malignancy or neoplasm, for example, as well as AML, MDS, or MPN. See U.S. Ser. No. 61/085,789 for a discussion of the above diseases. Further examples include autoimmune, diabetes, cardiovascular, viral and other disease conditions. In some embodiments, the invention allows for identification of prognostically and therapeutically relevant subgroups of the conditions and prediction of the clinical course of an individual.

### Samples and Sampling

The methods involve analysis of one or more samples from an individual. An individual is any multicellular organism; in some embodiments, the individual is an animal, e.g., a mammal. In some embodiments, the individual is a human.

The sample may be any suitable type that allows for the analysis of single cells. Samples may be obtained once or multiple times from an individual. Multiple samples may be obtained from different locations in the individual (e.g., blood samples, bone marrow samples and/or lymph node samples), at different times from the individual (e.g., a series of samples taken to monitor response to treatment or to monitor for return of a pathological condition), or any combination thereof. These and other possible sampling combinations based on the sample type, location and time of sampling allows for the detection of the presence of pre-pathological or pathological cells, the measurement treatment response and also the monitoring for disease.

When samples are obtained as a series, e.g., a series of blood samples obtained after treatment, the samples may be obtained at fixed intervals, at intervals determined by the status of the most recent sample or samples or by other characteristics of the individual, or some combination thereof. For example, samples may be obtained at intervals of approximately 1, 2, 3, or 4 weeks, at intervals of approximately 1, 2, 3, 4, 5, 6, 7, 8, 9, 10, or 11 months, at intervals of approximately 1, 2, 3, 4, 5, or more than 5 years, or some combination thereof. It will be appreciated that an interval may not be exact, according to an individual's availability for sampling and the availability of sampling facilities, thus approximate intervals corresponding to an intended interval scheme are encompassed by the invention. As an example, an individual who has undergone treatment for a cancer may be sampled (e.g., by blood draw) relatively frequently (e.g., every month or every three months) for the first six months to a year after treatment, then, if no abnormality is found, less frequently (e.g., at times between six months and a year) thereafter. If, however, any abnormalities or other circumstances are found in any of the intervening times, or during the sampling, sampling intervals may be modified.

Generally, the most easily obtained samples are fluid samples. Fluid samples include normal and pathologic bodily fluids and aspirates of those fluids. Fluid samples also comprise rinses of organs and cavities (lavage and perfusions). Bodily fluids include whole blood, bone marrow aspirate, synovial fluid, cerebrospinal fluid, saliva, sweat, tears, semen, sputum, mucus, menstrual blood, breast milk, urine, lymphatic fluid, amniotic fluid, placental fluid and effusions such as cardiac effusion, joint effusion, pleural effusion, and peritoneal cavity effusion (ascites). Rinses can be obtained from numerous organs, body cavities, passage ways, ducts and glands. Sites that can be rinsed include lungs (bronchial lavage), stomach (gastric lavage), gastrointestinal track (gastrointestinal lavage), colon (colonic lavage), vagina, bladder (bladder irrigation), breast duct (ductal lavage), oral, nasal, sinus cavities, and peritoneal cavity (peritoneal cavity perfusion). In some embodiments the sample or samples is blood.

Solid tissue samples may also be used, either alone or in conjunction with fluid samples. Solid samples may be derived from individuals by any method known in the art including surgical specimens, biopsies, and tissue scrapings, including cheek scrapings. Surgical specimens include samples obtained during exploratory, cosmetic, reconstructive, or therapeutic surgery. Biopsy specimens can be obtained through numerous methods including bite, brush, cone, core, cytological, aspiration, endoscopic, excisional, exploratory, fine needle aspiration, incisional, percutaneous, punch, stereotactic, and surface biopsy.

In some embodiments, the sample is a blood sample. In some embodiments, the sample is a bone marrow sample. In some embodiments, the sample is a lymph node sample. In some embodiments, the sample is cerebrospinal fluid. In some embodiments, combinations of one or more of a blood, bone marrow, cerebrospinal fluid, and lymph node sample are used.

One or more cells or cell types, or samples containing one or more cells or cell types, can be isolated from body samples. The cells can be separated from body samples by centrifugation, elutriation, density gradient separation, apheresis, affinity selection, panning, FACS, centrifugation with Hypaque, solid supports (magnetic beads, beads in columns, or other surfaces) with attached antibodies, etc. By using antibodies specific for markers identified with particular cell types, a relatively homogeneous population of cells may be obtained. Alternatively, a heterogeneous cell population can be used. Cells can also be separated by using filters. For example, whole blood can also be applied to filters that are engineered to contain pore sizes that select for the desired cell type or class. Rare pathogenic cells can be filtered out of diluted, whole blood following the lysis of red blood cells by using filters with pore sizes between 5 to 10 μm, as disclosed in U.S. patent application Ser. No. 09/790,673. Once a sample is obtained, it can be used directly, frozen, or maintained in appropriate culture medium for short periods of time. Methods to isolate one or more cells for use according to the methods of this invention are performed according to standard techniques and protocols well-established in the art. See also U.S. Ser. Nos. 61/048,886; 61/048,920; and 61/048,657. See also, the commercial products from companies such as BD and BCI as identified above.

See also U.S. Pat. Nos. 7,381,535 and 7,393,656. All of the above patents and applications are incorporated by reference as stated above.

In some embodiments, the cells are cultured post collection in a media suitable for revealing the activation level of an activatable element (e.g. RPMI, DMEM) in the presence, or absence, of serum such as fetal bovine serum, bovine serum, human serum, porcine serum, horse serum, or goat serum. When serum is present in the media it could be present at a level ranging from 0.0001% to 30%.

### Activatable Elements

The methods and compositions of the invention may be employed to examine and profile the status of any activatable element in a cellular pathway, or collections of such activatable elements. Single or multiple distinct pathways may be profiled (sequentially or simultaneously), or subsets of activatable elements within a single pathway or across multiple pathways may be examined (again, sequentially or simultaneously). The cell can be a hematopoietic cell. Examples of hematopoietic cells include but are not limited to pluripotent hematopoietic stem cells, granulocyte lineage progenitor or derived cells, monocyte lineage progenitor or derived cells, macrophage lineage progenitor or derived cells, megakaryocyte lineage progenitor or derived cells and erythroid lineage progenitor or derived cells.

As will be appreciated by those in the art, a wide variety of activation events can find use in the present invention. In general, the basic requirement is that the activation results in a change in the activatable protein that is detectable by some indication (termed an “activation state indicator”), preferably by altered binding of a labeled binding element or by changes in detectable biological activities (e.g., the activated state has an enzymatic activity which can be measured and compared to a lack of activity in the non-activated state). What is important is to differentiate, using detectable events or moieties, between two or more activation states. However, in other instances an activatable element gets activated by increase expression. Thus, in those instances the increase expression of the activatable element will be measured whether or not there is a moiety between two or more activation states of the cells.

As an illustrative example, and without intending to be limited to any theory, an individual phosphorylatable site on a protein can activate or deactivate the protein. Additionally, phosphorylation of an adapter protein may promote its interaction with other components/proteins of distinct cellular signaling pathways. The terms “on” and “off,” when applied to an activatable element that is a part of a cellular constituent, are used here to describe the state of the activatable element, and not the overall state of the cellular constituent of which it is a part. Typically, a cell possesses a plurality of a particular protein or other constituent with a particular activatable element and this plurality of proteins or constituents usually has some proteins or constituents whose individual activatable element is in the on state and other proteins or constituents whose individual activatable element is in the off state. Since the activation state of each activatable element is measured through the use of a binding element that recognizes a specific activation state, only those activatable elements in the specific activation state recognized by the binding element, representing some fraction of the total number of activatable elements, will be bound by the binding element to generate a measurable signal. The measurable signal corresponding to the summation of individual activatable elements of a particular type that are activated in a single cell is the “activation level” for that activatable element in that cell.

Activation levels for a particular activatable element may vary among individual cells so that when a plurality of cells is analyzed, the activation levels follow a distribution. The distribution may be a normal distribution, also known as a Gaussian distribution, or it may be of another type. Different populations of cells may have different distributions of activation levels that can then serve to distinguish between the populations.

In some embodiments, the basis for classifying cells is that the distribution of activation levels for one or more specific activatable elements will differ among different phenotypes. A certain activation level, or more typically a range of activation levels for one or more activatable elements seen in a cell or a population of cells, is indicative that that cell or population of cells belongs to a distinctive phenotype. Other measurements, such as cellular levels (e.g., expression levels) of biomolecules that may not contain activatable elements, may also be used to classify cells in addition to activation levels of activatable elements; it will be appreciated that these levels also will follow a distribution, similar to activatable elements. Thus, the activation level or levels of one or more activatable elements, optionally in conjunction with levels of one or more levels of biomolecules that may or may not contain activatable elements, of cell or a population of cells may be used to classify a cell or a population of cells into a class. Once the activation level of intracellular activatable elements of individual single cells is known they can be placed into one or more classes, e.g., a class that corresponds to a phenotype. A class encompasses a class of cells wherein every cell has the same or substantially the same known activation level, or range of activation levels, of one or more intracellular activatable elements. For example, if the activation levels of five intracellular activatable elements are analyzed, predefined classes of cells that encompass one or more of the intracellular activatable elements can be constructed based on the activation level, or ranges of the activation levels, of each of these five elements. It is understood that activation levels can exist as a distribution and that an activation level of a particular element used to classify a cell may be a particular point on the distribution but more typically may be a portion of the distribution.

In addition to activation levels of intracellular activatable elements, levels of intracellular or extracellular biomolecules, e.g., proteins, may be used alone or in combination with activation states of activatable elements to classify cells. Further, additional cellular elements, e.g., biomolecules or molecular complexes such as RNA, DNA, carbohydrates, metabolites, and the like, may be used in conjunction with activatable states or expression levels in the classification of cells encompassed here.

In some embodiments, cellular redox signaling nodes are analyzed for a change in activation level. Reactive oxygen species (ROS) are involved in a variety of different cellular processes ranging from apoptosis and necrosis to cell proliferation and carcinogenesis. ROS can modify many intracellular signaling pathways including protein phosphatases, protein kinases, and transcription factors. This activity may indicate that the majority of the effects of ROS are through their actions on signaling pathways rather than via non-specific damage of macromolecules. The exact mechanisms by which redox status induces cells to proliferate or to die, and how oxidative stress can lead to processes evoking tumor formation are still under investigation. See Mates, J M et al., Arch Toxicol. 2008 May:82(5):271-2; Galaris D., et al., Cancer Lett. 2008 Jul. 18; 266(1)21-9.

Under normal physiological conditions, a balance exists between oxidants and anti-oxidants in a redox homeostasis. Severe disturbance of this homeostasis causes the accumulation of high levels of reactive oxygen species (ROS). ROS are derived from the reduction of molecular oxygen to generate superoxide which then is converted to other ROS species. ROS are produced primarily by three sources within the cell. The first and a major site of ROS generation is the mitochondrial electron transport chain where electrons escaping from their transport complexes react with oxygen to form superoxide. A second major source of ROS production are from the NADPH oxidase (Nox) complexes, which were originally identified in phagocytes as a key component of the human innate host defense. Subsequently Nox complexes were found in a wide variety of non-phagocytic cells and tissues and contribute to signal transduction, cell proliferation and apoptosis with roles in many physiological processes. Nox consists of membrane-bound subunits that need to interact with cytoplasmic regulatory subunits including the small GTPase Rac in order to become active and produce ROS (Ushio-Fukai and Nakamura, Cancer Lett. (2008) 266 p37). There exists a family of Nox proteins and some of the family members are increased in cancer. The third source of ROS production is generated from other enzymes including xanthine oxidase, cyclooxygenases, lipoxygenases, myeloperoxidase, heme oxidase and cytochrome P450-based enzymes (Kuo., Antioxidants and Redox signaling (2009) 11 p 1). Cytokine growth factor and death receptor signaling can also lead to the production of ROS that function as second messengers playing an important role in signal transduction pathways. For example generation of peroxide transiently inhibits phosphatase activity in a variety kinase cascades (Morgan et al., Cell Research (2008) 18 p 343, Bindoli et al., Antioxidants and Redox Signaling (2008) 10 p 1549.).

As mentioned above, ROS can act as second messengers at submicromolar concentrations and when endogenously elevated they are reduced by anti-oxidants generated by enzymes, such as superoxide dismutase, glutathione peroxidase, catalase, thioredoxin reductase and glutathione S-transferase. Although these anti-oxidant enzymatic systems are considered the most specific and efficient modulators of cellular redox state, several other low molecular weight anti-oxidant states also exist. In particular the tripeptide, γ-glutamylcysteinylglycine (glutathione) exists at milli-molar concentrations inside the cell and is capable of reducing peroxide, lipid peroxides as well as protein disulfide bonds. By acting as an electron donor, glutathione itself gets oxidized to GSSH, and becomes the substrate for glutathione reductase that maintains it in its reduced form GSH. The ratio of reduced to oxidized glutathione is a measure of ROS in the cell. Further, glutathione reductase is constitutively active and induced upon oxidative stress.

In cancer, the intracellular redox potential can have a profound effect on the efficacy of therapeutic agents either through modulating drug transporter function or through changing the oxidation state and therefore activity of the therapeutic agent itself or through modulating drug transporter function such that agents will be extruded from the cell (Kuo, Antioxidants and Redox signaling (2009) 11 p 1, Karihatala et al., (2007) APMIS 115 p81). As an example, Mylotarg, also called Gemtuzumab ozogamicin, consists of a humanized CD33 antibody conjugated to a DNA damaging agent, N-acetyl calicheamicin 1,2 dimethyl hydrazine dichloride. Once internalized the calicheamicin is released from the CD33 antibody through acid hydrolysis and in order for it to be active it needs to be reduced by glutathione. Thus, measuring the intracellular redox state could allow a prediction to be made of how cells will respond to Mylotarg. Another example in which the intracellular redox state plays a role in drug efficacy is for treatment of acute promyelocytic leukemia with arsenic trioxide. The proposed mechanism of action is an increase in NADPH oxidase-generated superoxide levels which promote apoptosis (Chou and Dang, Curr. Opin. Hem. (2004) 12 μl).

Reactive oxygen species can be measured. One example technique is by flow cytometry. See Chang et al., Lymphocyte proliferation modulated by glutamine: involved in the endogenous redox reaction; Clin Exp Immunol 1999 September; 117(3): 482-488. Redox potential can be evaluated by means of an ROS indicator, one example being 2′,7′-dichlorofluorescein-diacetate (DCFH-DA) which is added to the cells at an exemplary time and temperature, such as 37° C. for 15 minutes. DCF peroxidation can be measured using flow cytometry. See Yang K D, Shaio M F. Hydroxyl radicals as an early signal involved in phorbol ester-induced monocyte differentiation of HL60 cells. Biochem Biophys Res Commun. 1994; 200:1650-7 and Wang J F, Jerrells T R, Spitzer J J. Decreased production of reactive oxygen intermediates is an early event during in vitro apoptosis of rat thymocytes. Free Radic Biol Med. 1996; 20:533-42.

Other exemplary fluorescent dyes, include but are not limited to 2-(6-(4′-hydroxy)phenoxy-3H-xanthen-3-on-9-yl)benzoic acid (HPF) and 2-(6-(4′-amino)phenoxy-3H-xanthen-3-on-9-yl)benzoic acid (APF) which both detect ROS species (Setsukinai et al., J. Biol. Chem. (2003) 278 p3170). Other fluorescent probes are derivatives of reduced fluorescein and calcein which are cell-permeant indicators for ROS. Chemically reduced and acetylated forms of, 2′,7′ dichlorofluorescein (DCF) and calcein are non-fluorescent until their acetate groups are removed by intracellular esterases (Molecular probes). Oxidation of what is now a charged form of the dye is mediated by intracellular ROS. This causes the dye to become fluorescent and the amount of fluorescence will be directly related to the intracellular ROS concentration. As an alternative to monitoring ROS levels, since glutathione levels profoundly influence the redox status, the use of ThiolTracker™ Violet can be used to its monitor levels (Molecular Probes).

In some embodiments, other characteristics that affect the status of a cellular constituent may also be used to classify a cell. Examples include the translocation of biomolecules or changes in their turnover rates and the formation and disassociation of complexes of biomolecule. Such complexes can include multi-protein complexes, multi-lipid complexes, homo- or hetero-dimers or oligomers, and combinations thereof. Other characteristics include proteolytic cleavage, e.g. from exposure of a cell to an extracellular protease or from the intracellular proteolytic cleavage of a biomolecule.

In some embodiments, cellular pH is analyzed. See June, C H and Moore, and J S, Curr Protoc Immulon, 2004 December; Chapter 5:Unit 5.5; Leyval, D et al., Flow cytometry for the intracellular pH measurement of glutamate producing Corynebacterium glutamicum, Journal of Microbiological Methods, Volume 29, Issue 2, 1 May 1997, Pages 121-127; Weider, E D, et al., Measurement of intracellular pH using flow cytometry with carboxy-SNARF-1. Cytometry, 1993 November; 14(8):916-21; and Valli, M, et al., Intracellular pH Distribution in Saccharomyces cerevisiae Cell Populations, Analyzed by Flow Cytometry, Applied and Environmental Microbiology, March 2005, p. 1515-1521, Vol. 71, No. 3.

In some embodiments, the activatable element is the phosphorylation of immunoreceptor tyrosine-based inhibitory motif (ITIM). An immunoreceptor tyrosine-based inhibition motif (ITIM), is a conserved sequence of amino acids (S/I/V/LxYxxI/V/L) that is found in the cytoplasmic tails of many inhibitory receptors of the immune system. After ITIM-possessing inhibitory receptors interact with their ligand, their ITIM motif becomes phosphorylated by enzymes of the Src family of kinases, allowing them to recruit other enzymes such as the phosphotyrosine phosphatases SHP-1 and SHP-2, or inositol-phosphatases called SHIPs. These phosphatases can decrease or increase the activation of molecules involved in cell signaling. See Barrow A, Trowsdale J (2006). “You say ITAM and I say ITIM, let's call the whole thing off: the ambiguity of immunoreceptor signalling”. Eur J Immunol 36 (7): 1646-53. When phosphorylated, these phospho-tyrosine residues provide docking sites for the Shps which may result in transmission of inhibitory or activation signals that effect the signaling of neighboring membrane receptor complexes (Paul et al., Blood (2000 96:483).

ITIMs have been reported to impact the internalization of CD33 binding elements. See Walter, r. et al. ITIM-dependant endocytosis of CD33-related Siglecs: role of intracellular domain, tyrosine phosphorylation, and the tyrosine phosphatases, Shpl and Shp, Journal. Leuk. Bio., 83:January 2008, page 200-211. The authors report that the rate of endocytosis is dependent on the phosphorylation of tyrosine residues within ITIMs residing in the cytoplasmic domain of CD33. They reported that increases in tyrosine phosphorylation mediate increases in the internalization of CD33. They also reported that phosphorylated CD33 binds several SH2 domain containing proteins in an ITIM related manner and only some proteins participate in CD33 internalization. Some tyrosine phosphatases interfere with internalization. See also Walter, R., et al., Phosphorylated ITIMs enable ubiquitylation of an inhibitory cell surface receptor, Traffice 2008:9: 267-279; and Walter, R., et al., Influence of CD33 expression levels and ITIM-dependent internalization on gemtuzumab ozogamicin-induced cytotoxicity, Blood 2005:105:1295-1302.

ITIMs can be analyzed by flow cytometry by the use of an antibody or other binding agent to phosphorylated ITIM. The antibody or other binding agent may be used to monitor patient responsiveness in conjunction with other measurements discussed herein.

Additional elements may also be used to classify a cell, such as the expression level of extracellular or intracellular markers, nuclear antigens, enzymatic activity, protein expression and localization, cell cycle analysis, chromosomal analysis, cell volume, and morphological characteristics like granularity and size of nucleus or other distinguishing characteristics. For example, myeloid cells can be further subdivided based on the expression of surface markers including but not limited to CD45, CD34, CD33, CD11B, CD14.

Alternatively, predefined classes of cells can be aggregated or grouped based upon shared characteristics that may include inclusion in one or more additional predefined class or the presence of extracellular or intracellular markers, similar gene expression profile, nuclear antigens, enzymatic activity, protein expression and localization, cell cycle analysis, chromosomal analysis, cell volume, and morphological characteristics like granularity and size of nucleus or other distinguishing cellular characteristics.

In some embodiments, the physiological status of one or more cells is determined by examining and profiling the activation level of one or more activatable elements in a cellular pathway. In some embodiments, a cell is classified according to the activation level of a plurality of activatable elements. In some embodiments, a hematopoietic cell is classified according to the activation levels of a plurality of activatable elements. In some embodiments, 1, 2, 3, 4, 5, 6, 7, 8, 9, 10 or more activatable elements may be analyzed in a cell signaling pathway. In some embodiments, the activation levels of one or more activatable elements of a hematopoietic cell are correlated with a condition. In some embodiments, the activation levels of one or more activatable elements of a hematopoietic cell are correlated with a neoplastic or hematopoietic condition as described herein. Examples of hematopoietic cells include, but are not limited to, AML, MDS or MPDS cells.

In some embodiments, the activation level of one or more activatable elements in single cells in the sample is determined Cellular constituents that may include activatable elements include without limitation proteins, carbohydrates, lipids, nucleic acids and metabolites. The activatable element may be a portion of the cellular constituent, for example, an amino acid residue in a protein that may undergo phosphorylation, or it may be the cellular constituent itself, for example, a protein that is activated by translocation, change in conformation (due to, e.g., change in pH or ion concentration), by proteolytic cleavage, degradation through ubiquitination and the like. Upon activation, a change occurs to the activatable element, such as covalent modification of the activatable element (e.g., binding of a molecule or group to the activatable element, such as phosphorylation) or a conformational change. Such changes generally contribute to changes in particular biological, biochemical, or physical properties of the cellular constituent that contains the activatable element. The state of the cellular constituent that contains the activatable element is determined to some degree, though not necessarily completely, by the state of a particular activatable element of the cellular constituent. For example, a protein may have multiple activatable elements, and the particular activation states of these elements may overall determine the activation state of the protein; the state of a single activatable element is not necessarily determinative. Additional factors, such as the binding of other proteins, pH, ion concentration, interaction with other cellular constituents, and the like, can also affect the state of the cellular constituent.

In some embodiments, the activation levels of a plurality of intracellular activatable elements in single cells are determined. In some embodiments, at least about 2, 3, 4, 5, 6, 7, 8, 9, 10 or more than 10 intracellular activatable elements are determined

Activation states of activatable elements may result from chemical additions or modifications of biomolecules and include biochemical processes such as glycosylation, phosphorylation, acetylation, methylation, biotinylation, glutamylation, glycylation, hydroxylation, isomerization, prenylation, myristoylation, lipoylation, phosphopantetheinylation, sulfation, ISGylation, nitrosylation, palmitoylation, SUMOylation, ubiquitination, neddylation, citrullination, amidation, and disulfide bond formation, disulfide bond reduction. Other possible chemical additions or modifications of biomolecules include the formation of protein carbonyls, direct modifications of protein side chains, such as o-tyrosine, chloro-, nitrotyrosine, and dityrosine, and protein adducts derived from reactions with carbohydrate and lipid derivatives. Other modifications may be non-covalent, such as binding of a ligand or binding of an allosteric modulator.

One example of a covalent modification is the substitution of a phosphate group for a hydroxyl group in the side chain of an amino acid (phosphorylation). A wide variety of proteins are known that recognize specific protein substrates and catalyze the phosphorylation of serine, threonine, or tyrosine residues on their protein substrates. Such proteins are generally termed “kinases.” Substrate proteins that are capable of being phosphorylated are often referred to as phosphoproteins (after phosphorylation). Once phosphorylated, a substrate phosphoprotein may have its phosphorylated residue converted back to a hydroxyl one by the action of a protein phosphatase that specifically recognizes the substrate protein. Protein phosphatases catalyze the replacement of phosphate groups by hydroxyl groups on serine, threonine, or tyrosine residues. Through the action of kinases and phosphatases a protein may be reversibly phosphorylated on a multiplicity of residues and its activity may be regulated thereby. Thus, the presence or absence of one or more phosphate groups in an activatable protein is a preferred readout in the present invention.

Another example of a covalent modification of an activatable protein is the acetylation of histones. Through the activity of various acetylases and deacetylylases the DNA binding function of histone proteins is tightly regulated. Furthermore, histone acetylation and histone deactelyation have been linked with malignant progression. See Nature, 2004 May 27; 429(6990): 457-63.

Another form of activation involves cleavage of the activatable element. For example, one form of protein regulation involves proteolytic cleavage of a peptide bond. While random or misdirected proteolytic cleavage may be detrimental to the activity of a protein, many proteins are activated by the action of proteases that recognize and cleave specific peptide bonds. Many proteins derive from precursor proteins, or pro-proteins, which give rise to a mature isoform of the protein following proteolytic cleavage of specific peptide bonds. Many growth factors are synthesized and processed in this manner, with a mature isoform of the protein typically possessing a biological activity not exhibited by the precursor form. Many enzymes are also synthesized and processed in this manner, with a mature isoform of the protein typically being enzymatically active, and the precursor form of the protein being enzymatically inactive. This type of regulation is generally not reversible. Accordingly, to inhibit the activity of a proteolytically activated protein, mechanisms other than “reattachment” must be used. Inhibitors may also be used. Among the enzymes that are proteolytically activated are serine and cysteine proteases, including cathepsins and caspases respectively.

In one embodiment, the activatable enzyme is a cysteine aspartic acid specific protease (caspase). The caspases are an important class of proteases that mediate programmed cell death (referred to in the art as “apoptosis”). Caspases are constitutively present in most cells, residing in the cytosol as a single chain proenzyme. These are activated to fully functional proteases by a first proteolytic cleavage to divide the chain into large and small caspase subunits and a second cleavage to remove the N-terminal domain. The subunits assemble into a tetramer with two active sites (Green, Cell 94:695-698, 1998). Many other proteolytically activated enzymes, known in the art as “zymogens,” also find use in the instant invention as activatable elements.

In an alternative embodiment the activation of the activatable element involves prenylation of the element. By “prenylation”, and grammatical equivalents used herein, is meant the addition of any lipid group to the element. Common examples of prenylation include the addition of farnesyl groups, geranylgeranyl groups, myristoylation and palmitoylation. In general these groups are attached via thioether linkages to the activatable element, although other attachments may be used.

In alternative embodiment, activation of the activatable element is detected as intermolecular clustering of the activatable element. By “clustering” or “multimerization”, and grammatical equivalents used herein, is meant any reversible or irreversible association of one or more signal transduction elements. Clusters can be made up of 2, 3, 4, etc., elements. Clusters of two elements are termed dimers. Clusters of 3 or more elements are generally termed oligomers, with individual numbers of clusters having their own designation; for example, a cluster of 3 elements is a trimer, a cluster of 4 elements is a tetramer, etc.

Clusters can be made up of identical elements or different elements. Clusters of identical elements are termed “homo” dimers, while clusters of different elements are termed “hetero” clusters. Accordingly, a cluster can be a homodimer, as is the case for the β₂-adrenergic receptor.

Alternatively, a cluster can be a heterodimer, as is the case for GABA_{B-R}. In other embodiments, the cluster is a homotrimer, as in the case of TNFα, or a heterotrimer such the one formed by membrane-bound and soluble CD95 to modulate apoptosis. In further embodiments the cluster is a homo-oligomer, as in the case of Thyrotropin releasing hormone receptor, or a hetero-oligomer, as in the case of TGFβ1.

In a preferred embodiment, the activation or signaling potential of elements is mediated by clustering, irrespective of the actual mechanism by which the element's clustering is induced. For example, elements can be activated to cluster a) as membrane bound receptors by binding to ligands (ligands including both naturally occurring or synthetic ligands), b) as membrane bound receptors by binding to other surface molecules, or c) as intracellular (non-membrane bound) receptors binding to ligands.

In a preferred embodiment the activatable elements are membrane bound receptor elements that cluster upon ligand binding such as cell surface receptors. As used herein, “cell surface receptor” refers to molecules that occur on the surface of cells, interact with the extracellular environment, and transmit or transduce (through signals) the information regarding the environment intracellularly in a manner that may modulate cellular activity directly or indirectly, e.g., via intracellular second messenger activities or transcription of specific promoters, resulting in transcription of specific genes. One class of receptor elements includes membrane bound proteins, or complexes of proteins, which are activated to cluster upon ligand binding. As is known in the art, these receptor elements can have a variety of forms, but in general they comprise at least three domains. First, these receptors have a ligand-binding domain, which can be oriented either extracellularly or intracellularly, usually the former. Second, these receptors have a membrane-binding domain (usually a transmembrane domain), which can take the form of a seven pass transmembrane domain (discussed below in connection with G-protein-coupled receptors) or a lipid modification, such as myristylation, to one of the receptor's amino acids which allows for membrane association when the lipid inserts itself into the lipid bilayer. Finally, the receptor has an signaling domain, which is responsible for propagating the downstream effects of the receptor.

Examples of such receptor elements include hormone receptors, steroid receptors, cytokine receptors, such as IL1-α, IL-β, IL-2, IL-3, IL-4, IL-5, IL-6, IL-7, IL-8, IL-9, IL-10. IL-12, IL-15, IL-18, IL-21, CCR5, CCR7, CCR-1-10, CCL20, chemokine receptors, such as CXCR4, adhesion receptors and growth factor receptors, including, but not limited to, PDGF-R (platelet derived growth factor receptor), EGF-R (epidermal growth factor receptor), VEGF-R (vascular endothelial growth factor), uPAR (urokinase plasminogen activator receptor), ACHR (acetylcholine receptor), IgE-R (immunoglobulin E receptor), estrogen receptor, thyroid hormone receptor, integrin receptors (β1, β2, β3, β4, β5, β6, α1, α2, α3, α4, α5, α6), MAC-1 (β2 and cd11b), αVβ33, opioid receptors (mu and kappa), FC receptors, serotonin receptors (5-HT, 5-HT6,5-HT7), β-adrenergic receptors, insulin receptor, leptin receptor, TNF receptor (tissue-necrosis factor), statin receptors, FAS receptor, BAFF receptor, FLT3 LIGAND receptor, GMCSF receptor, and fibronectin receptor.

In a preferred embodiment the activatable element is a cytokine receptor. Cytokines are a family of soluble mediators of cell-to-cell communication that includes interleukins, interferons, and colony-stimulating factors. The characteristic features of cytokines lie in their pleiotropy and functional redundancy. Most of the cytokine receptors that constitute distinct superfamilies do not possess intrinsic protein tyrosine kinase domains, yet receptor stimulation usually invokes rapid tyrosine phosphorylation of intracellular proteins, including the receptors themselves. Many members of the cytokine receptor superfamily activate the Jak protein tyrosine kinase family, with resultant phosphorylation of the STAT family of transcription factors. IL-2, IL-4, IL-7 and Interferon γ have all been shown to activate Jak kinases (Frank et al. (1995) Proc. Natl. Acad. Sci. USA 92:7779-7783); Scharfe et al. (1995) Blood 86:2077-2085); (Bacon et al. (1995) Proc. Natl. Acad. Sci. USA 92:7307-7311); and (Sakatsume et al. (1995) J. Biol. Chem. 270:17528-17534). Events downstream of Jak phosphorylation have also been elucidated. For example, exposure of T lymphocytes to IL-2 has been shown to lead to the phosphorylation of signal transducers and activators of transcription (STAT) proteins STAT1α, STAT1β, and STAT3, as well as of two STAT-related proteins, p94 and p95. The STAT proteins translocate to the nucleus and bind to a specific DNA sequence, thus suggesting a mechanism by which IL-2 may activate specific genes involved in immune cell function (Frank et al. supra). Jak3 is associated with the gamma chain of the IL-2, IL-4, and IL-7 cytokine receptors (Fujii et al. (1995) Proc. Natl. Acad. Sci. 92:5482-5486) and (Musso et al. (1995) J. Exp. Med. 181:1425-1431). The Jak kinases have been shown to be activated by numerous ligands that signal via cytokine receptors such as, growth hormone, erythropoietin and IL-6 (Kishimoto (1994) Stem cells Suppl. 12:37-44). Preferred activatable elements are selected from the group p-STAT1, p-STAT3, p-STATS, p-STAT6, p-PLCγ2, p-S6, p-Akt, p-Erk, p-CREB, p-38, and NF-KBp-65.

In a preferred embodiment the activatable element is a member of tumor necrosis factor receptor superfamily, such as the Tumor necrosis factor alpha receptor. Tumor necrosis factor α (TNF-α or TNF-alpha) is a pleiotropic cytokine that is primarily produced by activated macrophages and lymphocytes but is also expressed in endothelial cells and other cell types. TNF-alpha is a major mediator of inflammatory, immunological, and pathophysiological reactions. (Grell, M., et al., (1995) Cell, 83:793-802). Two distinct forms of TNF exist, a 26 kDa membrane expressed form and the soluble 17 kDa cytokine which is derived from proteolytic cleavage of the 26 kDa form. The soluble TNF polypeptide is 157 amino acids long and is the primary biologically active molecule.

TNF-alpha exerts its biological effects through interaction with high-affinity cell surface receptors. Two distinct membrane TNF-alpha receptors have been cloned and characterized. These are a 55 kDa species, designated p55 TNF-R and a 75 kDa species designated p75 TNF-R (Corcoran. A. E., et al., (1994) Eur. J. Biochem., 223:831-840). The two TNF receptors exhibit 28% similarity at the amino acid level. This is confined to the extracellular domain and consists of four repeating cysteine-rich motifs, each of approximately 40 amino acids. Each motif contains four to six cysteines in conserved positions. Dayhoff analysis shows the greatest intersubunit similarity among the first three repeats in each receptor. This characteristic structure is shared with a number of other receptors and cell surface molecules, which comprise the TNF-R/nerve growth factor receptor superfamily (Corcoran. A. E., et al., (1994) Eur. J. Biochem., 223:831-840).

TNF signaling is initiated by receptor clustering, either by the trivalent ligand TNF or by cross-linking monoclonal antibodies (Vandevoorde, V., et al., (1997) J. Cell Biol., 137:1627-1638). Crystallographic studies of TNF and the structurally related cytokine, lymphotoxin (LT), have shown that both cytokines exist as homotrimers, with subunits packed edge to edge in threefold symmetry. Structurally, neither TNF or LT reflect the repeating pattern of the their receptors. Each monomer is cone shaped and contains two hydrophilic loops on opposite sides of the base of the cone. Recent crystal structure determination of a p55 soluble TNF-R/LT complex has confirmed the hypothesis that loops from adjacent monomers join together to form a groove between monomers and that TNF-R binds in these grooves (Corcoran. A. E., et al., (1994) Eur. J. Biochem., 223:831-840).

In one embodiment, the activatable element is a receptor tyrosine kinase (RTK). The approximately 60 RTKs identified can be divided into 20 subfamilies defined by a prototypical receptor and/or ligand (Pawson European Journal of Cancer Vol. 38 Suppl. 5 (2002) S3-510). Receptor tyrosine kinases subgroups have structural similarities in their extracellular domains as well as in the organization of the tyrosine kinase catalytic region within their cytoplasmic domains. Sub-group I (epidermal growth factor (EGF) receptor and related receptors), sub-group II (insulin receptor and related receptors) sub-group VI (Trk family members) and sub-group XIII (Ephrin receptor family members) contain cysteine-rich sequences (Hirai et al., (1987) Science 238:1717-1720 and Lindberg and Hunter, (1990) Mol. Cell. Biol. 10:6316-6324, Pawson European Journal of Cancer Vol. 38 Suppl. 5 (2002) S3S10). Subgroups III (platelet-derived growth factor (PDGF) receptor and related receptors) and IV (the fibroblast growth factor (FGF) receptors) are characterized as having immunoglobulin (Ig)-like folds in their extracellular domains, as well as having their kinase domains split by an inter-kinase domain which is a variable stretch of unrelated amino acids (Yanden and Ullrich (1988) supra and Hanks et al. (1988) supra). All other RTK sub-groups have a cytoplasmic domain in which the kinase domain is encoded as a contiguous sequence (Hanks et al. (1988) Science 241:42-52).

In another embodiment the receptor element is a member of the hematopoietin receptor superfamily. Hematopoietin receptor superfamily is used herein to define single-pass transmembrane receptors, with a three-domain architecture: an extracellular domain that binds the activating ligand, a short transmembrane segment, and a domain residing in the cytoplasm. The extracellular domains of these receptors have low but significant homology within their extracellular ligand-binding domain comprising about 200-210 amino acids. The homologous region is characterized by four cysteine residues located in the N-terminal half of the region, and a Trp-Ser-X-Trp-Ser (WSXWS) motif located just outside the membrane-spanning domain. Further structural and functional details of these receptors are provided by Cosman, D. et al., (1990). The receptors of IL-1, IL-2, IL-3, IL-4, IL-5, IL-6, IL-7, prolactin, placental lactogen, growth hormone GM-CSF, G-CSF, M-CSF and erythropoietin have, for example, been identified as members of this receptor family.

In a further embodiment, the receptor element is an integrin other than Leukocyte Function Antigen-1 (LFA-1). Members of the integrin family of receptors function as heterodimers, composed of various α and β subunits, and mediate interactions between a cell's cytoskeleton and the extracellular matrix. (Reviewed in, Giancotti and Ruoslahti, Science 285, 13 Aug. 1999). Different combinations of the α and β subunits give rise to a wide range of ligand specificities, which may be increased further by the presence of cell-type-specific factors. Integrin clustering is known to activate a number of intracellular signals, such as RAS, MAP kinase, and phosphotidylinosital-3-kinase. In a preferred embodiment the receptor element is a heterodimer (other than LFA-1) composed of a β integrin and an α integrin chosen from the following integrins; β1, β2, β3, β4, β5, β6, α1, α2, α3, α4, α5, and α6, or is MAC-1 (β2 and cd11b), or αVβ3.

In a preferred embodiment the element is an intracellular adhesion molecule (ICAM). ICAMs-1, -2, and -3 are cellular adhesion molecules belonging to the immunogloblin superfamily. Each of these receptors has a single membrane-spanning domain and all bind to β2 integrins via extracellular binding domains similar in structure to Ig-loops. (Signal Transduction, Gomperts, et al., eds, Academic Press Publishers, 2002, Chapter 14, pp 318-319).

In another embodiment the activatable elements cluster for signaling by contact with other surface molecules. In contrast to the receptors discussed above, these elements cluster for signaling by contact with other surface molecules, and generally use molecules presented on the surface of a second cell as ligands. Receptors of this class are important in cell-cell interactions, such mediating cell-to-cell adhesion and immunorecognition.

Examples of such receptor elements are CD3 (T cell receptor complex), BCR (B cell receptor complex), CD4, CD28, CD80, CD86, CD54, CD102, CD50 and ICAMs 1, 2 and 3.

In a preferred embodiment the receptor element is a T cell receptor complex (TCR). TCRs occur as either of two distinct heterodimers, αβ, or γξ both of which are expressed with the non-polymorphic CD3 polypeptides γ, δ, ε, and the ξ chain. The CD3 polypeptides, and ξ chain, are critical for intracellular signaling. The αβ TCR heterodimer expressing cells predominate in most lymphoid compartments and are responsible for the classical helper or cytotoxic T cell responses. In most cases, the αβ TCR ligand is a peptide antigen bound to a class I or a class II MHC molecule (Fundamental Immunology, fourth edition, W. E. Paul, ed., Lippincott-Raven Publishers, 1999, Chapter 10, pp 341-367).

In another embodiment, the activatable element is a member of the large family of G-protein-coupled receptors. It has recently been reported that a G-protein-coupled receptors are capable of clustering. (Kroeger, et al., J Biol Chem 276:16, 12736-12743, Apr. 20, 2001; Bai, et al., J Biol Chem 273:36, 23605-23610, Sep. 4, 1998; Rocheville, et al., J Biol Chem 275 (11), 7862-7869, Mar. 17, 2000). As used herein G-protein-coupled receptor, and grammatical equivalents thereof, refers to the family of receptors that bind to heterotrimeric “G proteins.” Many different G proteins are known to interact with receptors. G protein signaling systems include three components: the receptor itself, a GTP-binding protein (G protein), and an intracellular target protein. The cell membrane acts as a switchboard. Messages arriving through different receptors can produce a single effect if the receptors act on the same type of G protein. On the other hand, signals activating a single receptor can produce more than one effect if the receptor acts on different kinds of G proteins, or if the G proteins can act on different effectors.

In their resting state, the G proteins, which consist of alpha (α), beta (β) and gamma (γ) subunits, are complexed with the nucleotide guanosine diphosphate (GDP) and are in contact with receptors. When a hormone or other first messenger binds to a receptor, the receptor changes conformation and this alters its interaction with the G protein. This spurs a subunit to release GDP, and the more abundant nucleotide guanosine triphosphate (GTP), replaces it, activating the G protein. The G protein then dissociates to separate the α subunit from the still complexed beta and gamma subunits. Either the Gα subunit, or the Gβγ complex, depending on the pathway, interacts with an effector. The effector (which is often an enzyme) in turn converts an inactive precursor molecule into an active “second messenger,” which may diffuse through the cytoplasm, triggering a metabolic cascade. After a few seconds, the Gα converts the GTP to GDP, thereby inactivating itself. The inactivated Gα may then reassociate with the Gβγ complex.

Hundreds, if not thousands, of receptors convey messages through heterotrimeric G proteins, of which at least 17 distinct forms have been isolated. Although the greatest variability has been seen in a subunit, several different β and γ structures have been reported. There are, additionally, many different G protein-dependent effectors.

Most G protein-coupled receptors are comprised of a single protein chain that passes through the plasma membrane seven times. Such receptors are often referred to as seven-transmembrane receptors (STRs). More than a hundred different STRs have been found, including many distinct receptors that bind the same ligand, and there are likely many more STRs awaiting discovery.

In addition, STRs have been identified for which the natural ligands are unknown; these receptors are termed “orphan” G protein-coupled receptors, as described above. Examples include receptors cloned by Neote et al. (1993) Cell 72, 415; Kouba et al. FEBS Lett. (1993)321, 173; and Birkenbach et al. (1993) J. Virol. 67, 2209.

Known ligands for G protein coupled receptors include: purines and nucleotides, such as adenosine, cAMP, ATP, UTP, ADP, melatonin and the like; biogenic amines (and related natural ligands), such as 5-hydroxytryptamine, acetylcholine, dopamine, adrenaline, histamine, noradrenaline, tyramine/octopamine and other related compounds; peptides such as adrenocorticotrophic hormone (acth), melanocyte stimulating hormone (msh), melanocortins, neurotensin (nt), bombesin and related peptides, endothelins, cholecystokinin, gastrin, neurokinin b (nk3), invertebrate tachykinin-like peptides, substance k (nk2), substance p (nk1), neuropeptide y (npy), thyrotropin releasing-factor (trf), bradykinin, angiotensin ii, beta-endorphin, c5a anaphalatoxin, calcitonin, chemokines (also called intercrines), corticotrophic releasing factor (crf), dynorphin, endorphin, fmlp and other formylated peptides, follitropin (fsh), fungal mating pheromones, galanin, gastric inhibitory polypeptide receptor (gip), glucagon-like peptides (glps), glucagon, gonadotropin releasing hormone (gnrh), growth hormone releasing hormone (ghrh), insect diuretic hormone, interleukin-8, leutropin (1 h/hcg), met-enkephalin, opioid peptides, oxytocin, parathyroid hormone (pth) and pthrp, pituitary adenylyl cyclase activating peptide (pacap), secretin, somatostatin, thrombin, thyrotropin (tsh), vasoactive intestinal peptide (vip), vasopressin, vasotocin; eicosanoids such as ip-prostacyclin, pg-prostaglandins, tx-thromboxanes; retinal based compounds such as vertebrate 11-cis retinal, invertebrate 11-cis retinal and other related compounds; lipids and lipid-based compounds such as cannabinoids, anandamide, lysophosphatidic acid, platelet activating factor, leukotrienes and the like; excitatory amino acids and ions such as calcium ions and glutamate.

Preferred G protein coupled receptors include, but are not limited to: α1-adrenergic receptor, α1β-adrenergic receptor, α2-adrenergic receptor, α2B-adrenergic receptor, β1-adrenergic receptor, β2-adrenergic receptor, β3-adrenergic receptor, m1 acetylcholine receptor (AChR), m2 AChR, m3 AChR, m4 AChR, m5 AChR, D1 dopamine receptor, D2 dopamine receptor, D3 dopamine receptor, D4 dopamine receptor, D5 dopamine receptor, A1 adenosine receptor, A2a adenosine receptor, A2b adenosine receptor, A3 adenosine receptor, 5-HT1a receptor, 5-HT1b receptor, 5HT1-like receptor, 5-HT1d receptor, 5HT1d-like receptor, 5HT1d beta receptor, substance K (neurokinin A) receptor, fMLP receptor (FPR), fMLP-like receptor (FPRL-1), angiotensin II type 1 receptor, endothelin ETA receptor, endothelin ETB receptor, thrombin receptor, growth hormone-releasing hormone (GHRH) receptor, vasoactive intestinal peptide receptor, oxytocin receptor, somatostatin SSTR1 and SSTR2, SSTR3, cannabinoid receptor, follicle stimulating hormone (FSH) receptor, leutropin (LH/HCG) receptor, thyroid stimulating hormone (TSH) receptor, thromboxane A2 receptor, platelet-activating factor (PAF) receptor, C5a anaphylatoxin receptor, CXCR1 (IL-8 receptor A), CXCR2 (IL-8 receptor B), Delta Opioid receptor, Kappa Opioid receptor, mip-1alpha/RANTES receptor (CRR1), Rhodopsin, Red opsin, Green opsin, Blue opsin, metabotropic glutamate mGluR1-6, histamine H2 receptor, ATP receptor, neuropeptide Y receptor, amyloid protein precursor receptor, insulin-like growth factor II receptor, bradykinin receptor, gonadotropin-releasing hormone receptor, cholecystokinin receptor, melanocyte stimulating hormone receptor, antidiuretic hormone receptor, glucagon receptor, and adrenocorticotropic hormone II receptor. In addition, there are at least five receptors (CC and CXC receptors) involved in HIV viral attachment to cells. The two major co-receptors for HIV are CXCR4, (fusin receptor, LESTR, SDF-1α receptor) and CCR5 (m-trophic). More preferred receptors include the following human receptors: melatonin receptor 1a, galanin receptor 1, neurotensin receptor, adenosine receptor 2a, somatostatin receptor 2 and corticotropin releasing factor receptor 1. Melatonin receptor 1a is particularly preferred. Other G protein coupled receptors (GPCRs) are known in the art.

In one embodiment, Lnk is a protein to be measured. Hematopoietic stem cells (HSCs) give rise to variety of hematopoietic cells via pluripotential progenitors. Lineage-committed progenitors are responsible for blood production throughout adult life. Amplification of HSCs or progenitors represents a potentially powerful approach to the treatment of various blood disorders Animal model studies demonstrated that Lnk acts as a broad inhibitor of signaling pathways in hematopoietic lineages. Lnk is an adaptor protein which belongs to a family of proteins sharing several structural motifs, including a Src homology 2 (SH2) domain which binds phospho-tyrosines in various signal-transducing proteins. The SH2 domain is essential for Lnk-mediated negative regulation of several cytokine receptors (i.e. Mp1, EpoR, c-Kit, Il-3R and IL7R). Therefore, inhibition of the binding of Lnk to cytokine receptors might lead to enhanced downstream signaling of the receptor and thereby to improved hematopoiesis in response to exposure to cytokines (i.e. erythropoietin in anemic patients). (Gueller et al, Adaptor protein Lnk associates with Y568 in c-Kit. 1: Biochem J. 2008 Jun. 30.) It has been shown that overexpression of Lnk in Ba/F3-MPLW515L cells inhibits cytokine-independent growth, while suppression of Lnk in UT7-MPLW515L cells enhances proliferation. Lnk blocks the activation of Jak2, Stat3, Erk, and Akt in these cells. (Gery et al., Adaptor protein Lnk negatively regulates the mutant MPL, MPLW515L associated with myeloproliferative disorders, Blood, 1 Nov. 2007, Vol. 110, No. 9, pp. 3360-3364.) Thus, Lnk is an important protein to measure for the evaluation of AML/MDS/MPS.

In one embodiment, the activatable elements are intracellular receptors capable of clustering. Elements of this class are not membrane-bound. Instead, they are free to diffuse through the intracellular matrix where they bind soluble ligands prior to clustering and signal transduction. In contrast to the previously described elements, many members of this class are capable of binding DNA after clustering to directly effect changes in RNA transcription.

In another embodiment the intracellular receptors capable of clustering are perioxisome proliferator-activated receptors (PPAR). PPARs are soluble receptors responsive to lipophillic compounds, and induce various genes involved in fatty acid metabolism. The three PPAR subtypes, PPAR α, β, and γ have been shown to bind to DNA after ligand binding and heterodimerization with retinoid X receptor. (Summanasekera, et al., J Biol Chem, M211261200, Dec. 13, 2002.)

In another embodiment the activatable element is a nucleic acid. Activation and deactivation of nucleic acids can occur in numerous ways including, but not limited to, cleavage of an inactivating leader sequence as well as covalent or non-covalent modifications that induce structural or functional changes. For example, many catalytic RNAs, e.g. hammerhead ribozymes, can be designed to have an inactivating leader sequence that deactivates the catalitic activity of the ribozyme until cleavage occurs. An example of a covalent modification is methylation of DNA. Deactivation by methylation has been shown to be a factor in the silencing of certain genes, e.g. STAT regulating SOCS genes in lymphomas. See Leukemia. See February 2004; 18(2): 356-8. SOCS1 and SHP1 hypermethylation in mantle cell lymphoma and follicular lymphoma: implications for epigenetic activation of the Jak/STAT pathway. Chim C S, Wong K Y, Loong F, Srivastava G.

In another embodiment the activatable element is a small molecule, carbohydrate, lipid or other naturally occurring or synthetic compound capable of having an activated isoform. In addition, as pointed out above, activation of these elements need not include switching from one form to another, but can be detected as the presence or absence of the compound. For example, activation of cAMP (cyclic adenosine mono-phosphate) can be detected as the presence of cAMP rather than the conversion from non-cyclic AMP to cyclic AMP.

Examples of proteins that may include activatable elements include, but are not limited to kinases, phosphatases, lipid signaling molecules, adaptor/scaffold proteins, cytokines, cytokine regulators, ubiquitination enzymes, adhesion molecules, cytoskeletal/contractile proteins, heterotrimeric G proteins, small molecular weight GTPases, guanine nucleotide exchange factors, GTPase activating proteins, caspases, proteins involved in apoptosis, cell cycle regulators, molecular chaperones, metabolic enzymes, vesicular transport proteins, hydroxylases, isomerases, deacetylases, methylases, demethylases, tumor suppressor genes, proteases, ion channels, molecular transporters, transcription factors/DNA binding factors, regulators of transcription, and regulators of translation. Examples of activatable elements, activation states and methods of determining the activation level of activatable elements are described in US Publication Number 20060073474 entitled “Methods and compositions for detecting the activation state of multiple proteins in single cells” and US Publication Number 20050112700 entitled “Methods and compositions for risk stratification” the content of which are incorporate here by reference. See also U.S. Ser. Nos. 61/048,886; 61/048,920; and Shulz et al., Current Protocols in Immunology 2007, 78:8.17.1-20.

In some embodiments, the protein is selected from the group consisting of HER receptors, PDGF receptors, Kit receptor, FGF receptors, Eph receptors, Trk receptors, IGF receptors, Insulin receptor, Met receptor, Ret, VEGF receptors, TIE1, TIE2, FAK, Jak1, Jak2, Jak3, Tyk2, Src, Lyn, Fyn, Lck, Fgr, Yes, Csk, Ab1, Btk, ZAP70, Syk, IRAKs, cRaf, ARaf, BRAF, Mos, Lim kinase, ILK, Tp1, ALK, TGFβ receptors, BMP receptors, MEKKs, ASK, MLKs, DLK, PAKs, Mek 1, Mek 2, MKK3/6, MKK4/7, ASK1,Cot, NIK, Bub, Myt 1, Wee1, Casein kinases, PDK1, SGK1, SGK2, SGK3, Akt1, Akt2, Akt3, p90Rsks, p70S6 Kinase, Prks, PKCs, PKAs, ROCK 1, ROCK 2, Auroras, CaMKs, MNKs, AMPKs, MELK, MARKs, Chk1, Chk2, LKB-1, MAPKAPKs, Pim1, Pim2, Pim3, IKKs, Cdks, Jnks, Erks, IKKs, GSK3α, GSK3β, Cdks, CLKs, PKR, PI3-Kinase class 1, class 2, class 3, mTor, SAPK/JNK1,2,3, p38s, PKR, DNA-PK, ATM, ATR, Receptor protein tyrosine phosphatases (RPTPs), LAR phosphatase, CD45, Non receptor tyrosine phosphatases (NPRTPs), SHPs, MAP kinase phosphatases (MKPs), Dual Specificity phosphatases (DUSPs), CDC25 phosphatases, Low molecular weight tyrosine phosphatase, Eyes absent (EYA) tyrosine phosphatases, Slingshot phosphatases (SSH), serine phosphatases, PP2A, PP2B, PP2C, PP1, PP5, inositol phosphatases, PTEN, SHIPs, myotubularins, phosphoinositide kinases, phopsholipases, prostaglandin synthases, 5-lipoxygenase, sphingosine kinases, sphingomyelinases, adaptor/scaffold proteins, Shc, Grb2, BLNK, LAT, B cell adaptor for PI3-kinase (BCAP), SLAP, Dok, KSR, MyD88, Crk, CrkL, GAD, Nck, Grb2 associated binder (GAB), Fas associated death domain (FADD), TRADD, TRAF2, RIP, T-Cell leukemia family, IL-2, IL-4, IL-8, IL-6, interferon β, interferon α, suppressors of cytokine signaling (SOCs), Cb1, SCF ubiquitination ligase complex, APC/C, adhesion molecules, integrins, Immunoglobulin-like adhesion molecules, selectins, cadherins, catenins, focal adhesion kinase, p130CAS, fodrin, actin, paxillin, myosin, myosin binding proteins, tubulin, eg5/KSP, CENPs, β-adrenergic receptors, muscarinic receptors, adenylyl cyclase receptors, small molecular weight GTPases, H-Ras, K-Ras, N-Ras, Ran, Rac, Rho, Cdc42, Arfs, RABs, RHEB, Vav, Tiam, Sos, Db1, PRK, TSC1,2, Ras-GAP, Arf-GAPs, Rho-GAPs, caspases, Caspase 2, Caspase 3, Caspase 6, Caspase 7, Caspase 8, Caspase 9, Bcl-2, Mcl-1, Bcl-XL, Bcl-w, Bcl-B, A1, Bax, Bak, Bok, Bik, Bad, Bid, Bim, Bmf, Hrk, Noxa, Puma, IAPs, XIAP, Smac, Cdk4, Cdk 6, Cdk 2, Cdk1, Cdk 7, Cyclin D, Cyclin E, Cyclin A, Cyclin B, Rb, p16, p14Arf, p27KIP, p21CIP, molecular chaperones, Hsp90s, Hsp70, Hsp27, metabolic enzymes, Acetyl-CoAa Carboxylase, ATP citrate lyase, nitric oxide synthase, caveolins, endosomal sorting complex required for transport (ESCRT) proteins, vesicular protein sorting (Vsps), hydroxylases, prolyl-hydroxylases PHD-1, 2 and 3, asparagine hydroxylase FIH transferases, Pin1 prolyl isomerase, topoisomerases, deacetylases, Histone deacetylases, sirtuins, histone acetylases, CBP/P300 family, MYST family, ATF2, DNA methyl transferases, Histone H3K4 demethylases, H3K27, JHDM2A, UTX, VHL, WT-1, p53, Hdm, PTEN, ubiquitin proteases, urokinase-type plasminogen activator (uPA) and uPA receptor (uPAR) system, cathepsins, metalloproteinases, esterases, hydrolases, separase, potassium channels, sodium channels, multi-drug resistance proteins, P-Glycoprotein, nucleoside transporters, Ets, Elk, SMADs, Rel-A (p65-NFKB), CREB, NFAT, ATF-2, AFT, Myc, Fos, Sp1, Egr-1, T-bet, β-catenin, HIFs, FOXOs, E2Fs, SRFs, TCFs, Egr-1, \tilde{β}-catenin, FOXO STAT1, STAT 3, STAT 4, STAT 5, STAT 6, p53, WT-1, HMGA, pS6, 4EPB-1, eIF4E-binding protein, RNA polymerase, initiation factors, elongation factors.

Generally, the methods of the invention involve determining the activation levels of an activatable element in a plurality of single cells in a sample. The activation levels can be obtained by perturbing the cell state using a modulator.

### Signaling Pathways

In some embodiments, the methods of the invention are employed to determine the status of an activatable element in a signaling pathway. In some embodiments, a cell is classified, as described herein, according to the activation level of one or more activatable elements in one or more signaling pathways. Signaling pathways and their members have been described. See (Hunter T. Cell Jan. 7, 2000; 100(1): 13-27). Exemplary signaling pathways include the following pathways and their members: The MAP kinase pathway including Ras, Raf, MEK, ERK and elk; the PI3K/Akt pathway including PI-3-kinase, PDK1, Akt and Bad; the NF-κB pathway including IKKs, IkB and the Wnt pathway including frizzled receptors, beta-catenin, APC and other co-factors and TCF (see Cell Signaling Technology, Inc. 2002 Catalog pages 231-279 and Hunter T., supra.). In some embodiments of the invention, the correlated activatable elements being assayed (or the signaling proteins being examined) are members of the MAP kinase, Akt, NFkB, WNT, RAS/RAF/MEK/ERK, JNK/SAPK, p38 MAPK, Src Family Kinases, JAK/STAT and/or PKC signaling pathways.

In some embodiments, the methods of the invention are employed to determine the status of a signaling protein in a signaling pathway known in the art including those described herein. Exemplary types of signaling proteins within the scope of the present invention include, but are not limited to kinases, kinase substrates (i.e. phosphorylated substrates), phosphatases, phosphatase substrates, binding proteins (such as 14-3-3), receptor ligands and receptors (cell surface receptor tyrosine kinases and nuclear receptors)). Kinases and protein binding domains, for example, have been well described (see, e.g., Cell Signaling Technology, Inc., 2002 Catalogue “The Human Protein Kinases” and “Protein Interaction Domains” pgs. 254-279).

Nuclear Factor-kappaB (NF-κB) Pathway: Nuclear factor-kappaB (NF-kappaB) transcription factors and the signaling pathways that activate them are central coordinators of innate and adaptive immune responses. More recently, it has become clear that NF-kappaB signaling also has a critical role in cancer development and progression. NF-kappaB provides a mechanistic link between inflammation and cancer, and is a major factor controlling the ability of both pre-neoplastic and malignant cells to resist apoptosis-based tumor-surveillance mechanisms. In mammalian cells, there are five NF-κB family members, RelA (p65), RelB, c-Rel, p50/p105 (NF-κB 1) and p52/p 100 (NF-κB2) and different NF-κB complexes are formed from their homo and heterodimers. In most cell types, NF-κB complexes are retained in the cytoplasm by a family of inhibitory proteins known as inhibitors of NF-κB (IκBs). Activation of NF-κB typically involves the phosphorylation of IκB by the IκB kinase (IKK) complex, which results in IκB ubiquitination with subsequent degradation. This releases NF-κB and allows it to translocate freely to the nucleus. The genes regulated by NF-κB include those controlling programmed cell death, cell adhesion, proliferation, the innate- and adaptive-immune responses, inflammation, the cellular-stress response and tissue remodeling. However, the expression of these genes is tightly coordinated with the activity of many other signaling and transcription-factor pathways. Therefore, the outcome of NF-κB activation depends on the nature and the cellular context of its induction. For example, it has become apparent that NF-κB activity can be regulated by both oncogenes and tumor suppressors, resulting in either stimulation or inhibition of apoptosis and proliferation. See Perkins, N. Integrating cell-signaling pathways with NF-κB and IKK function. Reviews: Molecular Cell Biology. January, 2007; 8(1): 49-62, hereby fully incorporated by reference in its entirety for all purposes. Hayden, M. Signaling to NF-κB. Genes & Development. 2004; 18: 2195-2224, hereby fully incorporated by reference in its entirety for all purposes. Perkins, N. Good Cop, Bad Cop: The Different Faces of NF-κB. Cell Death and Differentiation. 2006; 13: 759-772, hereby fully incorporated by reference in its entirety for all purposes.

Phosphatidylinositol 3-kinase (PI3-K)/AKT Pathway: PI3-Ks are activated by a wide range of cell surface receptors to generate the lipid second messengers phosphatidylinositol 3,4-biphosphate (PIP₂) and phosphatidylinositol 3,4,5-trisphosphate (PIP_{S}). Examples of receptor tyrosine kinases include but are not limited to FLT3 LIGAND, EGFR, IGF-1R, HER2/neu, VEGFR, and PDGFR. The lipid second messengers generated by PI3Ks regulate a diverse array of cellular functions. The specific binding of PI3,4P₂ and PI3,4,5P₃ to target proteins is mediated through the pleckstrin homology (PH) domain present in these target proteins. One key downstream effector of PI3-K is Akt, a serine/threonine kinase, which is activated when its PH domain interacts with PI3,4P₂ and PI3,4,5P₃ resulting in recruitment of Akt to the plasma membrane. Once there, in order to be fully activated, Akt is phosphorylated at threonine 308 by 3-phosphoinositide-dependent protein kinase-1 (PDK-1) and at serine 473 by several PDK2 kinases. Akt then acts downstream of PI3K to regulate the phosphorylation of a number of substrates, including but not limited to forkhead box O transcription factors, Bad, GSK-3β, I-κB, mTOR, MDM-2, and S6 ribosomal subunit. These phosphorylation events in turn mediate cell survival, cell proliferation, membrane trafficking, glucose homeostasis, metabolism and cell motility. Deregulation of the PI3K pathway occurs by activating mutations in growth factor receptors, activating mutations in a PI3-K gene (e.g. PIK3CA), loss of function mutations in a lipid phosphatase (e.g. PTEN), up-regulation of Akt, or the impairment of the tuberous sclerosis complex (TSC1/2). All these events are linked to increased survival and proliferation. See Vivanco, I. The Phosphatidylinositol 3-Kinase-AKT Pathway in Human Cancer. Nature Reviews: Cancer. July, 2002; 2: 489-501 and Shaw, R. Ras, PI(3)K and mTOR signaling controls tumor cell growth. Nature. May, 2006; 441: 424-430, Marone et al., Biochimica et Biophysica Acta, 2008; 1784, p159-185 hereby fully incorporated by reference in their entirety for all purposes.

Wnt Pathway: The Wnt signaling pathway describes a complex network of proteins well known for their roles in embryogenesis, normal physiological processes in adult animals, such as tissue homeostasis, and cancer. Further, a role for the Wnt pathway has been shown in self-renewal of hematopoietic stem cells (Reya T et al., Nature. 2003 May 22; 423(6938):409-14). Cytoplasmic levels of β-catenin are normally kept low through the continuous proteosomal degradation of β-catenin controlled by a complex of glycogen synthase kinase 3β (GSK-3β, axin, and adenomatous polyposis coli (APC). When Wnt proteins bind to a receptor complex composed of the Frizzled receptors (Fz) and low density lipoprotein receptor-related protein (LRP) at the cell surface, the GSK-3/axin/APC complex is inhibited. Key intermediates in this process include disheveled (Dsh) and axin binding the cytoplasmic tail of LRP. Upon Wnt signaling and inhibition of the β-catenin degradation pathway, β-catenin accumulates in the cytoplasm and nucleus. Nuclear β-catenin interacts with transcription factors such as lymphoid enhanced-binding factor 1 (LEF) and T cell-specific transcription factor (TCF) to affect transcription of target genes. See Gordon, M. Wnt Signaling: Multiple Pathways, Multiple Receptors, and Multiple Transcription Factors. J of Biological Chemistry. June, 2006; 281(32): 22429-22433, Logan C Y, Nusse R: The Wnt signaling pathway in development and disease. Annu Rev Cell Dev Biol 2004, 20:781-810, Clevers H: Wnt/beta-catenin signaling in development and disease. Cell 2006, 127:469-480. hereby fully incorporated by reference in its entirety for all purposes.

Protein Kinase C (PKC) Signaling: The PKC family of serine/threonine kinases mediate signaling pathways following activation of receptor tyrosine kinases, G-protein coupled receptors and cytoplasmic tyrosine kinases. Activation of PKC family members is associated with cell proliferation, differentiation, survival, immune function, invasion, migration and angiogenesis. Disruption of PKC signaling has been implicated in tumorigenesis and drug resistance. PKC isoforms have distinct and overlapping roles in cellular functions. PKC was originally identified as a phospholipid and calcium-dependent protein kinase. The mammalian PKC superfamily consists of 13 different isoforms that are divided into four subgroups on the basis of their structural differences and related cofactor requirements cPKC (classical PKC) isoforms (α, βI, βII and γ), which respond both to Ca2+ and DAG (diacylglycerol), nPKC (novel PKC) isoforms (δ, ε, θ and η), which are insensitive to Ca2+, but dependent on DAG, atypical PKCs (aPKCs, ↓/λ, ζ), which are responsive to neither co-factor, but may be activated by other lipids and through protein-protein interactions, and the related PKN (protein kinase N) family (e.g. PKN1, PKN2 and PKN3), members of which are subject to regulation by small GTPases. Consistent with their different biological functions, PKC isoforms differ in their structure, tissue distribution, subcellular localization, mode of activation and substrate specificity. Before maximal activation of its kinase, PKC requires a priming phosphorylation which is provided constitutively by phosphoinositide-dependent kinase 1 (PDK-1). The phospholipid DAG has a central role in the activation of PKC by causing an increase in the affinity of classical PKCs for cell membranes accompanied by PKC activation and the release of an inhibitory substrate (a pseudo-substrate) to which the inactive enzyme binds. Activated PKC then phosphorylates and activates a range of kinases. The downstream events following PKC activation are poorly understood, although the MEK-ERK (mitogen activated protein kinase kinase-extracellular signal-regulated kinase) pathway is thought to have an important role. There is also evidence to support the involvement of PKC in the PI3K-Akt pathway. PKC isoforms probably form part of the multi-protein complexes that facilitate cellular signal transduction. Many reports describe dysregulation of several family members. For example alterations in PKCε have been detected in thyroid cancer, and have been correlated with aggressive, metastatic breast cancer and PKCι was shown to be associated with poor outcome in ovarian cancer. (Knauf J A, et al. Isozyme-Specific Abnormalities of PKC in Thyroid Cancer Evidence for Post-Transcriptional Changes in PKC Epsilon. The Journal of Clinical Endocrinology & Metabolism. Vol. 87, No. 5, pp 2150-2159; Zhang L et al. Integrative Genomic Analysis of Protein Kinase C (PKC) Family Identifies PKC{iota} as a Biomarker and Potential Oncogene in Ovarian Carcinoma. Cancer Res. 2006, Vol 66, No. 9, pp 4627±4635)

Mitogen Activated Protein (MAP) Kinase Pathways: MAP kinases transduce signals that are involved in a multitude of cellular pathways and functions in response to a variety of ligands and cell stimuli. (Lawrence et al., Cell Research (2008) 18: 436-442). Signaling by MAPKs affects specific events such as the activity or localization of individual proteins, transcription of genes, and increased cell cycle entry, and promotes changes that orchestrate complex processes such as embryogenesis and differentiation. Aberrant or inappropriate functions of MAPKs have now been identified in diseases ranging from cancer to inflammatory disease to obesity and diabetes. MAPKs are activated by protein kinase cascades consisting of three or more protein kinases in series: MAPK kinase kinases (MAP3Ks) activate MAPK kinases (MAP2Ks) by dual phosphorylation on S/T residues; MAP2Ks then activate MAPKs by dual phosphorylation on Y and T residues MAPKs then phosphorylate target substrates on select S/T residues typically followed by a proline residue. In the ERK1/2 cascade the MAP3K is usually a member of the Raf family. Many diverse MAP3Ks reside upstream of the p38 and the c-Jun N-terminal kinase/stress-activated protein kinase (JNK/SAPK) MAPK groups, which have generally been associated with responses to cellular stress. Downstream of the activating stimuli, the kinase cascades may themselves be stimulated by combinations of small G proteins, MAP4Ks, scaffolds, or oligomerization of the MAP3K in a pathway. In the ERK1/2 pathway, Ras family members usually bind to Raf proteins leading to their activation as well as to the subsequent activation of other downstream members of the pathway.

Ras/RAF/MEK/ERK Pathway: Classic activation of the RAS/Raf/MAPK cascade occurs following ligand binding to a receptor tyrosine kinase at the cell surface, but a vast array of other receptors have the ability to activate the cascade as well, such as integrins, serpentine receptors, heterotrimeric G-proteins, and cytokine receptors. Although conceptually linear, considerable cross talk occurs between the Ras/Raf/MAPK/Erk kinase (MEK)/Erk MAPK pathway and other MAPK pathways as well as many other signaling cascades. The pivotal role of the Ras/Raf/MEK/Erk MAPK pathway in multiple cellular functions underlies the importance of the cascade in oncogenesis and growth of transformed cells. As such, the MAPK pathway has been a focus of intense investigation for therapeutic targeting. Many receptor tyrosine kinases are capable of initiating MAPK signaling. They do so after activating phosphorylation events within their cytoplasmic domains provide docking sites for src-homology 2 (SH2) domain-containing signaling molecules. Of these, adaptor proteins such as Grb2 recruit guanine nucleotide exchange factors such as SOS-1 or CDC25 to the cell membrane. The guanine nucleotide exchange factor is now capable of interacting with Ras proteins at the cell membrane to promote a conformational change and the exchange of GDP for GTP bound to Ras. Multiple Ras isoforms have been described, including K-Ras, N-Ras, and H-Ras. Termination of Ras activation occurs upon hydrolysis of RasGTP to RasGDP. Ras proteins have intrinsically low GTPase activity. Thus, the GTPase activity is stimulated by GTPase-activating proteins such as NF-1 GTPase-activating protein/neurofibromin and p120 GTPase activating protein thereby preventing prolonged Ras stimulated signaling. Ras activation is the first step in activation of the MAPK cascade. Following Ras activation, Raf (A-Raf, B-Raf, or Raf-1) is recruited to the cell membrane through binding to Ras and activated in a complex process involving phosphorylation and multiple cofactors that is not completely understood. Raf proteins directly activate MEK1 and MEK2 via phosphorylation of multiple serine residues. MEK1 and MEK2 are themselves tyrosine and threonine/serine dual-specificity kinases that subsequently phosphorylate threonine and tyrosine residues in Erk1 and Erk2 resulting in activation. Although MEK1/2 have no known targets besides Erk proteins, Erk has multiple targets including Elk-1, c-Ets1, c-Ets2, p90RSK1, MNK1, MNK2, and TOB. The cellular functions of Erk are diverse and include regulation of cell proliferation, survival, mitosis, and migration. McCubrey, J. Roles of the Raf/MEK/ERK pathway in cell growth, malignant transformation and drug resistance. Biochimica et Biophysica Acta. 2007; 1773: 1263-1284, hereby fully incorporated by reference in its entirety for all purposes, Friday and Adjei, Clinical Cancer Research (2008) 14, p342-346.

c-Jun N-terminal kinase (INK)/stress-activated protein kinase (SAPK) Pathway: The c-Jun N-terminal kinases (JNKs) were initially described as a family of serine/threonine protein kinases, activated by a range of stress stimuli and able to phosphorylate the N-terminal transactivation domain of the c-Jun transcription factor. This phosphorylation enhances c-Jun dependent transcriptional events in mammalian cells. Further research has revealed three JNK genes (JNK1, JNK2 and JNK3) and their splice-forms as well as the range of external stimuli that lead to JNK activation. JNK1 and JNK2 are ubiquitous, whereas JNK3 is relatively restricted to brain. The predominant MAP2Ks upstream of JNK are MEK4 (MKK4) and MEK7 (MKK7). MAP3Ks with the capacity to activate JNK/SAPKs include MEKKs (MEKK1, -2, -3 and -4), mixed lineage kinases (MLKs, including MLK1-3 and DLK), Tp12, ASKs, TAOs and TAK1. Knockout studies in several organisms indicate that different MAP3Ks predominate in JNK/SAPK activation in response to different upstream stimuli. The wiring may be comparable to, but perhaps even more complex than, MAP3K selection and control of the ERK1/2 pathway. JNK/SAPKs are activated in response to inflammatory cytokines; environmental stresses, such as heat shock, ionizing radiation, oxidant stress and DNA damage; DNA and protein synthesis inhibition; and growth factors. JNKs phosphorylate transcription factors c-Jun, ATF-2, p53, Elk-1, and nuclear factor of activated T cells (NFAT), which in turn regulate the expression of specific sets of genes to mediate cell proliferation, differentiation or apoptosis. JNK proteins are involved in cytokine production, the inflammatory response, stress-induced and developmentally programmed apoptosis, actin reorganization, cell transformation and metabolism. Raman, M. Differential regulation and properties of MAPKs. Oncogene. 2007; 26: 3100-3112, hereby fully incorporated by reference in its entirety for all purposes.

p38 MAPK Pathway: Several independent groups identified the p38 Map kinases, and four p38 family members have been described (α, β, γ, δ). Although the p38 isoforms share about 40% sequence identity with other MAPKs, they share only about 60% identity among themselves, suggesting highly diverse functions. p38 MAPKs respond to a wide range of extracellular cues particularly cellular stressors such as UV radiation, osmotic shock, hypoxia, pro-inflammatory cytokines and less often growth factors. Responding to osmotic shock might be viewed as one of the oldest functions of this pathway, because yeast p38 activates both short and long-term homeostatic mechanisms to osmotic stress. p38 is activated via dual phosphorylation on the TGY motif within its activation loop by its upstream protein kinases MEK3 and MEK6. MEK3/6 are activated by numerous MAP3Ks including MEKK1-4, TAOs, TAK and ASK. p38 MAPK is generally considered to be the most promising MAPK therapeutic target for rheumatoid arthritis as p38 MAPK isoforms have been implicated in the regulation of many of the processes, such as migration and accumulation of leucocytes, production of cytokines and pro-inflammatory mediators and angiogenesis, that promote disease pathogenesis. Further, the p38 MAPK pathway plays a role in cancer, heart and neurodegenerative diseases and may serve as promising therapeutic target. Cuenda, A. p38 MAP-Kinases pathway regulation, function, and role in human diseases. Biochimica et Biophysica Acta. 2007; 1773: 1358-1375; Thalhamer et al., Rheumatology 2008; 47:409-414; Roux, P. ERK and p38 MAPK-Activated Protein Kinases: a Family of Protein Kinases with Diverse Biological Functions. Microbiology and Molecular Biology Reviews. June, 2004; 320-344 hereby fully incorporated by reference in its entirety for all purposes.

Src Family Kinases: Src is the most widely studied member of the largest family of nonreceptor protein tyrosine kinases, known as the Src family kinases (SFKs). Other SFK members include Lyn, Fyn, Lck, Hck, Fgr, Blk, Yrk, and Yes. The Src kinases can be grouped into two sub-categories, those that are ubiquitously expressed (Src, Fyn, and Yes), and those which are found primarily in hematopoietic cells (Lyn, Lck, Hck, Blk, Fgr). (Benati, D. Src Family Kinases as Potential Therapeutic Targets for Malignancies and Immunological Disorders. Current Medicinal Chemistry. 2008; 15: 1154-1165) SFKs are key messengers in many cellular pathways, including those involved in regulating proliferation, differentiation, survival, motility, and angiogenesis. The activity of SFKs is highly regulated intramolecularly by interactions between the SH2 and SH3 domains and intermolecularly by association with cytoplasmic molecules. This latter activation may be mediated by focal adhesion kinase (FAK) or its molecular partner Crk-associated substrate (CAS), which play a prominent role in integrin signaling, and by ligand activation of cell surface receptors, e.g. epidermal growth factor receptor (EGFR). These interactions disrupt intramolecular interactions within Src, leading to an open conformation that enables the protein to interact with potential substrates and downstream signaling molecules. Src can also be activated by dephosphorylation of tyrosine residue Y530. Maximal Src activation requires the autophosphorylation of tyrosine residue Y419 (in the human protein) present within the catalytic domain. Elevated Src activity may be caused by increased transcription or by deregulation due to overexpression of upstream growth factor receptors such as EGFR, HER2, platelet-derived growth factor receptor (PDGFR), fibroblast growth factor receptor (FGFR), vascular endothelial growth factor receptor, ephrins, integrin, or FAK. Alternatively, some human tumors show reduced expression of the negative Src regulator, Csk. Increased levels, increased activity, and genetic abnormalities of Src kinases have been implicated in both solid tumor development and leukemias. Ingley, E. Src family kinases: Regulation of their activities, levels and identification of new pathways. Biochimica et Biophysica Acta. 2008; 1784 56-65, hereby fully incorporated by reference in its entirety for all purposes. Benati and Baldari., Curr Med. Chem. 2008; 15(12):1154-65, Finn (2008) Ann Oncol. May 16, hereby fully incorporated by reference in its entirety for all purposes.

Janus kinase (JAK)/Signal transducers and activators of transcription (STAT) pathway: The JAK/STAT pathway plays a crucial role in mediating the signals from a diverse spectrum of cytokine receptors, growth factor receptors, and G-protein-coupled receptors. Signal transducers and activators of transcription (STAT) proteins play a crucial role in mediating the signals from a diverse spectrum of cytokine receptors growth factor receptors, and G-protein-coupled receptors. STAT directly links cytokine receptor stimulation to gene transcription by acting as both a cytosolic messenger and nuclear transcription factor. In the Janus Kinase (JAK)-STAT pathway, receptor dimerization by ligand binding results in JAK family kinase (JFK) activation and subsequent tyrosine phosphorylation of the receptor, which leads to the recruitment of STAT through the SH2 domain, and the phosphorylation of conserved tyrosine residue. Tyrosine phosphorylated STAT forms a dimer, translocates to the nucleus, and binds to specific DNA elements to activate target gene transcription, which leads to the regulation of cellular proliferation, differentiation, and apoptosis. The entire process is tightly regulated at multiple levels by protein tyrosine phosphatases, suppressors of cytokine signaling and protein inhibitors of activated STAT. In mammals seven members of the STAT family (STAT1, STAT2, STAT3, STAT4, STAT5a, STAT5b and STATE) have been identified. JAKs contain two symmetrical kinase-like domains; the C-terminal JAK homology 1 (JH1) domain possesses tyrosine kinase function while the immediately adjacent JH2 domain is enzymatically inert but is believed to regulate the activity of JH1. There are four JAK family members: JAK1, JAK2, JAK3 and tyrosine kinase 2 (Tyk2). Expression is ubiquitous for JAK1, JAK2 and TYK2 but restricted to hematopoietic cells for JAK3. Mutations in JAK proteins have been described for several myeloid malignancies. Specific examples include but are not limited to: Somatic JAK3 (e.g. JAK3A572V, JAK3V722I, JAK3P132T) and fusion JAK2 (e.g. ETV6-JAK2, PCM1-JAK2, BCR-JAK2) mutations have respectively been described in acute megakaryocytic leukemia and acute leukemia/chronic myeloid malignancies, JAK2 (V617F, JAK2 exon 12 mutations) and MPL MPLW515L/K/S, MPLS505N) mutations associated with myeloproliferative disorders and myeloproliferative neoplasms. JAK2 mutations, primarily JAK2V617F, are invariably associated with polycythemia vera (PV). This mutation also occurs in the majority of patients with essential thrombocythemia (ET) or primary myelofibrosis (PMF) (Tefferi n., Leukemia & Lymphoma, March 2008; 49(3): 388-397). STATs can be activated in a JAK-independent manner by src family kinase members and by oncogenic FLt3 ligand-ITD (Hayakawa and Naoe, Ann N Y Acad. Sci. 2006 November; 1086:213-22; Choudhary et al. Activation mechanisms of STAT5 by oncogenic FLt3 ligand-ITD. Blood (2007) vol. 110 (1) pp. 370-4). Although mutations of STATs have not been described in human tumors, the activity of several members of the family, such as STAT1, STAT3 and STAT5, is dysregulated in a variety of human tumors and leukemias. STAT3 and STAT5 acquire oncogenic potential through constitutive phosphorylation on tyrosine, and their activity has been shown to be required to sustain a transformed phenotype. This was shown in lung cancer where tyrosine phosphorylation of STAT3 was JAK-independent and mediated by EGF receptor activated through mutation and Src. (Alvarez et al., Cancer Research, Cancer Res 2006; 66) STAT5 phosphorylation was also shown to be required for the long-term maintenance of leukemic stem cells. (Schepers et al. STAT5 is required for long-term maintenance of normal and leukemic human stem/progenitor cells. Blood (2007) vol. 110 (8) pp. 2880-2888) In contrast to STAT3 and STAT5, STAT1 negatively regulates cell proliferation and angiogenesis and thereby inhibits tumor formation. Consistent with its tumor suppressive properties, STAT1 and its downstream targets have been shown to be reduced in a variety of human tumors (Rawlings, J. The JAK/STAT signaling pathway. J of Cell Science. 2004; 117 (8):1281-1283, hereby fully incorporated by reference in its entirety for all purposes).

### Drug Transporters

In some embodiments, the present invention provides methods for classification, diagnosis, prognosis of a condition and/or prediction of outcome after administering a therapeutic agent to treat the condition by determining a drug transporter expression and/or function. In some embodiments, the present invention provides methods for classification, diagnosis, prognosis of disease and/or prediction of outcome after administering a therapeutic agent to treat the condition by determining a drug transporter expression and/or function and by characterizing one or more pathways in a population of cells. In some embodiments, the therapeutic agent is a drug transporter substrate.

A key issue in the treatment of many cancers is the development of resistance to chemotherapeutic drugs. Of the many resistance mechanisms, two classes of transporters play a major role. The human ATP-binding cassette (ABC) superfamily of proteins consists of 49 membrane proteins that transport a diverse array of substrates, including sugars, amino acids, bile salts lipids, sterols, nucleotides, endogenous metabolites, ions, antibiotics drugs and toxins out of cells using the energy of hydrolysis of ATP. ATP-binding-cassette (ABC) transporters are evolutionary extremely well-conserved transmembrane proteins that are highly expressed in hematopoietic stem cells (HSCs). The physiological function in human stem cells is believed to be protection against genetic damage caused by both environmental and naturally occurring xenobiotics. Additionally, ABC transporters have been implicated in the maintenance of quiescence and cell fate decisions of stem cells. These physiological roles suggest a potential role in the pathogenesis and biology of stem cell-derived hematological malignancies such as acute and chronic myeloid leukemia (Raaijmakers, Leukemia (2007) 21, 2094-2102, Zhou et al., Nature Medicine, 2001, 7, p 1028-1034

Several ABC proteins are multidrug efflux pumps that not only protect the body from exogenous toxins, but also play a role in uptake and distribution of therapeutic drugs. Expression of these proteins in target tissues causes resistance to treatment with multiple drugs. (Gillet et al., Biochimica et Biophysica Acta (2007) 1775, p 237, Sharom (2008) Pharmacogenomics 9 p 105). A more detailed discussion of the ABC family members with critical roles in resistance and poor outcome to treatment is discussed below

The second class of plasma membrane transporter proteins that play a role in the uptake of nucleoside-derived drugs are the Concentrative and Equilibrative Nucleoside Transporters (CNT and ENT, respectively), encoded by gene families SLC28 and SLC29 (Pastor-Anglada (2007) J. Physiol. Biochem 63, p 97). They mediate the uptake of natural nucleosides and a variety of nucleoside-derived drugs, mostly used in anti-cancer therapy. In vitro studies, have shown that one mechanism of nucleoside resistance can be mediated through mutations in the gene for ENT1/SLC29A1 resulting in lack of detectable protein (Cai et al., Cancer Research (2008) 68, p 2349). Studies have also described in vivo mechanisms of resistance to nucleoside analogues involving low or non-detectable levels of ENT1 in Acute Myeloid Leukemia (AML), Mantle Cell lymphoma and other leukemias (Marce et al., Malignant Lymphomas (2006), 91, p 895).

Of the ABC transporter family, three family members account for most of the multiple drug resistance (MDR) in humans; P-glycoprotein (Pgp/MDR1/ABCB1), MDR associated protein (MRP1, ABCC1) and breast cancer resistance protein (BCRP, ABCG2 or MXR). Pgp/MDR1 and ABCG2 can export both unmodified drugs and drug conjugates, whereas MRP1 exports glutathione and other drug conjugates as well as unconjugated drugs together with free glutathione. All three ABC transporters demonstrate export activity for a broad range of structurally unrelated drugs and display both distinct and overlapping specificities. For example, MRP1 promotes efflux of drug-glutathione conjugates, vinca alkaloids, camptothecin, but not taxol. Examples of drugs exported by ABCG2 include mitoxantrone, etoposide, daunorubicin as well as the tyrosine kinase inhibitors Gleevec and Iressa. In treatment regimens for leukemias, one of the main obstacles to achieving remission is intrinsic and acquired resistance to chemotherapy mediated by the ABC drug transporters. Several reports have described correlations between transporter expression levels as well as their function, evaluated through the use of fluorescent dyes, with resistance of patients to chemotherapy regimens. Notably, in AML, studies have shown that expression of Pgp/MDR1 is associated with a lower rate of complete response to induction chemotherapy and a higher rate of resistant disease in both elderly and younger AML patients (Leith et al., Blood (1997) 89 p 3323, Leith et al., Blood (1999) 94, p 1086). Legrand et al., (Blood (1998) 91, p 4480) showed that Pgp/MDR1 and MRP1 function in CD34+ blast cells are negative prognostic factors in AML and further, the same group showed that a high level of simultaneous activity of Pgp/MDR1 and MRP1 was predictive of poor treatment outcome (Legrand et al., (Blood (1999) 94, p 1046). In two more recent studies, elevated expression of Pgp/MDR1 and BCRP in CD34+/CD38− AML subpopulations were found in 8 out of 10 non-responders as compared to 0 out of 10 in responders to induction chemotherapy (Ho et al., Experimental Hematology (2008) 36, p 433). In a second study, evaluation of Pgp/MDR1, MRP1, BCRP/ABCG2 and lung resistance protein showed that the more immature subsets of leukemic stem cells expressed higher levels of these proteins compared more mature leukemic subsets (Figueiredo-Pontes et al., Clinical Cytometry (2008) 74B p 163).

Experimentally, it is possible to correlate expression of transporter proteins with their function by the use of inhibitors including but not limited to cyclosporine (measures Pgp function), probenecid (measures MRP1 function), fumitremorgin C, and a derivative Ko143, reserpine (measures ABCG2 function). Although these molecules inhibit a variety of transporters, they do permit some correlations to be made between protein expression and function (Legrand et al., (Blood (1998) 91, p 4480), Legrand et al., (Blood (1999) 94, p 1046, Zhou et al., Nature Medicine, 2001, 7, p 1028-1034, Sarkardi et al., Physiol Rev 2006 86: 1179-1236).

Extending the use of these inhibitors, they can be used to make correlations within subpopulations of cells gated both for phenotypic markers denoting stages of development along hematopoietic and lymphoid lineages, as well as reagents that recognize the transporter proteins themselves. Thus it will be possible to simultaneously measure protein expression and function

### DNA Damage and Apoptosis

The response to DNA damage is a protective measure taken by cells to prevent or delay genetic instability and tumorigenesis. It allows cells to undergo cell cycle arrest and gives them an opportunity to either: repair the damaged DNA and resume passage through the cell cycle or, if the damage is irreparable, trigger senescence or an apoptotic program leading to cell death (Wade Harper et al., Molecular Cell, (2007) 28 p 739±745, Bartek J et al., Oncogene (2007)26 p 7773-9).

Several protein complexes are positioned at strategic points within the DNA damage response pathway and act as sensors of DNA damage, or transducers or effectors of a DNA damage response. Depending on the nature of DNA damage for example; double stranded breaks, single strand breaks, single base alterations due to alkylation, oxidation etc, there is an assembly of specific DNA damage sensor protein complexes in which activated ataxia telangiectasia mutated (ATM) and ATM- and Rad3 related (ATR) kinases phosphorylate and subsequently activate the checkpoint kinases Chk1 and Chk2. Both of these DNA-signal transducer kinases amplify the damage response by phosphorylating a multitude of substrates. Both checkpoint kinases have overlapping and distinct roles in orchestrating the cell's response to DNA damage.

Activation of Chk2 kinase activity involves ATM mediated phosphorylation of threonine 68 and homo-dimerization (Reinhardt H C, Yaffe M B Curr Opin Cell Biol. 2009 April; 21(2):245-55, Antoni L, Sodha N, Collins I, Garrett M D Nat Rev Cancer. 2007 December; 7(12):925-36. This in turn initiates the DNA repair process of which there are at least twelve distinct mechanisms. The choice of which repair process to use depends on the type of lesion and on the cell-cycle phase of the cell. For example, a DNA double-strand break (DSB) in S and G2 phases is readily repaired by homologous recombination (Branzei and Foiani Nat Rev Mol Cell Biol. 2008 April; 9(4):297-308). If DNA repair is successful cell cycle progression is resumed (Antoni et al., Nature reviews cancer (2007) 7, p 925-936).

When DNA repair is no longer possible, the cell undergoes apoptosis mediated by Chk-2 through p53 independent and dependent pathways. Chk2 substrates that operate in a p53-independent manner include the E2F1 transcription factor, the tumor suppressor promyelocytic leukemia (PML) and the polo-like kinases 1 and 3 (PLK1 and PLK3). E2F1 drives the expression of a number of apoptotic genes including caspases 3, 7, 8 and 9 as well as the pro-apoptotic Bcl-2 related proteins (Bim, Noxa, PUMA).

In its response to DNA damage, p53 activates the transcription of a program of genes that regulate DNA repair, cell cycle arrest, senescence and apoptosis. The overall functions of p53 are to preserve fidelity in DNA replication such that when cell division occurs tumorigenic potential can be avoided. In such a role, p53 is described as “The Guardian of the Genome (Riley et al., Nature Reviews Molecular Cell Biology (2008) 9 p402-412). The diverse alarm signals that impinge on p53 result in a rapid increase in its levels through a variety of post translational modifications. Worthy of mention is the phosphorylation of amino acid residues within the amino terminal portion of p53 such that p53 is no longer under the regulation of Mdm2 The responsible kinases are ATM, Chk1 and Chk2. The subsequent stabilization of p53 permits it to transcriptionally regulate multiple pro-apoptotic members of the Bcl-2 family, including Bax, Bid, Puma, and Noxa (Discussion below).

The series of events that are mediated by p53 to promote apoptosis including DNA damage, anoxia and imbalances in growth-promoting signals are sometimes termed the ‘intrinsic apoptotic” program since the signals triggering it originate within the cell. An alternate route of activating the apoptotic pathway can occur from the outside of the cell mediated by the binding of ligands to transmembrane death receptors. This extrinsic or receptor mediated apoptotic program acting through their receptor death domains eventually converges on the intrinsic, mitochondrial apoptotic pathway as discussed below (Sprick et al., Biochim Biophys Acta. (2004) 1644 p 125-32).

Key regulators of apoptosis are proteins of the Bcl-2 family. The founding member, the Bcl-2 proto-oncogene was first identified at the chromosomal breakpoint of t(14:18) bearing human follicular B cell lymphoma. Unexpectedly, expression of Bcl-2 was proved to block rather than promote cell death following multiple pathological and physiological stimuli (Danial and Korsemeyer, Cell (2204) 116, p 205-219). The Bcl-2 family has at least 20 members which are key regulators of apoptosis, functioning to control mitochondrial permeability as well as the release of proteins important in the apoptotic program. The ratio of anti- to pro-apoptotic molecules such as Bcl-2/Bax constitutes a rheostat that sets the threshold of susceptibility to apoptosis for the intrinsic pathway, which utilizes organelles such as the mitochondrion to amplify death signals. The family can be divided into 3 subclasses based on structure and impact on apoptosis. Family members of subclass 1 including Bcl-2, Bcl-X_{L} and Mcl-1 are characterized by the presence of 4 Bcl-2 homology domains (BH1, BH2, BH3 and BH4) and are anti-apoptotic. The structure of the second subclass members is marked for containing 3 BH domains and family members such as Bax and Bak possess pro-apoptotic activities. The third subclass, termed the BH3-only proteins include Noxa, Puma, Bid, Bad and Bim. They function to promote apoptosis either by activating the pro-apoptotic members of group 2 or by inhibiting the anti-apoptotic members of subclass 1 (Er et al., Biochimica et Biophysica Act (2006) 1757, p 1301-1311, Fernandez-Luna Cellular Signaling (2008) Advance Publication Online).

The role of mitochondria in the apoptotic process was clarified as involving an apoptotic stimulus resulting in depolarization of the outer mitochondrial membrane leading to a leak of cytochrome C into the cytoplasm. Association of cytochrome C molecules with adaptor apoptotic protease activating factor (APAF) forms a structure called the apoptosome which can activate enzymatically latent procaspase 9 into a cleaved activated form. Caspase 9 is one member of a family of cysteine aspartyl-specific proteases; genes encoding 11 of these proteases have been mapped in the human genome. Activated caspase 9, classified as an intiator caspase, then cleaves procaspase 3 which cleaves more downstream procaspases, classified as executioner caspases, resulting in an amplification cascade that promotes cleavage of death substrates including poly(ADP-ribose) polymerase 1 (PARP). The cleavage of PARP produces 2 fragments both of which have a role in apoptosis (Soldani and Scovassi Apoptosis (2002) 7, p 321). A further level of apoptotic regulation is provided by smac/Diablo, a mitochondrial protein that inactivates a group of anti-apoptotic proteins termed inhibitors of apoptosis (IAPs) (Huang et al., Cancer Cell (2004) 5 p 1-2). IAPs operate to block caspase activity in 2 ways; they bind directly to and inhibit caspase activity and in certain cases they can mark caspases for ubiquitination and degradation.

The balance of pro- and anti-apoptotic proteins is tightly regulated under normal physiological conditions. Tipping of this balance either way results in disease. An oncogenic outcome results from the inability of tumor cells to undergo apoptosis and this can be caused by over-expression of anti-apoptotic proteins or reduced expression or activity of pro-apoptotic protein

Interrogation of the apoptotic machinery will also be performed with a combination of Cytarabine and Daunorubicin at clinically relevant concentrations based on peak plasma drug levels. The standard dose of Cytarabine, 100 mg/m2, yields a peak plasma concentration of approximately 40 nM, whereas high dose Cytarabine, 3 g/m2, yields a peak plasma concentration of 2 uM. Daunorubicin at 25 mg/m2 yields a peak plasma concentration of 50 ng/ml and at 50 mg/m2 yields a peak plasma concentration of 200 ng/ml. Our in vitro apoptosis assay will use concentrations of Cytarabine up to 2 uM, and concentrations of Daunorubicin up to 200 ng/ml.

### Cell Cycle

The cell cycle, or cell-division cycle, is the series of events that take place in a cell leading to its division and duplication (replication). The cell cycle consists of five distinct phases: G0 phase, G1 phase, S (synthesis) phase, G2 phase (these four phases are collectively known as interphase) and M phase (mitosis). M phase is itself composed of two tightly coupled processes: mitosis, in which the cell's chromosomes are divided between two daughter cells, and cytokinesis, in which the cell's cytoplasm divides forming distinct cells. Activation of each of the five cell cycle phases is dependent on the proper progression and completion of the previous one. Cells that have temporarily or reversibly stopped dividing are said to have entered a state of quiescence called G0 phase.

Regulation of the cell cycle involves processes crucial to the survival of a cell, including the detection and repair of genetic damage as well as the prevention of uncontrolled cell division. The molecular events that control the cell cycle are ordered and directional; that is, each process occurs in a sequential fashion and it is impossible to “reverse” the cycle.

Two key classes of regulatory molecules, cyclins and cyclin-dependent kinases (CDKs), determine a cell's progress through the cell cycle. Many of the genes encoding cyclins and CDKs are conserved among all eukaryotes, but in general more complex organisms have more elaborate cell cycle control systems that incorporate more individual components. Many of the relevant genes were first identified by studying yeast, especially Saccharomyces cerevisiae genetic nomenclature in yeast dubs many these genes cdc (for “cell division cycle”) followed by an identifying number, e.g., cdc25.

Cyclins form the regulatory subunits and CDKs the catalytic subunits of an activated heterodimer; cyclins have no catalytic activity and CDKs are inactive in the absence of a partner cyclin. When activated by a bound cyclin, CDKs perform a common biochemical reaction called phosphorylation that activates or inactivates target proteins to orchestrate coordinated entry into the next phase of the cell cycle. Different cyclin-CDK combinations determine the downstream proteins targeted. CDKs are constitutively expressed in cells whereas cyclins are synthesized at specific stages of the cell cycle, in response to various molecular signals.

Upon receiving a pro-mitotic extracellular signal, G1 cyclin-CDK complexes become active to prepare the cell for S phase, promoting the expression of transcription factors that in turn promote the expression of S cyclins and of enzymes required for DNA replication. The G1 cyclin-CDK complexes also promote the degradation of molecules that function as S phase inhibitors by targeting them for ubiquitination. Once a protein has been ubiquitinated, it is targeted for proteolytic degradation by the proteasome. Active S cyclin-CDK complexes phosphorylate proteins that make up the pre-replication complexes assembled during G1 phase on DNA replication origins. The phosphorylation serves two purposes: to activate each already-assembled pre-replication complex, and to prevent new complexes from forming. This ensures that every portion of the cell's genome will be replicated once and only once. The reason for prevention of gaps in replication is fairly clear, because daughter cells that are missing all or part of crucial genes will die. However, for reasons related to gene copy number effects, possession of extra copies of certain genes would also prove deleterious to the daughter cells.

Mitotic cyclin-CDK complexes, which are synthesized but inactivated during S and G2 phases, promote the initiation of mitosis by stimulating downstream proteins involved in chromosome condensation and mitotic spindle assembly. A critical complex activated during this process is an ubiquitin ligase known as the anaphase-promoting complex (APC), which promotes degradation of structural proteins associated with the chromosomal kinetochore. APC also targets the mitotic cyclins for degradation, ensuring that telophase and cytokinesis can proceed. Interphase: Interphase generally lasts at least 12 to 24 hours in mammalian tissue. During this period, the cell is constantly synthesizing RNA, producing protein and growing in size. By studying molecular events in cells, scientists have determined that interphase can be divided into 4 steps: Gap 0 (G0), Gap 1 (G1), S (synthesis) phase, Gap 2 (G2).

Cyclin D is the first cyclin produced in the cell cycle, in response to extracellular signals (e.g. growth factors). Cyclin D binds to existing CDK4, forming the active cyclin D-CDK4 complex. Cyclin D-CDK4 complex in turn phosphorylates the retinoblastoma susceptibility protein (Rb). The hyperphosphorylated Rb dissociates from the E2F/DP1/Rb complex (which was bound to the E2F responsive genes, effectively “blocking” them from transcription), activating E2F. Activation of E2F results in transcription of various genes like cyclin E, cyclin A, DNA polymerase, thymidine kinase, etc. Cyclin E thus produced binds to CDK2, forming the cyclin E-CDK2 complex, which pushes the cell from G1 to S phase (G1/S transition). Cyclin B along with cdc2 (cdc2-fission yeasts (CDK1-mammalia)) forms the cyclin B-cdc2 complex, which initiates the G2/M transition. Cyclin B-cdc2 complex activation causes breakdown of nuclear envelope and initiation of prophase, and subsequently, its deactivation causes the cell to exit mitosis.

Two families of genes, the Cip/Kip family and the INK4a/ARF (Inhibitor of Kinase 4/Alternative Reading Frame) prevent the progression of the cell cycle. Because these genes are instrumental in prevention of tumor formation, they are known as tumor suppressors.

The Cip/Kip family includes the genes p21, p27 and p57. They halt cell cycle in G1 phase, by binding to, and inactivating, cyclin-CDK complexes. p21 is a p53 response gene (which, in turn, is triggered by DNA damage, e.g. due to radiation). p27 is activated by Transforming Growth Factor β (TGF β), a growth inhibitor.

The INK4a/ARF family includes p161NK4a, which binds to CDK4 and arrests the cell cycle in G1 phase, and p14arf which prevents p53 degradation.

Cell cycle checkpoints are used by the cell to monitor and regulate the progress of the cell cycle. Checkpoints prevent cell cycle progression at specific points, allowing verification of necessary phase processes and repair of DNA damage. The cell cannot proceed to the next phase until checkpoint requirements have been met.

Several checkpoints are designed to ensure that damaged or incompletely synthesized DNA is not passed on to daughter cells. Two main checkpoints exist: the G1/S checkpoint and the G2/M checkpoint. G1/S transition is a rate-limiting step in the cell cycle and is also known as restriction point. An alternative model of the cell cycle response to DNA damage has also been proposed, known as the postreplication checkpoint. p53 plays an important role in triggering the control mechanisms at both G1/S and G2/M checkpoints.

DAPI (4′,6-Diamidino-2-phenylindole) is a blue fluorescent probe that fluoresces brightly when it is selectively bound to the minor groove of double stranded DNA where its fluorescence is approximately 20-fold greater than in the non-bound state. DAPI has an excitation maximum at 345 nm and an emission maximum at 455 nm. Cells stained with DAPI emit fluorescence in direct proportion to their DNA content. An exponentially growing population of cells will have a DNA content distribution containing an initial peak of G0/G1 cells, a valley of S Phase cells, and a second peak containing G2/M cells. Cells in the G2/M Phase have twice the DNA content as cells in the G0/G1 Phase. DAPI offers a rapid method for measuring the DNA content of cells and provides a convenient research tool to monitor cell cycle status and regulation.

In some embodiments, the kits of the present invention comprise one or binding elements to measure one or more activatable elements within a cell cycle pathway in response to a modulator that slows or stops the growth of cells and/or induces apoptosis of cells. In some embodiments, the kits further comprise the modulator that slows or stops the growth of cells and/or induces apoptosis of cells. In some embodiments, the activatable element is selected from the group consisting of, Cdk1, Cyclin B1, Histone H3, Cyclin D1, p15, p16, and p21. In some embodiments, the modulator that slows or arrests cell cycle progression, and/or induces apoptosis of cells is selected from the group consisting of Staurosporine, Etoposide, Mylotarg, Daunorubicin, Idarubicin and analogs (idarubicin, epirubicin), Ara-C, Vidaza, Mitoxantrone, Clofarabine, Cladribine, Dacogen, HydroxyUrea, and Zolinza.

### Modulators

In some embodiments, the methods and composition utilize a modulator. A modulator can be an activator, a therapeutic agent, an inhibitor or a compound capable of impacting a cellular pathway. Modulators can also take the form of environmental cues and inputs.

Modulation can be performed in a variety of environments. In some embodiments, cells are exposed to a modulator immediately after collection. In some embodiments where there is a mixed population of cells, purification of cells is performed after modulation. In some embodiments, whole blood is collected to which a modulator is added. In some embodiments, cells are modulated after cells have been isolated. As an illustrative example, whole blood can be collected and processed for an enriched fraction of lymphocytes that is then exposed to a modulator. Modulation can include exposing cells to more than one modulator. For instance, in some embodiments, cells are exposed to at least 2, 3, 4, 5, 6, 7, 8, 9, or 10 modulators. See U.S. Patent Application 61/048,657 which is incorporated by reference.

In some embodiments, cells are cultured post collection in a suitable media before exposure to a modulator. In some embodiments, the media is a growth media. In some embodiments, the growth media is a complex media that may include serum. In some embodiments, the growth media comprises serum. In some embodiments, the serum is selected from the group consisting of fetal bovine serum, bovine serum, human serum, porcine serum, horse serum, and goat serum. In some embodiments, the serum level ranges from 0.0001% to 30%. In some embodiments, the growth media is a chemically defined minimal media and is without serum. In some embodiments, cells are cultured in a differentiating media.

Modulators include chemical and biological entities, and physical or environmental stimuli. Modulators can act extracellularly or intracellularly. Chemical and biological modulators include growth factors, cytokines, chemokines, drugs, immune modulators, ions, neurotransmitters, adhesion molecules, hormones, small molecules, inorganic compounds, polynucleotides, antibodies, natural compounds, lectins, lactones, chemotherapeutic agents, biological response modifiers, carbohydrate, proteases and free radicals. Modulators include complex and undefined biologic compositions that may comprise cellular or botanical extracts, cellular or glandular secretions, physiologic fluids such as serum, amniotic fluid, or venom. Physical and environmental stimuli include electromagnetic, ultraviolet, infrared or particulate radiation, redox potential and pH, the presence or absences of nutrients, changes in temperature, changes in oxygen partial pressure, changes in ion concentrations and the application of oxidative stress. Modulators can be endogenous or exogenous and may produce different effects depending on the concentration and duration of exposure to the single cells or whether they are used in combination or sequentially with other modulators. Modulators can act directly on the activatable elements or indirectly through the interaction with one or more intermediary biomolecule. Indirect modulation includes alterations of gene expression wherein the expressed gene product is the activatable element or is a modulator of the activatable element.

In some embodiments, the modulator is an activator. In some embodiments the modulator is an inhibitor. In some embodiments, cells are exposed to one or more modulators. In some embodiments, cells are exposed to at least 2, 3, 4, 5, 6, 7, 8, 9, or 10 modulators. In some embodiments, cells are exposed to at least two modulators, wherein one modulator is an activator and one modulator is an inhibitor. In some embodiments, cells are exposed to at least 2, 3, 4, 5, 6, 7, 8, 9, or 10 modulators, where at least one of the modulators is an inhibitor.

In some embodiments, the cross-linker is a molecular binding entity. In some embodiments, the molecular binding entityis a monovalent, bivalent, or multivalent is made more multivalent by attachment to a solid surface or tethered on a nanoparticle surface to increase the local valency of the epitope binding domain.

In some embodiments, the inhibitor is an inhibitor of a cellular factor or a plurality of factors that participates in a cellular pathway (e.g. signaling cascade) in the cell. In some embodiments, the inhibitor is a phosphatase inhibitor.

In some embodiments, the activation level of an activatable element in a cell is determined by contacting the cell with an inhibitor and a modulator, where the modulator can be an inhibitor or an activator. In some embodiments, the activation level of an activatable element in a cell is determined by contacting the cell with an inhibitor and an activator. In some embodiments, the activation level of an activatable element in a cell is determined by contacting the cell with two or more modulators.

### Detection

In practicing the methods of this invention, the detection of the status of the one or more activatable elements can be carried out by a person, such as a technician in the laboratory. Alternatively, the detection of the status of the one or more activatable elements can be carried out using automated systems. In either case, the detection of the status of the one or more activatable elements for use according to the methods of this invention is performed according to standard techniques and protocols well-established in the art.

One or more activatable elements can be detected and/or quantified by any method that detects and/or quantitates the presence of the activatable element of interest. Such methods may include radioimmunoassay (RIA) or enzyme linked immunoabsorbance assay (ELISA), immunohistochemistry, immunofluorescent histochemistry with or without confocal microscopy, reversed phase assays, homogeneous enzyme immunoassays, and related non-enzymatic techniques, Western blots, whole cell staining, immunoelectronmicroscopy, nucleic acid amplification, gene array, protein array, mass spectrometry, patch clamp, 2-dimensional gel electrophoresis, differential display gel electrophoresis, microsphere-based multiplex protein assays, label-free cellular assays and flow cytometry, etc. U.S. Pat. No. 4,568,649 describes ligand detection systems, which employ scintillation counting. These techniques are particularly useful for modified protein parameters. Cell readouts for proteins and other cell determinants can be obtained using fluorescent or otherwise tagged reporter molecules. Flow cytometry methods are useful for measuring intracellular parameters. See the above patents and applications for example methods.

In some embodiments, the present invention provides methods for determining an activatable element's activation profile for a single cell. The methods may comprise analyzing cells by flow cytometry on the basis of the activation level of at least two activatable elements. Binding elements (e.g. activation state-specific antibodies) are used to analyze cells on the basis of activatable element activation level, and can be detected as described below. Alternatively, non-binding elements systems as described above can be used in any system described herein.

Detection of cell signaling states may be accomplished using binding elements and labels. Cell signaling states may be detected by a variety of methods known in the art. They generally involve a binding element, such as an antibody, and a label, such as a fluorchrome to form a detection element. Detection elements do not need to have both of the above agents, but can be one unit that possesses both qualities. These and other methods are well described in U.S. Pat. Nos. 7,381535 and 7,393,656 and U.S. Ser. Nos. 10/193,462; 11/655,785; 11/655,789; 11/655,821; 11/338,957, 61/048,886; 61/048,920; and 61/048,657 which are all incorporated by reference in their entireties.

In one embodiment of the invention, it is advantageous to increase the signal to noise ratio by contacting the cells with the antibody and label for a time greater than 5, 6, 7, 8, 9, 10, 11, 12, 13, 14, 15, 16, 17, 18, 19, 20, 24 or up to 48 or more hours.

When using fluorescent labeled components in the methods and compositions of the present invention, it will recognized that different types of fluorescent monitoring systems, e.g., cytometric measurement device systems, can be used to practice the invention. In some embodiments, flow cytometric systems are used or systems dedicated to high throughput screening, e.g. 96 well or greater microtiter plates. Methods of performing assays on fluorescent materials are well known in the art and are described in, e.g., Lakowicz, J. R., Principles of Fluorescence Spectroscopy, New York: Plenum Press (1983); Herman, B., Resonance energy transfer microscopy, in: Fluorescence Microscopy of Living Cells in Culture, Part B, Methods in Cell Biology, vol. 30, ed. Taylor, D. L. & Wang, Y.-L., San Diego: Academic Press (1989), pp. 219-243; Turro, N. J., Modern Molecular Photochemistry, Menlo Park: Benjamin/Cummings Publishing Col, Inc. (1978), pp. 296-361.

Fluorescence in a sample can be measured using a fluorimeter. In general, excitation radiation, from an excitation source having a first wavelength, passes through excitation optics. The excitation optics cause the excitation radiation to excite the sample. In response, fluorescent proteins in the sample emit radiation that has a wavelength that is different from the excitation wavelength. Collection optics then collect the emission from the sample. The device can include a temperature controller to maintain the sample at a specific temperature while it is being scanned. According to one embodiment, a multi-axis translation stage moves a microtiter plate holding a plurality of samples in order to position different wells to be exposed. The multi-axis translation stage, temperature controller, auto-focusing feature, and electronics associated with imaging and data collection can be managed by an appropriately programmed digital computer. The computer also can transform the data collected during the assay into another format for presentation. In general, known robotic systems and components can be used.

Other methods of detecting fluorescence may also be used, e.g., Quantum dot methods (see, e.g., Goldman et al., J. Am. Chem. Soc. (2002) 124:6378-82; Pathak et al. J. Am. Chem. Soc. (2001) 123:4103-4; and Remade et al., Proc. Natl. Sci. USA (2000) 18:553-8, each expressly incorporated herein by reference) as well as confocal microscopy. In general, flow cytometry involves the passage of individual cells through the path of a laser beam. The scattering the beam and excitation of any fluorescent molecules attached to, or found within, the cell is detected by photomultiplier tubes to create a readable output, e.g. size, granularity, or fluorescent intensity.

The detecting, sorting, or isolating step of the methods of the present invention can entail fluorescence-activated cell sorting (FACS) techniques, where FACS is used to select cells from the population containing a particular surface marker, or the selection step can entail the use of magnetically responsive particles as retrievable supports for target cell capture and/or background removal. A variety of FACS systems are known in the art and can be used in the methods of the invention (see e.g., WO99/54494, filed Apr. 16, 1999; U.S. Ser. No. 20010006787, filed Jul. 5, 2001, each expressly incorporated herein by reference).

In some embodiments, a FACS cell sorter (e.g. a FACSVantage™ Cell Sorter, Becton Dickinson Immunocytometry Systems, San Jose, Calif.) is used to sort and collect cells based on their activation profile (positive cells) in the presence or absence of an increase in activation level in an activatable element in response to a modulator. Other flow cytometers that are commercially available include the LSR II and the Canto II both available from Becton Dickinson. See Shapiro, Howard M., Practical Flow Cytometry, 4th Ed., John Wiley & Sons, Inc., 2003 for additional information on flow cytometers.

In some embodiments, the cells are first contacted with fluorescent-labeled activation state-specific binding elements (e.g. antibodies) directed against specific activation state of specific activatable elements. In such an embodiment, the amount of bound binding element on each cell can be measured by passing droplets containing the cells through the cell sorter. By imparting an electromagnetic charge to droplets containing the positive cells, the cells can be separated from other cells. The positively selected cells can then be harvested in sterile collection vessels. These cell-sorting procedures are described in detail, for example, in the FACSVantage™. Training Manual, with particular reference to sections 3-11 to 3-28 and 10-1 to 10-17, which is hereby incorporated by reference in its entirety. See the patents, applications and articles referred to, and incorporated above for detection systems.

Fluorescent compounds such as Daunorubicin and Enzastaurin are problematic for flow cytometry based biological assays due to their broad fluorescence emission spectra. These compounds get trapped inside cells after fixation with agents like paraformaldehyde, and are excited by one or more of the lasers found on flow cytometers. The fluorescence emission of these compounds is often detected in multiple PMT detectors which complicates their use in multiparametric flow cytometry. A way to get around this problem is to compensate out the fluorescence emission of the compound from the PMT detectors used to measure the relevant biological markers. This is achieved using a PMT detector with a bandpass filter near the emission maximum of the fluorescent compound, and cells incubated with the compound as the compensation control when calculating a compensation matrix. The cells incubated with the fluorescent compound are fixed with paraformaldehyde, then washed and permeabilized with 100% methanol. The methanol is washed out and the cells are mixed with unlabeled fixed/permed cells to yield a compensation control consisting of a mixture of fluorescent and negative cell populations.

In another embodiment, positive cells can be sorted using magnetic separation of cells based on the presence of an isoform of an activatable element. In such separation techniques, cells to be positively selected are first contacted with specific binding element (e.g., an antibody or reagent that binds an isoform of an activatable element). The cells are then contacted with retrievable particles (e.g., magnetically responsive particles) that are coupled with a reagent that binds the specific binding element. The cell-binding element-particle complex can then be physically separated from non-positive or non-labeled cells, for example, using a magnetic field. When using magnetically responsive particles, the positive or labeled cells can be retained in a container using a magnetic field while the negative cells are removed. These and similar separation procedures are described, for example, in the Baxter Immunotherapy Isolex training manual which is hereby incorporated in its entirety.

In some embodiments, methods for the determination of a receptor element activation state profile for a single cell are provided. The methods comprise providing a population of cells and analyze the population of cells by flow cytometry. Preferably, cells are analyzed on the basis of the activation level of at least two activatable elements. In some embodiments, a multiplicity of activatable element activation-state antibodies is used to simultaneously determine the activation level of a multiplicity of elements.

In some embodiment, cell analysis by flow cytometry on the basis of the activation level of at least two elements is combined with a determination of other flow cytometry readable outputs, such as the presence of surface markers, granularity and cell size to provide a correlation between the activation level of a multiplicity of elements and other cell qualities measurable by flow cytometry for single cells.

As will be appreciated, the present invention also provides for the ordering of element clustering events in signal transduction. Particularly, the present invention allows the artisan to construct an element clustering and activation hierarchy based on the correlation of levels of clustering and activation of a multiplicity of elements within single cells. Ordering can be accomplished by comparing the activation level of a cell or cell population with a control at a single time point, or by comparing cells at multiple time points to observe subpopulations arising out of the others.

The present invention provides a valuable method of determining the presence of cellular subsets within cellular populations. Ideally, signal transduction pathways are evaluated in homogeneous cell populations to ensure that variances in signaling between cells do not qualitatively nor quantitatively mask signal transduction events and alterations therein. As the ultimate homogeneous system is the single cell, the present invention allows the individual evaluation of cells to allow true differences to be identified in a significant way.

Thus, the invention provides methods of distinguishing cellular subsets within a larger cellular population. As outlined herein, these cellular subsets often exhibit altered biological characteristics (e.g. activation levels, altered response to modulators) as compared to other subsets within the population. For example, as outlined herein, the methods of the invention allow the identification of subsets of cells from a population such as primary cell populations, e.g. peripheral blood mononuclear cells that exhibit altered responses (e.g. response associated with presence of a condition) as compared to other subsets. In addition, this type of evaluation distinguishes between different activation states, altered responses to modulators, cell lineages, cell differentiation states, etc.

As will be appreciated, these methods provide for the identification of distinct signaling cascades for both artificial and stimulatory conditions in complex cell populations, such as peripheral blood mononuclear cells, or naive and memory lymphocytes.

When necessary cells are dispersed into a single cell suspension, e.g. by enzymatic digestion with a suitable protease, e.g. collagenase, dispase, etc; and the like. An appropriate solution is used for dispersion or suspension. Such solution will generally be a balanced salt solution, e.g. normal saline, PBS, Hanks balanced salt solution, etc., conveniently supplemented with fetal calf serum or other naturally occurring factors, in conjunction with an acceptable buffer at low concentration, generally from 5-25 mM. Convenient buffers include HEPES1 phosphate buffers, lactate buffers, etc. The cells may be fixed, e.g. with 3% paraformaldehyde, and are usually permeabilized, e.g. with ice cold methanol; HEPES-buffered PBS containing 0.1% saponin, 3% BSA; covering for 2 min in acetone at −200 C; and the like as known in the art and according to the methods described herein.

In some embodiments, one or more cells are contained in a well of a 96 well plate or other commercially available multiwell plate. In an alternate embodiment, the reaction mixture or cells are in a cytometric measurement device. Other multiwell plates useful in the present invention include, but are not limited to 384 well plates and 1536 well plates. Still other vessels for containing the reaction mixture or cells and useful in the present invention will be apparent to the skilled artisan.

The addition of the components of the assay for detecting the activation level or activity of an activatable element, or modulation of such activation level or activity, may be sequential or in a predetermined order or grouping under conditions appropriate for the activity that is assayed for. Such conditions are described here and known in the art. Moreover, further guidance is provided below (see, e.g., in the Examples).

In some embodiments, the activation level of an activatable element is measured using Inductively Coupled Plasma Mass Spectrometer (ICP-MS). A binding element that has been labeled with a specific element binds to the activativatable. When the cell is introduced into the ICP, it is atomized and ionized. The elemental composition of the cell, including the labeled binding element that is bound to the activatable element, is measured. The presence and intensity of the signals corresponding to the labels on the binding element indicates the level of the activatable element on that cell (Tanner et al. Spectrochimica Acta Part B: Atomic Spectroscopy, 2007 March; 62(3):188-195.).

As will be appreciated by one of skill in the art, the instant methods and compositions find use in a variety of other assay formats in addition to flow cytometry analysis. For example, DNA microarrays are commercially available through a variety of sources (Affymetrix, Santa Clara Calif.) or they can be custom made in the lab using arrayers which are also know (Perkin Elmer). In addition, protein chips and methods for synthesis are known. These methods and materials may be adapted for the purpose of affixing activation state binding elements to a chip in a prefigured array. In some embodiments, such a chip comprises a multiplicity of element activation state binding elements, and is used to determine an element activation state profile for elements present on the surface of a cell.

In some embodiments, a chip comprises a multiplicity of the “second set binding elements,” in this case generally unlabeled. Such a chip is contacted with sample, preferably cell extract, and a second multiplicity of binding elements comprising element activation state specific binding elements is used in the sandwich assay to simultaneously determine the presence of a multiplicity of activated elements in sample. Preferably, each of the multiplicity of activation state-specific binding elements is uniquely labeled to facilitate detection.

In some embodiments, confocal microscopy can be used to detect activation profiles for individual cells. Confocal microscopy relies on the serial collection of light from spatially filtered individual specimen points, which is then electronically processed to render a magnified image of the specimen. The signal processing involved confocal microscopy has the additional capability of detecting labeled binding elements within single cells, accordingly in this embodiment the cells can be labeled with one or more binding elements. In some embodiments the binding elements used in connection with confocal microscopy are antibodies conjugated to fluorescent labels, however other binding elements, such as other proteins or nucleic acids are also possible.

In some embodiments, the methods and compositions of the instant invention can be used in conjunction with an “In-Cell Western Assay.” In such an assay, cells are initially grown in standard tissue culture flasks using standard tissue culture techniques. Once grown to optimum confluency, the growth media is removed and cells are washed and trypsinized The cells can then be counted and volumes sufficient to transfer the appropriate number of cells are aliquoted into microwell plates (e.g., Nunc™ 96 Microwell™ plates). The individual wells are then grown to optimum confluency in complete media whereupon the media is replaced with serum-free media. At this point controls are untouched, but experimental wells are incubated with a modulator, e.g. EGF. After incubation with the modulator cells are fixed and stained with labeled antibodies to the activation elements being investigated. Once the cells are labeled, the plates can be scanned using an imager such as the Odyssey Imager (LiCor, Lincoln Nebr.) using techniques described in the Odyssey Operator's Manual v1.2., which is hereby incorporated in its entirety. Data obtained by scanning of the multiwell plate can be analyzed and activation profiles determined as described below.

In some embodiments, the detecting is by high pressure liquid chromatography (HPLC), for example, reverse phase HPLC, and in a further aspect, the detecting is by mass spectrometry.

These instruments can fit in a sterile laminar flow or fume hood, or are enclosed, self-contained systems, for cell culture growth and transformation in multi-well plates or tubes and for hazardous operations. The living cells may be grown under controlled growth conditions, with controls for temperature, humidity, and gas for time series of the live cell assays. Automated transformation of cells and automated colony pickers may facilitate rapid screening of desired cells.

Flow cytometry or capillary electrophoresis formats can be used for individual capture of magnetic and other beads, particles, cells, and organisms.

Flexible hardware and software allow instrument adaptability for multiple applications. The software program modules allow creation, modification, and running of methods. The system diagnostic modules allow instrument alignment, correct connections, and motor operations. Customized tools, labware, and liquid, particle, cell and organism transfer patterns allow different applications to be performed. Databases allow method and parameter storage. Robotic and computer interfaces allow communication between instruments.

In some embodiment, the methods of the invention include the use of liquid handling components. The liquid handling systems can include robotic systems comprising any number of components. In addition, any or all of the steps outlined herein may be automated; thus, for example, the systems may be completely or partially automated. See U.S. Ser. No. 61/048,657.

As will be appreciated by those in the art, there are a wide variety of components which can be used, including, but not limited to, one or more robotic arms; plate handlers for the positioning of microplates; automated lid or cap handlers to remove and replace lids for wells on non-cross contamination plates; tip assemblies for sample distribution with disposable tips; washable tip assemblies for sample distribution; 96 well loading blocks; cooled reagent racks; microtiter plate pipette positions (optionally cooled); stacking towers for plates and tips; and computer systems.

Fully robotic or microfluidic systems include automated liquid-, particle-, cell- and organism-handling including high throughput pipetting to perform all steps of screening applications. This includes liquid, particle, cell, and organism manipulations such as aspiration, dispensing, mixing, diluting, washing, accurate volumetric transfers; retrieving, and discarding of pipet tips; and repetitive pipetting of identical volumes for multiple deliveries from a single sample aspiration. These manipulations are cross-contamination-free liquid, particle, cell, and organism transfers. This instrument performs automated replication of microplate samples to filters, membranes, and/or daughter plates, high-density transfers, full-plate serial dilutions, and high capacity operation.

In some embodiments, chemically derivatized particles, plates, cartridges, tubes, magnetic particles, or other solid phase matrix with specificity to the assay components are used. The binding surfaces of microplates, tubes or any solid phase matrices include non-polar surfaces, highly polar surfaces, modified dextran coating to promote covalent binding, antibody coating, affinity media to bind fusion proteins or peptides, surface-fixed proteins such as recombinant protein A or G, nucleotide resins or coatings, and other affinity matrix are useful in this invention.

In some embodiments, platforms for multi-well plates, multi-tubes, holders, cartridges, minitubes, deep-well plates, microfuge tubes, cryovials, square well plates, filters, chips, optic fibers, beads, and other solid-phase matrices or platform with various volumes are accommodated on an upgradable modular platform for additional capacity. This modular platform includes a variable speed orbital shaker, and multi-position work decks for source samples, sample and reagent dilution, assay plates, sample and reagent reservoirs, pipette tips, and an active wash station. In some embodiments, the methods of the invention include the use of a plate reader.

In some embodiments, thermocycler and thermoregulating systems are used for stabilizing the temperature of heat exchangers such as controlled blocks or platforms to provide accurate temperature control of incubating samples from 0° C. to 100° C.

In some embodiments, interchangeable pipet heads (single or multi-channel) with single or multiple magnetic probes, affinity probes, or pipetters robotically manipulate the liquid, particles, cells, and organisms. Multi-well or multi-tube magnetic separators or platforms manipulate liquid, particles, cells, and organisms in single or multiple sample formats.

In some embodiments, the instrumentation will include a detector, which can be a wide variety of different detectors, depending on the labels and assay. In some embodiments, useful detectors include a microscope(s) with multiple channels of fluorescence; plate readers to provide fluorescent, ultraviolet and visible spectrophotometric detection with single and dual wavelength endpoint and kinetics capability, fluorescence resonance energy transfer (FRET), luminescence, quenching, two-photon excitation, and intensity redistribution; CCD cameras to capture and transform data and images into quantifiable formats; and a computer workstation.

In some embodiments, the robotic apparatus includes a central processing unit which communicates with a memory and a set of input/output devices (e.g., keyboard, mouse, monitor, printer, etc.) through a bus. Again, as outlined below, this may be in addition to or in place of the CPU for the multiplexing devices of the invention. The general interaction between a central processing unit, a memory, input/output devices, and a bus is known in the art. Thus, a variety of different procedures, depending on the experiments to be run, are stored in the CPU memory.

These robotic fluid handling systems can utilize any number of different reagents, including buffers, reagents, samples, washes, assay components such as label probes, etc.

### Gating and Analysis

In some embodiments of the invention, different gating strategies can be used in order to analyze a specific cell population (e.g., only blasts) in a sample of mixed population after treatment with the modulator. These gating strategies can be based on the presence of one or more specific surface markers expressed on each cell type. In some embodiments, the first gate eliminates cell doublets so that the user can analyze singlets. The following gate can differentiate between dead cells and live cells and the subsequent gating of live cells classifies them into, e.g. myeloid blasts, monocytes and lymphocytes. A clear comparison can be carried out to study the effect of potential modulators, such as G-CSF on activable elements in: ungated samples, myeloid blasts, monocytes, granulocytes, lymphocytes, and/or other cell types by using two-dimensional contour plot representations, two-dimensional dot plot representations, and/or histograms. For example, a comparison can be carried out to study the effect of a modulator of the Jak/Stat signaling pathway in different cell populations within a patient sample by using two-dimensional contour plot representations of Stat5 and Stat3 phosphorylation (downstream intracellular readouts for Jak kinases) (X and Y axis). The level of basal phosphorylation and the change in phosphorylation in both Stat3 and Stat5 in response to a modulator such as G-CSF can be compared. G-CSF mediates increases in both Stat3 and Stat5 phosphorylation and this signaling can occur concurrently (subpopulations with increases in both p-Stat 3 and p-Stat5) or individually (subpopulations with either an increase in p-Stat3 or pStat5 alone). The advantage of gating is to get a clearer picture and more precise results of the effect of various activable elements on a specific cell sub-population such as blasts within a complex human sample.

In some embodiments, the present invention provides methods for classification, diagnosis, prognosis of a condition and/or prediction of outcome after administering a therapeutic agent to treat the condition by determining a drug transporter expression and/or function and/or by characterizing one or more pathways in a population of cells. The characterization of one or more pathways is performed by contacting a cell population with one or more modulators and determining the activation level of an activatable element of at least one cell in the cell population. The data can be analyzed using various metrics. Examples of metrics include: 1) measuring the difference in the log of the median fluorescence value between an unstimulated fluorochrome-antibody stained sample and a sample that has not been treated with a stimulant or stained (log(MFI_{Unstimulated Stained})−log(MFI_{Gated Unstained})), 2) measuring the difference in the log of the median fluorescence value between a stimulated fluorochrome-antibody stained sample and a sample that has not been treated with a stimulant or stained (log (MFI_{Stimulated Stained})−log(MFI_{Gated Unstained})), 3) Measuring the change between the stimulated fluorochrome-antibody stained sample and the unstimulated fluorochrome-antibody stained sample log(MFI_{Stimulated Stained})−log (MFI_{Unstimulated Stained}), also called “fold change in median fluorescence intensity”, 4) Measuring the percentage of cells in a Quadrant Gate of a contour plot which measures multiple populations in one or more dimension 5) measuring MFI of phosphor positive population to obtain percentage positivity above the background; and 6) use of multimodality and spread metrics for large sample population and for subpopulation analysis. Other possible metrics include third-color analysis (3D plots); percentage positive and relative expression of various markers; clinical analysis on an individual patient basis for various parameters, including, but not limited to age, race, cytogenetics, mutational status, blast percentage, CD34+ percentage, time of relapse, survival, etc. In alternative embodiments, there are other ways of analyzing data, such as third color analysis (3D plots), which can be similar to Cytobank 2D, plus third D in color In another embodiment, a user may analyze the signaling in subpopulations based on surface markers. For example, the user could look at: “stem cell populations” by CD34+ CD38− or CD34+ CD33− expressing cells; or drug transporter positive cells or cells identified based on their expression of the receptor for Flt3, or multiple leukemic subclones based on CD33, CD45, HLA-DR, CD11b and analyzing signaling in each subpopulation. In another alternative embodiment, a user may analyze the data based on intracellular markers, such as transcription factors or other intracellular proteins, based on a functional assay, or based on other fluorescent markers.

In some embodiments where flow cytometry is used, prior to analyzing of data the populations of interest and the method for characterizing these populations are determined. For instance, there are at least two general ways of identifying populations for data analysis: (i) “Outside-in” comparison of Parameter sets for individual samples or subset (e.g., patients in a trial). In this more common case, cell populations are homogenous or lineage gated in such a way as to create distinct sets considered to be homogenous for targets of interest. An example of sample-level comparison would be the identification of signaling profiles in tumor cells of a patient and correlation of these profiles with non-random distribution of clinical responses. This is considered an outside-in approach because the population of interest is pre-defined prior to the mapping and comparison of its profile to other populations. (ii) “Inside-out” comparison of Parameters at the level of individual cells in a heterogeneous population. An example of this would be the signal transduction state mapping of mixed hematopoietic cells under certain conditions and subsequent comparison of computationally identified cell clusters with lineage specific markers. This could be considered an inside-out approach to single cell studies as it does not presume the existence of specific populations prior to classification. A possible drawback of this approach is that it creates populations which, at least initially, may require multiple transient markers to enumerate and may never be accessible with a single cell surface epitope. As a result, the biological significance of such populations can be difficult to determine One advantage of this unconventional approach is the unbiased tracking of cell populations without drawing potentially arbitrary distinctions between lineages or cell types.

Each of these techniques capitalizes on the ability of flow cytometry to deliver large amounts of multiparameter data at the single cell level. For cells associated with a condition (e.g. neoplastic or hematopoetic condition), a third “meta-level” of data exists because cells associated with a condition (e.g. cancer cells) are generally treated as a single entity and classified according to historical techniques. These techniques have included organ or tissue of origin, degree of differentiation, proliferation index, metastatic spread, and genetic or metabolic data regarding the patient.

In some embodiments, the present invention uses variance mapping techniques for mapping condition signalling space. These methods represent a significant advance in the study of condition biology because it enables comparison of conditions independent of a putative normal control. Traditional differential state analysis methods (e.g., DNA microarrays, subtractive Northern blotting) generally rely on the comparison of cells associated with a condition from each patient sample with a normal control, generally adjacent and theoretically untransformed tissue. Alternatively, they rely on multiple clusterings and reclusterings to group and then further stratify patient samples according to phenotype. In contrast, variance mapping of condition states compares condition samples first with themselves and then against the parent condition population. As a result, activation states with the most diversity among conditions provide the core parameters in the differential state analysis. Given a pool of diverse conditions, this technique allows a researcher to identify the molecular events that underlie differential condition pathology (e.g., cancer responses to chemotherapy), as opposed to differences between conditions and a proposed normal control.

In some embodiments, when variance mapping is used to profile the signaling space of patient samples, conditions whose signaling response to modulators is similar are grouped together, regardless of tissue or cell type of origin. Similarly, two conditions (e.g. two tumors) that are thought to be relatively alike based on lineage markers or tissue of origin could have vastly different abilities to interpret environmental stimuli and would be profiled in two different groups.

When groups of signaling profiles have been identified it is frequently useful to determine whether other factors, such as clinical responses, presence of gene mutations, and protein expression levels, are non-randomly distributed within the groups. If experiments or literature suggest such a hypothesis in an arrayed flow cytometry experiment, it can be judged with simple statistical tests, such as the Student's t-test and the X² test. Similarly, if two variable factors within the experiment are thought to be related, the r² correlation coefficient from a linear regression is used to represent the degree of this relationship.

Examples of analysis for activatable elements are described in US publication number 20060073474 entitled “Methods and compositions for detecting the activation state of multiple proteins in single cells” and U.S. Pat. No. publication number 20050112700 entitled “Methods and compositions for risk stratification” the content of which are incorporate here by reference.

Advances in flow cytometry have enabled the individual cell enumeration of up to thirteen simultaneous parameters (De Rosa et al., 2001) and are moving towards the study of genomic and proteomic data subsets (Krutzik and Nolan, 2003; Perez and Nolan, 2002). Likewise, advances in other techniques (e.g. microarrays) allow for the identification of multiple activatable elements. As the number of parameters, epitopes, and samples have increased, the complexity of experiments and the challenges of data analysis have grown rapidly. An additional layer of data complexity has been added by the development of stimulation panels which enable the study of activatable elements under a growing set of experimental conditions. See Krutzik et al, Nature Chemical Biology February 2008. Methods for the analysis of multiple parameters are well known in the art. See U.S. Ser. No. 61/079,579 for gating analysis.

In some embodiments where flow cytometry is used, flow cytometry experiments are performed and the results are expressed as fold changes using graphical tools and analyses, including, but not limited to a heat map or a histogram to facilitate evaluation. One common way of comparing changes in a set of flow cytometry samples is to overlay histograms of one parameter on the same plot. Flow cytometry experiments ideally include a reference sample against which experimental samples are compared. Reference samples can include normal and/or cells associated with a condition (e.g. tumor cells). See also U.S. Ser. No. 61/079,537 for visualization tools.

The patients are stratified based on nodes that inform the clinical question using a variety of metrics. To stratify the patients between those patients with No Response (NR) versus a Complete Response (CR), a prioritization of the nodes can be made according to statistical significance (such as p-value or area under the curve) or their biological relevance.

### Methods

In some embodiments, the present invention provides methods for the classification, diagnosis, prognosis of a condition or prediction of outcome after administering a therapeutic agent to treat a condition; exemplary conditions include cancers such as AML, MDS and MPN. In other embodiments, the invention provides methods for monitoring and predicting the outcome of a condition after treatment with a therapeutic agent. In other embodiments, the invention provides methods for selection of a treatment for a condition. In other embodiments, the invention provide methods for drug screening to determine which drug or combination of drugs may be useful in a particular condition. In other embodiments, the invention provides methods for the identification of new druggable targets, that can be used alone or in combination with other treatments. In addition, the invention allows the selection of patients for specific target therapies.

In some embodiments, the present invention provides methods for classification, diagnosis, prognosis of a disease, and/or prediction of outcome after administering a therapeutic agent to treat the disease by characterizing a plurality of pathways in a population of cells. In some embodiments, the plurality of pathways is characterized by contacting a cell population with a therapeutic agent and determining the activation level of at least one activatable element within the cellular pathway being characterized. In some embodiments, the plurality of pathways is also characterized by contacting a cell population with one or more modulators and determining the activation level of at least one activatable element within the cellular pathway being characterized. The results from the pathways characterization are then correlated with the classification, diagnosis, prognosis of a disease, and/or prediction of outcome after administering a therapeutic agent to treat the disease. Examples of pathways that can be characterized with the methods described herein include DNA damage pathways, apoptosis pathways, cell cycle pathways, drug conversion into an active agent, internal cellular pH, redox potential environment, phosphorylation state of CD33 ITIM; drug activation; and signaling pathways for cytokines, chemokines and growth factors. In some embodiments, the methods further comprises determining drug binding. In some embodiments, the therapeutic agent is an agent to treat cancer. In some embodiments, the therapeutic agent is a DNA damage and/or apoptosis inducing agent. In some embodiments, the pathways being characterized are DNA damage and apoptosis pathways. In some embodiments, the therapeutic agent is a DNA damage and/or apoptosis inducing agent. In some embodiments, the therapeutic agent is Mylotarg.

Thus, in some embodiments, the invention provides methods for classification, diagnosis, prognosis of a disease, and/or prediction of outcome after administering a therapeutic agent to treat the disease by contacting a cell population with a therapeutic agent and determining the activation level of at least one activatable element within the DNA damage and apoptosis pathways. In some embodiments, the activatable element within the DNA damage pathway is selected from the group consisting of p-53, p-Chk1, p-Chk2, and p-ATM. In some embodiments, the activatable element within the apoptosis pathway is selected from the group consisting of cleaved PARP and cleaved Caspase 3. Other pathways can be further characterized simultaneously with the DNA damage and apoptosis pathways by contacting the cell population with a modulator and determining the activation level of at least one activatable element within pathways being characterized. Example of such pathways include cellular redox, phosphorylation state of CD33 ITIM, intracellular pH, drug conversion into an active agent, and signaling pathways for cytokines, chemokines and growth factors. The results from the characterization of the DNA damage and apoptosis pathways together with the characterization of one or more pathways described herein are then correlated with the classification, diagnosis, prognosis of a disease, and/or prediction of outcome after administering a therapeutic agent to treat the disease. In some embodiments, the methods further comprises determining drug binding In some embodiments, the therapeutic agent is a DNA damage and/or apoptosis inducing agent. In some embodiments, the therapeutic agent is Mylotarg.

In some embodiments, the Jak/Stat, PI3K/Akt, MAPK or cell cycle pathways are characterized simultaneously with the DNA damage and apoptosis pathways by contacting the cell population with one or more modulators and determining the activation level of at least one activatable element within the pathways. In some embodiments, the activatable element within the PI3K/AKT or MAPK pathways is selected from the group consisting of p-Akt, p-ERK, p38 and pS6 and the modulator is selected from the group consisting of FLT3L, SCF, G-CSF, SCF, G-CSF, SDF1a, LPS, PMA, and Thapsigargin. In some embodiments, the activatable element within the PI3K/AKT or MAPK pathways is selected from the group consisting of p-Akt, p-ERK, and pS6 and the modulator is selected from the group consisting of SCF, and PMA. In some embodiments, the activatable element within the STAT pathway is selected from the group consisting of p-Stat3, p-Stat5, p-Stat1, and p-Stat6 and the modulator is selected from the group consisting of IFNg, IFNa, IL-27, IL-3, IL-6, IL-10, and G-CSF. In some embodiments, the activatable element within the STAT pathway is p-Stat1 and the modulator is IL-6. In some embodiments, the activatable element within a cell cycle pathway is selected from the group consisting of p-Cdc25, p-p53, p-CyclinA-Cdk2, p-CyclinE-Cdk2, p-CyclinB-Cdk1, p-p21, p-Histone H3 and p-Gadd45, and the modulator is selected from the group consisting of Staurosporine, Etoposide, Mylotarg, Daunorubicin, and AraC. In some embodiments, the activatable element within a cell cycle pathway is selected from the group consisting of p-p53, p-CyclinB-Cdk, and p-Histone H3, and the modulator is Mylotarg.

In some embodiments, the population of cells is a population of hematopoietic cells. Examples of hematopoietic cells include, but are not limited to pluripotent hematopoietic stem cells, T-lymphocyte lineage progenitor or derived cells, B-lymphocyte lineage progenitor or derived cells, granulocyte lineage progenitor or derived cells, monocyte lineage progenitor or derived cells, megakaryocyte lineage progenitor or derived cells and erythroid lineage progenitor or derived cells.

In some embodiments, the present invention provides methods for classification, diagnosis, prognosis of a disease and/or prediction of outcome after administering a therapeutic agent to treat the disease by determining a drug transporter expression and/or function. Methods for determining drug transporter expression and/or function are known in the art, including those described herein. Examples of drug transporters include, but are not limited to, P-glycoprotein (Pgp/MDR1/ABCB1), MDR-associated protein (MRP1, ABCC1) and breast cancer resistance protein (BCRP, ABCG2 or MXR). In some embodiments the drug transporter is MDR1. The results from the drug transporter expression and/or function assay is then correlated to the classification, diagnosis, prognosis of a disease and/or prediction of outcome after administering a therapeutic agent to treat the disease. In some embodiments, the therapeutic agent is a DNA damage and/or apoptosis inducing agent. In some embodiments, the therapeutic agent is a drug transporter substrate. In some embodiments, the therapeutic agent is Mylotarg. In some embodiments, the population of cells is a population of hematopoietic cells.

In some embodiments, the present invention provides methods for classification, diagnosis, prognosis of disease and/or prediction of outcome after administering a therapeutic agent to treat the disease by determining a drug transporter expression and/or function and by characterizing one or more pathways in a population of cells. In some embodiments, the methods comprise the step of: (i) contacting a cell population with a therapeutic agent; (ii) determining a drug transporter expression and/or function; and (iii) determining the activation level of at least one activatable element within the cellular pathway being characterized. In some embodiments, the plurality of pathways is also characterized by contacting the cell population with one or more modulators and determining the activation level of at least one activatable element within the cellular pathway being characterized. The results from the characterization of the pathways and drug transporter expression and/or function determination are then correlated with the classification, diagnosis, prognosis of a disease, and/or prediction of outcome after administering a therapeutic agent to treat the disease. Examples of pathways that can be characterized with the methods described herein include DNA damage pathways, apoptosis pathways, cell cycle pathways, drug conversion into an active agent, internal cellular pH, redox potential environment, phosphorylation state of CD33 ITIM; drug activation; and signaling pathways in response to cytokines, chemokines and/or growth factors. In some embodiments, the methods further comprises determining drug binding. In some embodiments, the therapeutic agent is an agent to treat cancer. In some embodiments, the therapeutic agent is a DNA damage and/or apoptosis inducing agent. In some embodiments, the pathways being characterized are DNA damage and apoptosis pathways. In some embodiments, the therapeutic agent is a therapeutic to treat cancer. In some embodiments, the therapeutic agent is a DNA damage and/or apoptosis inducing agent. In some embodiments, the therapeutic agent is a drug transporter substrate. In some embodiments, the therapeutic agent is Mylotarg. In some embodiments the drug transporter is MDR1. In some embodiments, the population of cells is a population of hematopoietic cells.

Thus, in some embodiments, the invention provides methods for classification, diagnosis, prognosis of a disease, and/or prediction of outcome after administering a therapeutic agent to treat the disease by contacting a cell population with a therapeutic agent, determining a drug transporter expression and/or function and determining the activation level of at least one activatable element within the DNA damage and apoptosis pathways. In some embodiments, the activatable element within the DNA damage pathway is selected from the group consisting of p-p53, p-Chk1, p-Chk2, and p-ATM. In some embodiments, the activatable element within the apoptosis pathway is selected from the group consisting of cleaved PARP and cleaved Caspase 3. Other pathways can be further characterized simultaneously with the DNA damage and apoptosis pathways and the drug transporter expression and/or function by contacting the cell population with a modulator and determining the activation level of at least one activatable element within pathways being characterized. Examples of such pathways include cellular redox, the phosphorylation state of CD33 ITIM, intracellular pH, drug conversion into an active agent, and signaling pathways for cytokines, chemokines and growth factors. In some embodiments, the methods further comprises determining drug binding. The results from the DNA damage and apoptosis pathways characterization and the drug transporter expression and/or function determination together with the characterization of one or more additional pathways described herein are then correlated with the classification, diagnosis, prognosis of a disease, and/or prediction of outcome after administering a therapeutic agent to treat the disease. In some embodiments, the therapeutic agent is a DNA damage and/or apoptosis inducing agent. In some embodiments, the therapeutic agent is a drug transporter substrate. In some embodiments, the therapeutic agent is Mylotarg. In some embodiments, the drug transporter is MDR1. In some embodiments, the population of cells is a population of hematopoietic cells.

In some embodiments, the Jak/Stat, PI3K/Akt, MAPK or cell cycle pathways are characterized simultaneously with the DNA damage and apoptosis pathways, and the drug transporter expression and/or function determination by contacting the cell population with one or more modulator and determining the activation level of at least one activatable element within the pathways. In some embodiments, the activatable element within the PI3K/AKT or MAPK pathways is selected from the group consisting of p-Akt, p-ERK, p38 and pS6 and the modulator is selected from the group consisting of FLT3L, SCF, G-CSF, SCF, G-CSF, SDF1a, LPS, PMA, and Thapsigargin. In some embodiments, the activatable element within the PI3K/AKT or MAPK pathways is selected from the group consisting of p-Akt, p-ERK, and pS6 and the modulator is selected from the group consisting of SCF, and PMA. In some embodiments, the activatable element within the STAT pathway is selected from the group consisting of p-Stat3, p-Stat5, p-Stat1, and p-Stat6 and the modulator is selected from the group consisting of IFNg, IFNa, IL-27, IL-3, IL-6, IL-10, and G-CSF. In some embodiments, the activatable element within the STAT pathway is p-Stat1 and the modulator is IL-6. In some embodiments, the activatable element within a cell cycle pathway is selected from the group consisting of Cdc25, p53, CyclinA-Cdk2, CyclinE-Cdk2, CyclinB-Cdk1, p21, p-Histone H3 and Gadd45, and the modulator is selected from the group consisting of Staurosporine, Etoposide, Mylotarg, Chlofarabine, Daunorubicin, and AraC. In some embodiments, the activatable element within a cell cycle pathway is selected from the group consisting of p53, CyclinB-Cdk, and p-Histone H3, and the modulator is Mylotarg. In some embodiments, the population of cells is a population of hematopoietic cells.

In some embodiments, a treatment or a combination of treatments is chosen based on the characterization of plurality of pathways in single cells and the function and/or expression of a drug transporter. In some embodiments, characterizing a plurality of pathways in single cells comprises determining whether apoptosis pathways, cell cycle pathways, or DNA damage pathways are functional in an individual in response to a therapeutic agent based on the activation levels of activatable elements within the pathways, where a pathway is functional if the activatable elements within the pathways change their activation state in response to the therapeutic agent. For example, when the apoptosis, cell cycle, signaling, and DNA damage pathways are functional the individual may be able to respond to treatment, and when at least one of the pathways is not functional the individual may not be able to respond to treatment. In some embodiments, if the apoptosis and DNA damage pathways are functional the individual can respond to treatment. In some embodiments, the population of cells is a population of hematopoietic cells.

In some embodiments, an increase in the activation levels of an activatable element within the PI3K/Akt and/or MAPK pathway in a cell population in response to a modulator is indicative that the cell population is resistant to treatment with a therapeutic agent. In some embodiments, the activatable element within the PI3K/AKT or MAPK pathways is selected from the group consisting of p-Akt, p-Erk, and pS6 and the modulator is selected from the group consisting of SCF, and PMA. In some embodiments, an increased in the activation levels of an activatable element within the PI3K/Akt and/or MAPK pathway in a cell population in response to a modulator is indicative that the cell population could be sensitized to the therapeutic agent by contacting the cell population with a PI3K and/or Mek inhibitors. PI3K and/or Mek inhibitors are known in the art. In some embodiments, the population of cells is a population of hematopoietic cells.

In some embodiments, an increase in the activation levels of an activatable element within the Jak/Stat in a cell population in response to a modulator is indicative that the cell population is sensitive to treatment with a therapeutic agent. In some embodiments, the activatable element within the STAT pathway is selected from the group consisting of p-Stat3, p-Stat5, p-Stat1, and p-Stat6 and the modulator is selected from the group consisting of IFNg, IFNa, IL-27, IL-3, IL-6, IL-10, and G-CSF. In some embodiments, the activatable element within the STAT pathway is p-Stat1 and the modulator is IL-6. In some embodiments, the population of cells is a population of hematopoietic cells.

In some embodiments, an increased in the activation levels of an activatable element within the cell cycle in a cell population in response to a modulator is indicative that the cell population has undergone cell cycle arrest and is sensitive to treatment with a therapeutic agent. In some embodiments, no increased in the activation levels of an activatable element within the cell cycle in a cell population in response to a modulator is indicative that the cell population has not undergone cell cycle arrest and the population is resistant to treatment with a therapeutic agent. In some embodiments, the activatable element within a cell cycle pathway is selected from the group consisting of p-Cdc25, p-p53, p-CyclinA-Cdk2, p-CyclinE-Cdk2, p-CyclinB-Cdk1, p-p21, p-Histone H3 and p-Gadd45, and the modulator is selected from the group consisting of Staurosporine, Etoposide, Mylotarg, Daunorubicin, Chlofarabine and AraC. In some embodiments, the activatable element within a cell cycle pathway is selected from the group consisting of p-p53, p-CyclinB-Cdk, and p-Histone H3, and the modulator is Mylotarg. In some embodiments, the population of cells is a population of hematopoietic cells.

In some embodiments, the invention provides methods for the delineation of subpopulations of cells associated with a condition that are differentially susceptible to a therapeutic agent or therapeutic agent combinations. In another embodiment, the invention provides methods to demarkate subpopulations of cells associated with a condition that have different genetic subclone origins. In another embodiment, the invention provides for the identification of a cell type, that in combination with other cell type(s), provide ratiometric or metrics that singly or coordinately allow for surrogate identification of subpopulations of cells associated with a disease, diagnosis, prognosis, disease stage of the individual from which the cells were derived, response to treatment, monitoring and predicting outcome of disease.

One aspect of the invention involves contacting a hematopoietic cell with a therapeutic agent designed to treat cancer cells, wherein resistant cells may arise during a therapeutic treatment using the therapeutic agent; contacting the cells with the therapeutic agent; analyzing the activation levels of activatable elements within the following pathways by flow cytometry in which individual cells are simultaneously analyzed for multiple characteristics: cellular redox, phosphorylation state of CD33 ITIM, intracellular pH, drug transporter function; drug transporter expression; drug conversion into an active agent; signaling pathways for cytokines, growth factors, DNA damage repair, and apoptosis; and correlating the results of the analysis with response to the compound as a function of each of the modulators; determining the activation states of a plurality of activatable elements in the cell; and classifying the cell based on said activation state. In one embodiment, the compound is a conjugate between a binding agent, such as an antibody or similar binding entity and a cytotoxic or apoptotic agent. In some embodiments, the methods further comprises determining drug binding.

In some embodiments, this invention is directed to methods and compositions, and kits for analysis, drug screening, diagnosis, prognosis, for methods of disease treatment and prediction. In some preferred embodiments, a therapeutic agent is contacted with cells to analyze the response to the compound. Responses may include primary refractory behavior (resistance), positive response (full or partial), and other indications such as intensity or duration of response including time of relapse. The results may be useful to determine treatment, understand whether a treatment will work, monitor treatment, modify therapeutic regimens, and to further optimize the selection of therapeutic agents which may be administered as one or a combination of agents. Hence, therapeutic regimens can be individualized and tailored according to the data obtained prior to, and at different times over the course of treatment, thereby providing a regimen that is individually appropriate.

The methods of the invention provide tools useful in the treatment of an individual afflicted with a condition, including but not limited to methods for assigning a risk group, methods of predicting an increased risk of relapse, methods of predicting an increased risk of developing secondary complications, methods of choosing a therapy for an individual, methods of predicting duration of response, response to a therapy for an individual, methods of determining the efficacy of a therapy in an individual, and methods of determining the prognosis for an individual. The present invention provides methods that can serve as a prognostic indicator to predict the course of a condition, e.g. whether the course of a neoplastic or a hematopoietic condition in an individual will be aggressive or indolent, thereby aiding the clinician in managing the patient and evaluating the modality of treatment to be used. In another embodiment, the present invention provides information to a physician to aid in the clinical management of a patient so that the information may be translated into action, including treatment, prognosis or prediction.

In some embodiments, the invention is directed to methods for determining the activation level of one or more activatable elements in a cell upon treatment with one or more modulators. The activation of an activatable element in the cell upon treatment with one or more modulators can reveal operative pathways in a condition that can then be used, e.g., as an indicator to predict course of the condition, to identify risk group, to predict an increased risk of developing secondary complications, to choose a therapy or combination therapy for an individual, to predict response to a therapy for an individual, to determine the efficacy of a therapy in an individual, and to determine the prognosis for an individual.

In some embodiments, the invention is directed to methods for classifying a cell by contacting the cell with a modulator, including for example a proposed therapeutic, determining the presence or absence of an increase in activation level of an activatable element in the cell, and classifying the cell based on the presence or absence of the increase in the activation of the activatable element. In some embodiments, the invention is directed to methods of determining the presence or absence of a condition in an individual by subjecting a cell from the individual to a modulator and an inhibitor, determining the activation level of an activatable element in the cell, and determining the presence or absence of the condition based on the activation level upon treatment with a modulator and an inhibitor.

In some embodiments, the invention is directed to methods of determining a phenotypic profile (or signature) of a population of cells by exposing the population of cells to a modulator or a plurality of modulators in separate cultures, wherein at least one of the modulators is an inhibitor, determining the presence or absence of an increase in activation level of an activatable element in the cell population from each of the separate culture and classifying the cell population based on the presence or absence of the increase in the activation of the activatable element from each of the separate culture.

In some embodiments, expression markers are analyzed in addition to activatable elements. The expression markers may be detected using many different techniques, for example using nodes from flow cytometry data (see the articles and patent applications referred to above). Other common techniques employ expression arrays (commercially available from Affymetrix, Santa Clara Calif.), taqman (commercially available from ABI, Foster City Calif.), SAGE (commercially available from Genzyme, Cambridge Mass.), sequencing techniques (see the commercial products from Helicos, 454, US Genomics, and ABI) and other commonly know assays. See Golub et al., Science 286: 531-537 (1999). Expression markers are measured in unstimulated cells to know whether the stimulation has an impact on functional apoptosis. This provides implications for treatment and prognosis for the disease. Under this hypothesis, the amount of drug transporters correlates with the response of the patient and non-responders may have more levels of drug transporters (to move a drug out of a cell) as compared to responders. In some embodiments, the invention is directed to methods of classifying a cell population by contacting the cell population with at least one modulator that affects signaling mediated by receptors or determining the activation states of a plurality of activatable elements in the cell; and classifying the cell based on said activation states and expression levels.

The term “patient” or “individual” as used herein includes humans as well as other mammals. The methods generally involve determining the status of an activatable element. The methods also involve determining the status of a plurality of activatable elements.

The classification of a cell according to the status of an activatable element can comprise classifying the cell as a cell that is correlated with a clinical outcome. In some embodiments, the clinical outcome is the prognosis and/or diagnosis of a condition. In some embodiments, the clinical outcome is the presence or absence of a neoplastic or a hematopoietic condition. Example conditions are acute myeloid leukemia (AML), myelodysplastic syndrome (MDS) or myeloproliferative disorders (MPDS) also known as myeloproliferative disorders (MPNs). See U.S. Application 61/085,789 which is incorporated by reference. In some embodiments, the clinical outcome is the staging or grading of a neoplastic or hematopoietic condition. Examples of staging include, but are not limited to, refractory, WHO classification, FAB classification, IPSS score, WPSS score, extensive stage, staging according to cellular markers, occult, including information that may inform on time to progression, progression free survival, overall survival, or event-free survival.

The analysis of a cell and the determination of the status of an activatable element can lead to classifying a cell as a cell that is correlated to a patient response to a treatment. In some embodiments, the patient response is selected from the group consisting of complete response, partial response, no response, resistance/refractory, progressive disease, stable disease and adverse reaction. Duration of response may be determined in some embodiments.

In some embodiments, the invention provides methods for the classification of rare cells. The classification of a rare cell according to the activation level of an activatable element can comprise classifying the cell as a cell that can be correlated with minimal residual disease or emerging resistance. See U.S. No. 61/048,886 which is incorporated by reference.

The classification of a cell according to the activation level of an activatable element can comprise selecting a method of treatment. Example of methods of treatments include, but are not limited to chemotherapy, biological therapy, radiation therapy, bone marrow transplantation, Peripheral stem cell transplantation, umbilical cord blood transplantation, autologous stem cell transplantation, allogeneic stem cell transplantation, syngeneic stem cell transplantation, surgery, induction therapy, maintenance therapy, salvage therapy, and other therapy. Example therapies may include aspirin, human growth factors such as EPO and G-CSF, etc.

In some embodiments, cells (e.g. normal cells) other than the cells associated with a condition (e.g. cancer cells) or a combination of cells are used, e.g., in assigning a risk group, predicting an increased risk of relapse, predicting an increased risk of developing secondary complications, choosing a therapy for an individual, predicting response to a therapy for an individual, determining the efficacy of a therapy in an individual, and/or determining the prognosis for an individual. For instance, in the case of cancer, infiltrating immune cells might determine the outcome of the disease. Alternatively, a combination of information from the cancer cell plus the immune cells in the blood that are responding to the disease, or reacting to the disease can be used for diagnosis or prognosis of the cancer.

In some embodiments, the invention provides methods to carry out multiparameter flow cytometry to monitor phospho-protein responses to various compounds designed to treat cancer and factors in acute myeloid leukemia MDS, or MPDS (as example diseases) at the single cell level. Phospho-protein members of signaling cascades and the kinases and phosphatases that interact with them are required to initiate and regulate proliferative signals in cells. Apart from the basal level of protein phosphorylation alone, the effect of potential drug molecules on these network pathways was studied to discern unique cancer network profiles, which correlate with the genetics and disease outcome. Single cell measurements of phospho-protein responses reveal shifts in the signaling potential of a phospho-protein network, enabling categorization of cell network phenotypes by multidimensional molecular profiles of signaling. See U.S. Pat. No. 7,393,656. See also Irish et. al., Single cell profiling of potentiated phospho-protein networks in cancer cells. Cell. 2004, vol. 118, p. 1-20.

Flow cytometry is useful in a clinical setting, since relatively small sample sizes, as few as 10,000 cells, can produce a considerable amount of statistically tractable multidimensional signaling data and reveal key cell subsets that are responsible for a phenotype. See U.S. Pat. Nos. 7,381,535 and 7,393,656. See also Krutzik et al, 2004.

Cytokine response panels have been studied to survey altered signal transduction of cancer cells by using a multidimensional flow cytometry file which contained at least 30,000 cell events. In one embodiment, this panel is expanded and the effect of growth factors and cytokines on primary AML samples studied. See U.S. Pat. Nos. 7,381,535 and 7,393,656. See also Irish et. al., Cell. vol. 118, p. 1-20 (2004).

In some embodiments, the analysis involves working at multiple characteristics of the cell in parallel after contact with a therapeutic agent. For example, the analysis can examine drug transporter function; drug transporter expression; drug conversion into an active agent; cellular redox potential; signaling pathways; DNA damage repair; and apoptosis. Analysis can assess the ability of the cell to undergo the process of apoptosis after exposure to the experimental drug in an in vitro assay as well as how quickly the drug is exported out of the cell or metabolized. The drug response panel can include but is not limited to detection of phosphorylated Chk2, and phosphorylated H2AX, cleaved Caspase 3, cleaved Caspase 8, and cleaved PARP, and determinations of mitochondrial cytochrome C. Modulators may include Mylotarg, Staurosprine, Etoposide, chlofarabine, AraC, and daunorubicin. Analysis can assess phosphatase activity after exposure of cells to phosphatase inhibitors including but not limited to 3 mM hydrogen peroxide (H₂O₂), alone or in combination with other modulators (3 mM H₂O₂+SCF and 3 mM H₂O₂+IFNα) or other phosphatase inhibitors. The response panel to evaluate phosphatase activity can include but is not limited to the detection of phosphorylated Slp76, PLCg2, Lck, S6, Akt, Erk, Stat1, Stat3, and Stat5. Later, the samples may be analyzed for the expression of drug transporters such as MDR1/PGP, MRP1 and BCRP/ABCG2. Samples may also be examined for XIAP, Survivin, Bcl-2, MCL-1, Bim, Ki-67, Cyclin D1, Id1, Bcl-X_{L}, Pim1, Pim2, and Myc.

### Drug Response Signatures

In some embodiments, the invention may identify a set of drug response signatures in disease cells. The drug response signature is a profile of the activation levels of activatable elements following in vitro exposure of patient samples to a drug and one or more modulators, and can predict whether patients may respond to the drug. If the cells do not respond to the drug, the activation states of specific pathways may indicate the biological basis for the lack of response, the methods of the invention may be used to select combination therapies, and screen these combination therapies treating cells with the original drug in conjunction with the combination therapy. As an example, the methods of the invention may be used to identify signatures in AML cells taken from patients based on DNA damage response, as measured by p-H2AX levels and p-CHK2 levels, and induction of apoptosis, as measured by cleaved PARP, amine aqua staining and forward and side scatter of light (see FIGS. 2-4). In some embodiments, other measurements of cell viability may be used, for example cleaved caspase 3, or amount of dyes staining or other stains, for example: propidium idodide, SYTOX, TUNEL, or acridine orange. In some embodiments, other measurements of DNA Damage may be used, for example p-Chk1, p-ATM or p-p53. p-H2AX is a phosphorylated histone H2 variant that nucleates a DNA damage response complex. For review, see van Attikum H., and Gasser S. M. Trends in Cell Biology 19: pp. 207-17 (2009). ATR and ATM kinase activity in response to DNA damage activates Chk1 and Chk2, respectively. P-Chk1 and p-Chk2 amplify the DNA damage response by inducing cell cycle arrest and apoptosis. For review, see Abraham, R. T. Cell cycle checkpoint signaling through the ATM and ATR kinases. Genes & Dev. 15: 2177-96 (2001). Healthy myeloid cells respond to Mylotarg treatment with increased levels of p-Chk2, p-H2AX, p-ATM, p-P53 and increases in cleaved PARP and apoptosis at 72 h (See U.S. Provisional Application No. 61/186,619). This is also seen at earlier timepoints. Myeloid cells from AML patients exhibit three types of response signatures to Mylotarg treatment:

Signature 1 is a complete in vitro response, comprising (a) Mylotarg-induced DNA damage response, indicated for example, by high levels of p-Chk2 and p-H2AX, and (b) Mylotarg-induced apoptosis, indicated, for example, by high levels of cleaved PARP, cleaved caspase 3, amine aqua staining, and forward and side scatter of light indicating cell death (See U.S. Provisional Application No. 61/186,619). If patient cell samples exhibit Signature 1 in vitro, these patients may respond to Mylotarg treatment. The methods of the invention can be used to determine Mylotarg dosing. The methods of the invention may also be used to screen for additional therapeutics that enhance the efficacy of Mylotarg or reduce off-target effects in these patients.

Signature 2 is an incomplete in vitro response, comprising (a) Mylotarg-induced DNA damage response, indicated, for example, by high levels of p-Chk2 and p-H2AX, but (b) no Mylotarg-induced apoptosis, indicated, for example, by baseline levels of cleaved PARP and cleaved Caspase 3, forward and side scatter of light, and amine aqua staining (See U.S. Provisional Application No. 61/186,619). When a patient's myeloid cells exhibit Signature 2 in vitro, the DNA damage response demonstrates that these cells are responding to Mylotarg, and Mylotarg is being internalized and activated inside of these cells. However, the lack of apoptosis suggests that there may be a defect that disconnects the DNA damage response pathway from activation of apoptosis. Thus, these patients may not respond to Mylotarg alone, but may respond to a combination of therapeutics that includes Mylotarg. The methods of the invention can be used to screen patient cells for Mylotarg in combination with one or more additional therapies, or for combinations of one or more additional therapeutics without Mylotarg to identify a therapy to which the patient cells are likely to respond. Other therapeutics may be selected, including, but not limited to the following: CSL-360, regrafomib, obatoclax, GDC-0152, GBL-310, ABT-263, phenoxodiol, SGI-1776, AT-101, ABT-869, NRX-5183, AC-220, AS-1411, ARRY-520, AZD-1152, AZD-4877, cediranib (Recentin), L-Vax, Sorafenib, BI-2536, BI-6727, BI-811283, cytarabine, daunorubicin, bortezomib, alitretinoin, LOR-2040, annamycin, PR1 peptide antigen vaccine, vorinostat, MG-98, mocetinostat dihydrobromide, ubenimex, elacytarabine, imatinib (Gleevec), midostaurin, valspodar (cyclosporin A derivative), vatalanib (finasunate), lintuzumab, axitinib, decitabine, SCH-727965, plitidepsin (Aplidine), arsenic trioxide (Trisenox), fostamatinib (tamatinib fosdium), tretinoin, sapacitabine, cladribine, clofarabine (Clolar; Clofarex; Evoltra), sunitinib (Sutent), oncohist, temozolomide (Temodar; Temodal), tamibarotene (Tamibaro; Amnoid; Amnolake), belinostat, azacitidine (Vidaza), DT3881L-3, amonafide malate (Xanafide), Volorexin, or other apoptosis promoting agents.

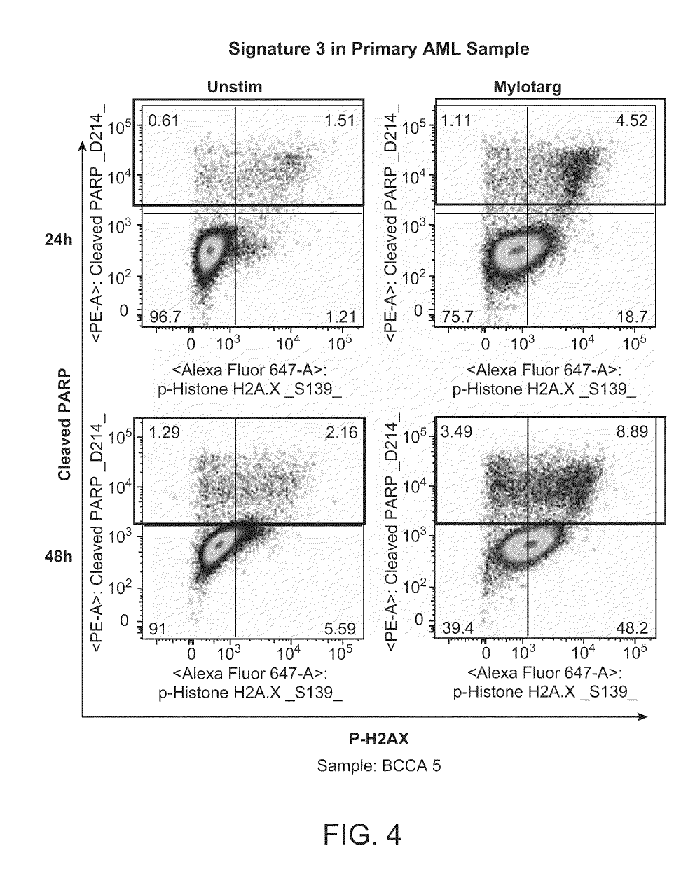

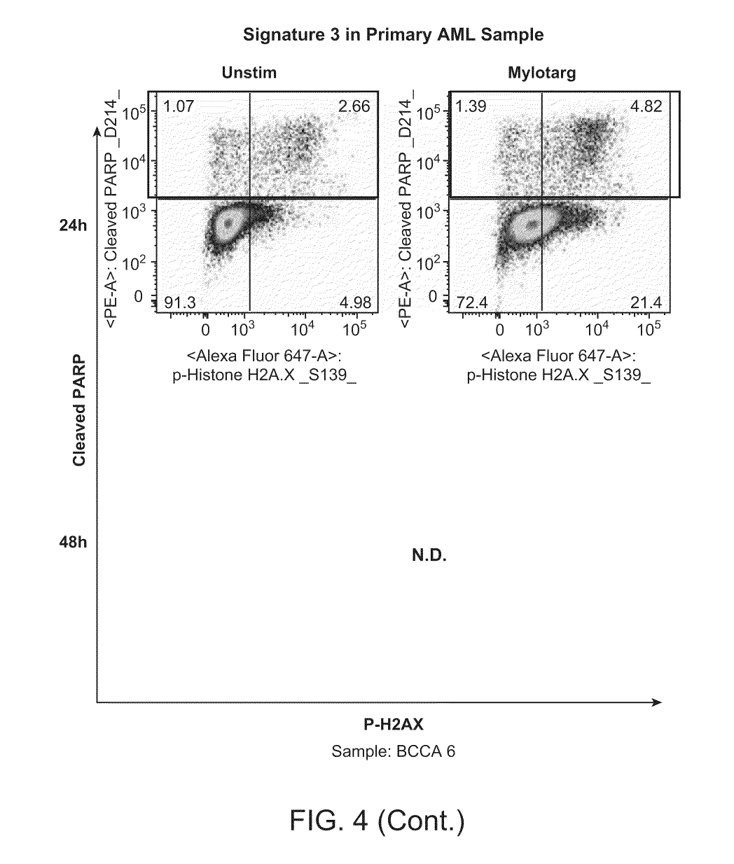

Signature 3 is a non-response in vitro, comprising (a) no Mylotarg-induced DNA damage response, indicated, for example, by baseline levels of p-Chk2 and p-H2AX, and (b) no Mylotarg-induced apoptosis, indicated, for example, by baseline levels of cleaved Caspase 3 and cleaved PARP, forward and side scatter of light, and amine aqua staining (See FIG. 4 and See U.S. Provisional Application No. 61/186,619). When a patient's myeloid cells exhibit Signature 3, the lack of DNA damage or apotosis suggests these cells do not respond to Mylotarg alone, perhaps due to insufficient binding, internalization, or processing of Mylotarg. One possibility is that these cells display low levels of CD33, making the patient a poor candidate for treatment with and anti-CD33 antibody such as Mylotarg. To evaluate this possibility, the methods of the invention can be used to assess CD33 levels on patient's cells. Another possibility is that drug transporter activity extrudes drug out of the cell, suggesting that a Mylotarg response may be induced if patients are treated with a combination of Mylotarg and a drug transporter inhibitor. The methods of the invention can be used to identify combinations of therapeutics that enhance Mylotarg response. For example, treating cell lines with a combination of Mylotarg and the drug transporter inhibitor cyclosporine A increases p-H2AX levels, p-ATM, p-p53 and p-Chk2 levels (See U.S. Provisional Application No. 61/186,619). One skilled in the art will appreciate that the methods of the invention may be used for inhibitors or combinations of inhibitors of other drug transporters, including, but not limited to PSC-833 for MDR1, reserpine for BCRP-1 and MK571 or probenecid for MRP-1. Another possibility is that Mylotarg is internalized but not processed. The methods of the invention can be used to assay Mylotarg internalization, for example, by fixing patient cells and immunofluorescently staining for Mylotarg, and then permeablizing these cells and immunofluorescently staining for Mylotarg using a different fluorophore. Comparing the levels of each to the two fluorophores, for example by flow cytometry, can identify the respective amount of external and internalized Mylotarg in individual cells. Furthermore, the methods of the invention can be used to profile metabolic pathways involved in processing Mylotarg in patient cells. For example, fluorescent dyes may be used assess pH or reductive capacity within lysozomes, which affect cleavage of the linker in Mylotarg. Flow cytometry can then be used to profile cells or populations of cells for Mylotarg internalization and metabolic pathway function to determine whether inadequate internalization or processing is interfering with Mylotarg activity, and to identify classes of compounds that may restore Mylotarg activity. The methods of the invention may then be used to identify combinations of Mylotarg with other compounds that may increase the efficacy of Mylotarg in patients. In some instances, AML patients will contain subpopulation of cells with two signatures or with all three signatures. The methods of the invention may then be used to identify combinations of Mylotarg with other compounds that may increase the efficacy of Mylotarg in patients in the different subpopulations or identify compounds or combination of compounds to treat the different subpopulations.

In another example, peripheral blood mononuclear cells and bone marrow mononuclear cells derived from AML patients exhibit different expression marker signatures in response to Mylotarg treatment. Peripheral blood mononuclear cells and bone marrow mononuclear cells derived from AML and healthy samples were exposed to mylotarg for 6 hours. In that time a reduction was seen in the expression levels of the CD33 and CD13 phenotypic markers for myeloid cells derived from both sample sources. In some embodiments, changes in CD33 and CD 13 levels following Mylotarg treatment may be used as an internal control to verify that cells were in fact treated with functional Mylotarg, regardless of the cells' specific response signature or signatures. CD13 levels are also reduced following twenty-four (24) hours of exposure to Mylotarg. The reduction in CD13 expression levels is specific to that marker since CD11b (also a myeloid marker) levels remain unchanged following the same exposure conditions to mylotarg.

In other embodiments of the invention, drug response signatures may be identified for drugs other than Mylotarg, including, but not limited to: chlofarabine, CSL-360, regrafomib, obatoclax, GDC-0152, GBL-310, ABT-263, phenoxodiol, SGI-1776, AT-101, ABT-869, NRX-5183, AC-220, AS-1411, ARRY-520, AZD-1152, AZD-4877, cediranib (Recentin), L-Vax, Sorafenib, BI-2536, BI-6727, BI-811283, cytarabine, daunorubicin, bortezomib, alitretinoin, LOR-2040, annamycin, PR1 peptide antigen vaccine, vorinostat, MG-98, mocetinostat dihydrobromide, ubenimex, elacytarabine, imatinib (Gleevec), midostaurin, valspodar (cyclosporin A derivative), vatalanib (finasunate), lintuzumab, axitinib, decitabine, SCH-727965, plitidepsin (Aplidine), arsenic trioxide (Trisenox), fostamatinib (tamatinib fosdium), tretinoin, sapacitabine, cladribine, clofarabine (Clolar; Clofarex; Evoltra), sunitinib (Sutent), oncohist, temozolomide (Temodar; Temodal), tamibarotene (Tamibaro; Amnoid; Amnolake), belinostat, azacitidine (Vidaza), DT3881L-3, amonafide malate (Xanafide), Volorexin. Furthermore, these signatures may or may not include pChk2, pH2AX, cleaved caspase, or cleaved PARP, but may include other other activatable elements, which may be selected from the list: phosphorylated or cleaved forms of proteins selected from Jak1, Jak2, SOCs, Rac, Rho, Cdc42, Ras-GAP, Vav, Tiam, Sos, Db1, Nck, Gab, PRK, SHP1, and SHP2, SHIP1, SHIP2, sSHIP, PTEN, Shc, Grb2, PDK1, SGK, Akt1, Akt2, Akt3, TSC1,2, Rheb, mTor, 4EBP-1, p70S6Kinase, S6, LKB-1, AMPK, PFK, Acetyl-CoAa Carboxylase, DokS, Rafs, Mos, Tpl2, MEK1/2, MLK3, TAK, DLK, MKK3/6, MEKK1,4, MLK3, ASK1, MKK4/7, SAPK/JNK1,2,3, p38s, Erk1/2, Syk, Btk, BLNK, LAT, ZAP70, Lck, Cb1, SLP-76, PLCγ1, PLCγ 2, STAT1, STAT 3, STAT 4, STAT 5, STAT 6, FAK, p130CAS, PAKs, LIMK1/2, Hsp90, Hsp70, Hsp27, SMADs, Rel-A (p65-NFKB), CREB, Histone H₂B, HATs, HDACs, PKR, Rb, Cyclin D, Cyclin E, Cyclin A, Cyclin B, P16, p14Arf, p27KIP, p21CIP, Cdk4, Cdk6, Cdk7, Cdk1, Cdk2, Cdk9, Cdc25, A/B/C, Ab1, E2F, FADD, TRADD, TRAF2, RIP, Myd88, BAD, Bcl-2, Mcl-1, Bcl-XL, Caspase 2, Caspase 3, Caspase 6, Caspase 7, Caspase 8, Caspase 9, IAPs, Smac, Fodrin, Actin, Src, Lyn, Fyn, Lck, NIK, IκB, p65(RelA), IKKα, PKA, PKC [symbol] , PKC [symbol] , PKC [symbol] , PKCδ, CAMK, Elk, AFT, Myc, Egr-1, NFAT, ATF-2, Mdm2, p53, DNA-PK, Chk1, Chk2, ATM, ATR, \tilde{β}catenin, CrkL, GSK3α, GSK3β, and FOXO; or phosphorylation of forms of one or more proteins selected from the group Akt1, Akt2, Akt3, SAPK/JNK1,2,3, p38s, Erk1/2, Syk, ZAP70, Btk, BLNK, Lck, PLCγ, PLCγ 2, STAT1, STAT 3, STAT 4, STAT 5, STAT 6, CREB, Lyn, p-S6, Cb1, NF-κB, GSK3β, CARMA/Bcl10 and Tcl-1.

### Pathway Profiling

In some embodiments, the invention provides methods for measuring activity at multiple steps in a signaling pathway. For disease cells with aberrant signaling activity, these methods may be used to determine the step or steps of the pathway at which signaling is disrupted. Identification of the disrupted steps may enable the selection of targeted therapeutics. For example, the methods of the invention can distinguish between DNA damage-dependent cell cycle arrest and DNA damage-independent cell cycle arrest, and further can identify the stage of cell cycle arrest:

In response to double-stranded DNA breaks, the ataxia telangiectasia mutated (ATM) kinase is activated through autophosphorylation, and induces cell cycle arrest by acting on multiple targets (for review, see Riches, L. C., et al. Early events in the mammalian response to DNA double-strand breaks. Mutagenesis. (2008) 23:331-9). ATM is one of several kinases known to directly phosphorylate the histone variant H2AX, which nucleates a DNA damage response complex. ATM activity also phosphorylates Chk2 and p53. The Chk2 checkpoint kinase is central to transducing the DNA damage signal and p53, thus regulating both cell cycle arrest and apoptosis. Cyclin B1 is produced during the G2 phase of the cell cycle and its accumulation drives the cell cycle into M phase. Therefore, low levels of Cyclin B1 mark G1 and S phases, high levels mark G2 phase, and higher levels mark M phase. Histone H3 (S28) becomes phosphorylated in M phase, making p-Histone H3(S28) a useful marker of cells in M phase and not G2. It is also possible to monitor the G2/M phases of the cell cycle by measuring the phosphorylation status of Cdk1, previously known as Cdc2, a cyclin-dependent protein kinase that controls the cell cycle entry from G2 to M phase. An inhibitory phosphorylation on Cdk1 is removed by CDC25C in the M phase transition, allowing G2 cells to be distinguished from M cells based on levels of p-Cdk1. Treating myeloid cells with Mylotarg produces DNA damage by activated calicheamicin, resulting in cell cycle arrest. Treating cells with the microtubule polymerization inhibitor Nocodazole induces cells cycle arrest in M phase without DNA damage. Multiparameter flow cytometry can be used to measure the levels of activated DNA damage response elements, p-H2AX(5139), p-p53(S15), p-Chk2(T68), and p-ATM(51981) and markers of cell cycle arrest, p-Cdk1 (G2 phase), Cyclin B1 (G2-M), p-H3 (M) in single cells in response to Mylotarg.

For example, in Mylotarg-resistant cells, Ramos cells, GDM-1 or KG-1 cells, Mylotarg treatment does not induce changes in levels of p-H2AX(5139), p-p53(S15), p-Chk2(T68), and p-ATM(S1981), indicating failure to undergo a DNA damage response. Furthermore, treating these cells with Mylotarg does not largely increase the proportion of populations of cells arrested in the cell cycle, as indicated by the levels of p-Histone H3(S28), p-CDK1, and CyclinB1 accumulation. However, in Mylotarg-sensitive cells, for example the U937 cell line, Mylotarg treatment induces increased levels of p-H2AX(S139), p-Chk2(T68), and p-ATM(S1981), indicating a DNA damage response. In the U937 cell line, 24 hours of Mylotarg exposure induced cell populations with high levels of Cyclin B1 and low levels of p-Histone H3(S28), and populations with high p-Cdk1 and high Cyclin B1, indicating arrest at the G2/M checkpoint. In contrast, exposing the U937 cell line to Nocodazole for 24 hours induces cell populations with increased levels of Cyclin B1, low p-Cdk1, and high p-histone H3 (S28), indicating that Nocodozole induces arrest in M rather than G2. Exposing U937 cells to Mylotarg for 48 hours results in a major increase in cell populations with high levels of Cyclin B1 and low levels of p-Histone H3(S28), and high Cyclin B1, indicating arrest at the G2/M checkpoint, and a minor cell population with increased p-histone H3(S28), indicating minor arrest in M phase under these conditions. These results show that in some embodiments, the invention can demonstrate under two or more sets of conditions that two compounds have distinct mechanisms of action on cell signaling.

Following exposure to Mylotarg, U937 cell line undergoes cell cycle arrest and a DNA damage response. These data suggest that Mylotarg can induce cell cycle arrest and DNA damage response through a p-53 independent mechanism.

Without intending to be limited to any theory, this example illustrates that the methods of the invention may identify the specific stage of the cell cycle at which the cell cycle arrests in response to treatment with a modulator. Furthermore, it illustrates that the methods of the invention can be used to distinguish DNA-damage induced cell cycle arrest from cell cycle arrest without DNA damage, and also determine whether a DNA damage response is linked to apoptosis. One skilled in the art will appreciate that in other embodiments, the methods of the invention may be used to identify linkage between other node states, for example, between p53 phosphorylation and apoptosis. In other embodiments, the methods of the invention may be used to identify the specific step in a signaling pathway at which signaling is disrupted, for example in a disease, or by treatment with a modulator. In other embodiments, the methods of the invention may be used to identify the effects of modulator treatment on specific steps in a signaling pathway, including, but not limited to pathways disrupted in disease.

### Kits

In some embodiments the invention provides kits. In some embodiments, the invention provides kits for the classification, diagnosis, prognosis of a condition and/or prediction of outcome after administering a therapeutic agent to treat the condition, the kit comprising one or more modulators, inhibitors, specific binding elements for signaling molecules, and may additionally comprise one or more therapeutic agents. The kit may further comprise a software package for data analysis of the cellular state and its physiological status, which may include reference profiles for comparison with the test profile and comparisons to other analyses as referred to above. The kit may also include instructions for use for any of the above applications.

Kits provided by the invention may comprise one or more of the state-specific binding elements described herein, such as phospho-specific antibodies. A kit may also include other reagents that are useful in the invention, such as modulators, fixatives, containers, plates, buffers, therapeutic agents, instructions, and the like.

In some embodiments, the kit comprises one or more of antibodies which recognize dynamic state changes, protein modification, phosphorylation, methylation, acetylation, ubiquitination, SUMOylation, or cleavage of the proteins selected from the group consisting of PI3-Kinase (p85, p110a, p110b, p110d), Jak1, Jak2, SOCs, Rac, Rho, Cdc42, Ras-GAP, Vav, Tiam, Sos, Db1, Nck, Gab, PRK, SHP1, and SHP2, SHIP1, SHIP2, sSHIP, PTEN, Shc, Grb2, PDK1, SGK, Akt1, Akt2, Akt3, TSC1,2, Rheb, mTor, 4EBP-1, p70S6Kinase, S6, LKB-1, AMPK, PFK, Acetyl-CoAa Carboxylase, DokS, Rafs, Mos, Tpl2, MEK1/2, MLK3, TAK, DLK, MKK3/6, MEKK1,4, MLK3, ASK1, MKK4/7, SAPK/JNK1,2,3, p38s, Erk1/2, Syk, Btk, BLNK, LAT, ZAP70, Lck, Cb1, SLP-76, PLCγ1, PLCγ 2, STAT1, STAT 3, STAT 4, STAT 5, STAT 6, FAK, p130CAS, PAKs, LIMK1/2, Hsp90, Hsp70, Hsp27, SMADs, Rel-A (p65-NFκB), CREB, Histone H2B, HATs, HDACs, PKR, Rb, Cyclin D, Cyclin E, Cyclin A, Cyclin B, P16, p14Arf, p27KIP, p21CIP, Cdk4, Cdk6, Cdk7, Cdk1, Cdk2, Cdk9, Cdc25, A/B/C, Ab1, E2F, FADD, TRADD, TRAF2, RIP, Myd88, BAD, Bcl-2, Mcl-1, Bcl-XL, Caspase 2, Caspase 3, Caspase 6, Caspase 7, Caspase 8, Caspase 9, IAPs, Smac, Fodrin, Actin, Src, Lyn, Fyn, Lck, NIK, IκB, p65(RelA), IKKα, PKA, PKC [symbol] , PKC [symbol] , PKC [symbol] , PKCδ, CAMK, Elk, AFT, Myc, Egr-1, NFAT, ATF-2, Mdm2, p53, DNA-PK, Chk1, Chk2, ATM, ATR, \tilde{β}catenin, CrkL, GSK3α, GSK3β, and FOXO. In some embodiments, the kit comprises one or more of the phospho-specific antibodies specific for the proteins selected from the group consisting of Erk, Syk, Zap70, Lck, Btk, BLNK, Cb1, PLCγ2, Akt, RelA, p38, S6. In some embodiments, the kit comprises one or more of the phospho-specific antibodies specific for the proteins selected from the group consisting of Akt1, Akt2, Akt3, SAPK/JNK1,2,3, p38s, Erk1/2, Syk, ZAP70, Btk, BLNK, Lck, PLCγ, PLCγ 2, STAT1, STAT 3, STAT 4, STAT 5, STAT 6, CREB, Lyn, p-S6, Cb1, NF-κB, GSK3β, CARMA/Bcl10, p-Chk1, p-Chk2, p-ATM, p-H2AX and Tcl-1. In some embodiments, the kit comprises one or more of the specific antibodies specific for the proteins selected from the group consisting of PARP, caspase 3 and p-53.

Kits provided by the invention may comprise one or more of the modulators described herein. In some embodiments, the kit comprises one or more modulators selected from the group consisting of PMA, SCF and IL-6.

The state-specific binding element of the invention can be conjugated to a solid support and to detectable groups directly or indirectly. The reagents may also include ancillary agents such as buffering agents and stabilizing agents, e.g., polysaccharides and the like. The kit may further include, where necessary, other members of the signal-producing system of which system the detectable group is a member (e.g., enzyme substrates), agents for reducing background interference in a test, control reagents, apparatus for conducting a test, and the like. The kit may be packaged in any suitable manner, typically with all elements in a single container along with a sheet of printed instructions for carrying out the test.

Kits provided by the invention may comprise one or more assays to determine the expression and/or function of one or more drug transporters.

In some embodiments, the kits of the invention enable the detection of activatable elements by sensitive cellular assay methods, such as IHC and flow cytometry, which are suitable for the clinical detection, prognosis, and screening of cells and tissue from patients, such as leukemia patients, having a disease involving altered pathway signaling.

Such kits may additionally comprise one or more therapeutic agents. The kit may further comprise a software package for data analysis of the physiological status, which may include reference profiles for comparison with the test profile.

Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. Kits described herein can be provided, marketed and/or promoted to health providers, including physicians, nurses, pharmacists, formulary officials, and the like. Kits may also, in some embodiments, be marketed directly to the consumer.

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes. All references cited herein are expressly incorporated by reference in their entireties.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

### Example 1

### Materials and Methods

The present illustrative example represents how to analyze cells in one embodiment of the present invention. There are several steps in the process, such as treatment with modulator, incubation with antibodies, and processing sample for acquisition of cells on the cytometer. Treatment with modulator(s) can start with thawed cryopreserved cells and end with cells fixed in PFA and permeabilized in methanol. Then the cells can be incubated with an antibody directed to a particular protein of interest and then analyzed using a flow cytometer. A protocol similar to the following was used to analyze AML cells from patient samples. The materials and methods described in this example can be used and/or were used in the other examples described below.

Materials: In this example, the following materials are used: Phosphate Buffered Saline (PBS) (MediaTech); Thawing media (PBS-CMF+10% FBS+2 mM EDTA); 70 um Cell Strainer (BD); 1 uL anti-CD45 Alexa 700 (Invtrogen) per sample; 1 ug/mL propidium iodide (PI) solution (Sigma) (7-AAD or an equivalent viability dye can also be used); RPMI+1% FBS; Media A (RPMI+1% FBS+1× Penn/Strep); Live/Dead Reagent, Amine Aqua (Invitrogen); 2 mL, 96-Deep Well, U-bottom polypropylene plates (Nunc); 300 uL 96-Channel Extended-Length D.A.R.T. tips for Hydra (Matrix); 16% Paraformaldehyde (Electron Microscopy Sciences); 100% Methanol (EMD); Transtar 96 dispensing apparatus (Costar); Transtar 96 Disposable Cartridges (Costar, Polystyrene, Sterile); Transtar reservoir (Costar); Foil plate sealers.

Cell thawing and live/dead staining: Cells are thawed_in a 37° C. water bath, gently resuspend in the vial, and transferred to a 15 mL conical tube. The 15 mL tube is centrifuged at 930 RPM (200×g) for 8 minutes at room temperature. The supernatant is then aspirated, and the pellet is gently resuspended in 1 mL media A. The cell suspension is filtered through a 70 um cell strainer into a new 15 mL tube. The cell strainer is then rinsed with 1 mL media A and another 12 mL of media A into the 15 mL tube. Collected cells are then mixed into an even suspension. A 20 μl, aliquot of this suspension is immediately transferred into a 96-well plate containing 180 μl, PBS+4% FBS+CD45 Alexa 700+PI to determine cell count and viability post spin. After the determination, the 15 mL tubes are centrifuged at 930 RPM (200×g) for 8 minutes at room temperature. The resulting supernatant is aspirated, and the cells are gently resuspended in 4 mL PBS+4 μl, Amine Aqua (another equivalent viability dye can be used instead) and incubated for 15 min in a 37° C. incubator. 10 mL RPMI+1% FBS are added to the cell suspension, followed by inversion mixing. The 15 mL tubes are centrifuged at 930 RPM (200×g) for 8 minutes at room temperature. The stained cells are then resuspended in Media A at the desired cell concentration (1.25×10⁶/mL).
a. Samples with low numbers of cells (<18.5×10⁶) are resuspend in up to 15 mL of media.
b. For samples with high numbers of cells (>18.5×10⁶), the volume is raised to 10 mL with media A and the desired volume is transferred to a new 15 mL tube, adjusting the cell concentration to 1.25×10⁶ cells/mL.
   1.6 mL of the above cell suspension are then transferred into wells of a multi-well plate, from which 80 ul are distributed into each well of a subsequent plate. Plates are covered with a lid (Nunc) and placed in a 37° C. incubator for 2 hours to rest.

Treatment of Cells with a Modulator: A concentration for each stimulant that is five fold (5×) more than the final concentration is prepared using Media A as diluent. The 5× stimulants are arrayed in a standard 96 well v-bottom plate that correspond to the wells on the plate with cells to be stimulated. Fixative is prepared by dilution of stock 16% paraformaldehyde with PBS to a concentration that is 1.5×, then placed in a 37° C. water bath. Once the plated cells have completed their incubation, the plate(s) are taken out of the incubator and place in a 37° C. water bath next to the pipette apparatus. Prior to addition of stimulant, each plate of cells is taken from the water bath and gently swirled to resuspend any settled cells. The stimulant is pipetted into the cell plate, which is then held over a vortexer set to “7” and mixed for 5 seconds, and followed by the return of the deep well plate to the water bath.

Fixing Cells: 200 μL of the fixative solution (final concentration is 1.6%) is dispensed into wells and then mixed on the titer plate shaker on high for 5 seconds. The plate is then covered with foil sealer and floated in 37° C. water bath for 10 min, followed by a 6 min spin at 2000 rpm at room temperature. Supernatant is removed using a 96 well plate aspirator (VP Scientific), and cells are resuspended in the residual volume by vortexing, achieving pellet dispersion prior to the methanol step (see cell permeabilization) to avoid clumping. Cell Permeabilization: Permeability agent (such as methanol) is added slowly and while the plate is vortexing, securely placed on the titer plate shaker set to shake at the highest setting. Using a pipetter, 0.6 mLs of 100% methanol is added to plate wells. Plate(s) are placed on ice until this step has been completed for all plates, after which plates are covered with a foil seal using the plate roller to achieve a tight fit. At this stage the plates may be stored at −80° C.

Incubation of cells with antibodies and processing for cytometry: Cells are pelleted at 2000 rpm for 5 minutes. A vacuum aspirator is used to remove the resulting supernatant, and the plate is vortexed on the plate-vortexer for 5 to 10 seconds. Cells are washed with 1 mL FACS/stain buffer (PBS+0.1% Bovine serum albumen (BSA)+0.05% Sodium Azide). The plate is then spun, vortexed, and aspirated as above, which can be further repeated if desired. To 2 rows of cells at a time, 50 μl of FACS/stain buffer with the desired, previously optimized antibody cocktail, is added (staining volume can vary) followed by agitation. Samples are covered and incubated on plate shaker for 30 minutes at room temperature (staining duration and temperature can vary). During this incubation, the compensation plate is prepared: In a standard 96-well V-bottom plate, 20 μL of “diluted bead mix” (1 mL FACS buffer+1 drop anti-mouse Ig Beads+1 drop negative control beads) is added per well. Each well receives 5 μL of 1 fluorophor conjugated control IgG (such as Alexa488, PE, Pac Blue, Aqua, Alexa647, Alexa700). For the Aqua well, 200 uL of Aqua−/+ cells are added. Following these additions, the compensation plate is incubated at room temperature for 10 minutes, followed by a wash with 200 uL FACS/stain buffer, centrifugation at 2000 rpm for 5 minutes, and removal of supernatant. This wash, centrifugation, and removal step is then repeated, followed by resuspension in 200 uL FACS/stain buffer and transfer to a U-bottom 96-well plate. (As an alternative to the compensation plate, cells such as PBMC can be used for single color controls or fluorescence minus one (FMO) controls. Also, machine cytometers can be standardized with predefined voltages and compensation settings for specific combinations of fluorophores and Quality Controlled daily.) To the cell sample plate, after the above 30 minute incubation, 1 mL of FACS/stain buffer is added per well and cover applied, followed by a 5 minute incubation on a plate shaker at room temperature. Cells are centrifuged, aspirated, and vortexed as above. 1 mL of FACS/stain buffer is again applied to each well, followed by the application of a cover, and incubation for 5 minutes on a plate shaker. Centrifugation, aspiration, and vortexing are again repeated as above. Cells are resuspended in 75 uL of FACS/stain buffer (resuspension volume can vary), and the plate is covered and incubated on a plate shaker for 5 minutes at room temperature. Cells are then analyzed using a flow cytometer, such as a LSRII (Becton Dickinson), with a high throughput screening (HTS) 96-well plate reader, all wells selected, and the following Loader Settings: 2 uL/sec flow rate; 40 uL sample volume; 250 uL/sec mixing speed; number of mixes set to 5; 800 uL wash volume; and standard mode. When the plate has completed, a batch analysis in performed to ensure there are no clogs.

### Example 2

### Profile for Mylotarg Resistance

Mvlotarg: Gemtuzumab ozogamicin (G0), a humanized IgG4 anti-CD33 monoclonal antibody conjugated to n-acetyl-γ-calicheamicin dimethyl hydrazide, is indicated for the treatment of patients with CD33 positive AML in first relapse who are 60 years or older and who are not considered candidates for cytotoxic therapy (Bross et al., Clin. Can. Research (2001) 7:1490-1496, Sievers et al., J. Clin. One. (2001) 19:3244). Many individual steps make-up Mylotarg's mechanism of action (MOA) including but not limited to binding of the monoclonal antibody component of the drug conjugate to the CD33 antigen expressed by leukemic cells, followed by cellular internalization, hydrolytic release of calicheamicin, DNA damage and eventual cell apoptosis. Key parameters involved in Mylotarg's MOA are: CD33 expression, drug transporter function, drug transporter expression, internalization mechanisms for the drug Mylotarg, intracellular hydrolysis (pH change), redox potential of environment, signaling pathways, DNA damage response, and apoptosis. (Giles et al., Cancer (2003) 98:2095, Lindberger., Leukemia (2005) 19:176). In contrast to prior studies where each step in Mylotarg's MOA is measured individually, the objective of the current example is to measure many of these steps simultaneously in individual leukemic cells by multiparameter flow cytometry (Irish et al., Nature Rev. Cancer (2006) 6:146, Danna and Nolan, Current Op. in Chem. Biol. (2006) 10:20, Nolan., (2007) Nat. Chem. Biol. 3:187). The aim of the example is to define which step (or combination of steps) in the Mylotarg MOA cascade is associated with the ability of a cell to respond or not respond to Mylotarg treatment. In addition, modulated signaling and protein expression can be used to derive a proteomic profile that can be combined with measurements of Mylotarg's MOA to predict responsiveness to the drug. Initial studies are performed in leukemic cell lines, with subsequent expansion into AML patient samples.

Cell lines: HL-60 and U937 are sensitive for arrest at G2/M and apoptosis in response to Mylotarg. THP-1 is sensitive for arrest at G2/M but not apoptosis in response to Mylotarg. KG-1 and GDM-1 are resistant to cell cycle arrest and apoptosis after Mylotarg treatment. MEC-2 and Ramos are CD33- and can serve as negative controls.

CD33 expression levels: Despite a wide inter-patient variability of CD33 expression on AML blasts, reports to date have failed to show a correlation at the single patient level between CD33 expression levels and response to Mylotarg treatment (Sievers et al., J. Clin. One. (2001) 19:3244, Lindberger., Leukemia (2005) 19:176). Subpopulations within the CD33 blast population may have different sensitivities to the drug. CD33 levels can be determined on unfixed cells and fixed and permeabilized cells allowing the CD33 antibody to be used as a gating agent. In its capacity as a gating agent, CD33 antibody can be used to determine intracellular signaling within the CD33 expressing population of cells and to monitor changes in CD33 protein levels in response to Mylotarg treatment. In some cases, when the CD33 antibody component of Mylotarg blocks the binding of the gating CD33 antibody, alternative antibodies that recognize different epitopes on CD33 would allow gating of CD33-expressing cells after Mylotarg exposure and could be used to quantify total CD33 levels and measure CD33 degradation.

Drug transporter expression and function: Previous analyses have shown that drug transporter activity, specifically from Pgp and MRP1, was associated with a worse clinical outcome to Mylotarg mono-therapy (Walter et al. Blood (2003) 102:1466, Lindberger., Leukemia (2005) 19:176, Giles et al., Cancer (2003) 98:2095, Walter et al., Blood (2007) 109:4168). However, treatment of Pgp/MRP positive AML samples with transporter inhibitors enhanced Mylotarg cytotoxicity in some but not all of the samples (Walter et al. Blood (2003) 102:1466). Thus, since expression of drug transporters does not necessarily correlate with Mylotarg activity, alternative mechanisms of resistance are proposed for lack of responsiveness (refractoriness or resistance) to Mylotarg. In this example multiparameter flow cytometry is applied to measure drug transporter expression and function simultaneously with modulated signaling and/or measurements directed toward Mylotarg's MOA. These determinations are made first in cell lines and then in AML samples to determine whether levels of expression and function differ within subpopulations of cells.

Determination of transporter expression: Levels of transporter expression are determined using the following reagents: ABCC1/MRP1-PE (from R and D systems, Minneapolis, Minn., which recognizes intracellular epitope), BCRP1/ABCG2-APC (From R and D systems and recognizes extracellular epitope) and Pgp/MDR1/ABCB1-PE (from Millipore, Billerica, Mass., or Beckman Coulter and recognizes extracellular epitope). Simultaneous measurements of drug transporter and CD33 expression levels are made in cell lines and in AML patient samples. In addition, simultaneous measurements are made using multiparametric flow cytometry for transporter expression and function with changes in surface markers and intracellular signaling readouts in response to modulators including such as cytokines, growth factors and chemokines.

Determination of transporter functional activity: Transporter activity is measured in subpopulations gated for CD45, CD11b, CD34, CD38, and other surface markers including drug transporters themselves. Fluorescent dyes including calcein-acetoxymethyl ester in the presence or absence of inhibitors cyclosporin or PSC-833 for MDR1, reserpine for BCRP-1 and MK571 or probenecid for MRP-1) are used to evaluate the contribution of each transporter to drug efflux

For example, experiments using DIOC2(3) fluorescent dye and the MDR-1 inhibitor PSC-833 demonstrate that Mylotarg resistant KG-1 cells efflux fluorescent DIOC2(3) and this MDR1 drug pump activity can be blocked by co-incubation with an MDR1 inhibitor such PSC-833. In contrast, Mylotarg sensitive U937 cells do not efflux fluorescent DIOC2(3) and display no MDR1 drug pump activity (FIG. 5). Evaluation of Drug Pump Activity could be performed using the reagents as described in Table 2 and Table 3.

TABLE 2

| Antibody | Color | Clone | Cat# | Lot# | Isotype | Vendor | Concentration | Final Antibody amount |
|---|---|---|---|---|---|---|---|---|
| MDR1 | PE | UIC2 | IM237 | 22 | IgG2a | Beckman | 25 ug/ml | 250-500 ng |
| | | | OU | | | Coulter | | |
| Isotype | PE | 7T4- | A0914 | 11 | IgG2a | Beckman | 6.25 ug/ml | 250-500 ng |
| Control | | 1F5 | 21 | | | Coulter | | |

TABLE 3

| Reagent | Cat# | Lot# | Vendor | Stock Concentration | Final Concentration |
|---|---|---|---|---|---|
| PSC-833 | SB-PSC833-4 | 23.Jul.2009 | Xenotech | 2 mM | 2 uM |
| DiOC2 | 320684 | 52896MH | Sigma-Aldrich | 1 mg/ml | 1.5 ng/ml |

| | |
|---|---|
| Cell Line Media | RPMI + 10% FBS, 0.45% Glucose, 0.5 uM Sodium Pyruvate, Pen/Strep, L-Glut |
| Assay Media | RPMI 10% FBS, Pen/Strep, L-Glut (or equivalent Aqueous-based buffer) |
| Assay Buffer | 1x DPBS, 0.5% BSA (or equivalent Aqueous-based buffer) |

Of note the resuspension buffer contains a drug pump inhibitor, in this case PSC-833 (PSC833) to inhibit efflux of DIOC2(3) fluorescent dye during the acquisition of data on the cytometer. This converts the assay into a scalable method for a high-throughput format with large numbers of samples. This is significant because without the addition of PSC-833 or alternative drug transporter/pump inhibitor in the resuspension buffer, cells can continue to efflux the DIOC2(3) dye because the acquisition of data on the cytometer is typically at ambient temperature (for example 15±30° C.) where drug pumps can still be active.

a. Cell Preparation

Cells are thawed, counted and resuspended at 0.222×10⁶/ml in RPMI 10% FBS. This example uses 100,000 cells in 450 microliters aliquoted into 6 columns of a 96 well Deep-Well “Cell Plate”. However, the experimental setup is not limited to using 100,000 cells/condition. KG-1 cells are included as a control since they express functional MDR-1. Tables 4-8 shows the general experimental setup (Table 4) and master plates for high throughput addition of Inhibitors (Table 5), Fluorescent Dye (Table 6), Efflux Buffers (Table 7), and Antibody (Table 8).

TABLE 4
General Plate Setup:

| | | Conditions: | | | | | |
|---|---|---|---|---|---|---|---|
| | | 1 | 2 | 3 | 4 | 5 | 6 |
| | | Antibody: | | | | | |
| | | MDR-1 Stain | | Isotype Control | | MDR-1 | Isotype |
| | Treatment: | Dye | Dye + Inhibitor | Dye | Dye + Inhibitor | Untreated | |
| Samples: | Donor A | | | | | | |
| | Donor B | | | | | | |
| | Donor C | | | | | | |
| | Donor D | | | | | | |
| | Donor E | | | | | | |
| | Donor F | | | | | | |
| | U937/ | | | | | | |
| | GDM-1 | | | | | | |
| | KG-1 | | | | | | |

TABLE 5
Inhibitor Plate Setup:

| | | Conditions: | | | | | |
|---|---|---|---|---|---|---|---|
| | | 1 | 2 | 3 | 4 | 5 | 6 |
| | Treatment: | Mock | Inhibitor | Mock | Inhibitor | Mock | |
| Samples: | Donor A | | | | | | |
| | Donor B | | | | | | |
| | Donor C | | | | | | |
| | Donor D | | | | | | |
| | Donor E | | | | | | |
| | Donor F | | | | | | |
| | U937/ | | | | | | |
| | GDM-1 | | | | | | |
| | KG-1 | | | | | | |

TABLE 6
Dye Plate Setup:

Conditions:

| | | | | | |
|---|---|---|---|---|---|
| 1 | 2 | 3 | 4 | 5 | 6 |

| | | |
|---|---|---|
| Treatment: | Dye | Mock |

| | |
|---|---|
| Samples: | Donor A |
| | Donor B |
| | Donor C |
| | Donor D |
| | Donor E |
| | Donor F |
| | U937/ |
| | GDM-1 |
| | KG-1 |

TABLE 7
Efflux Plate Setup:

| | | Conditions: | | | | | |
|---|---|---|---|---|---|---|---|
| | | 1 | 2 | 3 | 4 | 5 | 6 |
| | Treatment: | Mock | Inhibitor | Mock | Inhibitor | Mock | |
| Samples: | Donor A | | | | | | |
| | Donor B | | | | | | |
| | Donor C | | | | | | |
| | Donor D | | | | | | |
| | Donor E | | | | | | |
| | Donor F | | | | | | |
| | U937/ | | | | | | |
| | GDM-1 | | | | | | |
| | KG-1 | | | | | | |

TABLE 8
Antibody Plate Setup:

| | | Conditions: | | | | | |
|---|---|---|---|---|---|---|---|
| | | 1 | 2 | 3 | 4 | 5 | 6 |
| | Antibody: | MDR-1 | | Isotype Control | | MDR-1 | Isotype |
| Samples: | Donor A | | | | | | |
| | Donor B | | | | | | |
| | Donor C | | | | | | |
| | Donor D | | | | | | |
| | Donor E | | | | | | |
| | Donor F | | | | | | |
| | U937/ | | | | | | |
| | GDM-1 | | | | | | |
| | KG-1 | | | | | | |

b. Drug Treatments: Untreated (no Dye or Inhibitor), Dye alone, Dye+inhibitor

In this example, the Fluorescent Dye is DIOC(2)₃ and the inhibitor is PSC-833.

10× Inhibitor or 10× Mock Solutions are prepared by the following protocols:
10×PSC-833. Stock Conc=2 mM=2000 uM. 10× Conc=20 uM (1:100).
   Combine 25 uL stock with 2475 uL DPBS/0.5% BSA
   Note Final Conc DMSO=0.1%
   Could likely use a range of PSC-833 Concentrations not limited to 2 uM.
   This experiment uses Final Concentration of 2 uM
10×DMSO buffer (mock)
   Combine 25 uL DMSO with 2475 uL DPBS/0.5% BSA

65 ul per well of 10× buffers are aliquoted to 96 well V bottom Inhibitor Plate. Table 5 above shows the general format of the Inhibitor plate. The Inhibitor plate is sealed and stored at 37° C. in incubator until use. The remainder 10× Inhibitor or Mock buffers are stored at 4 C for making efflux buffers.

Using a 96-well pipettor such as the Rainin liquidator, or a Matrix Hydra, or equivalent, 50 ul 10× DMSO buffer or 10×PSC-833 are aliquoted from Inhibitor Plate to Cell Plate, mixed 3 times with the liquidator, and vortexed for 5 sec at 1000 rpm on digital vortexer. The Cell Plate is then incubated for 15 min in a 37° C. incubator. Note: Other pipettors that are not 96-well format may also be used in this protocol

c. Dye Load

26×DIOC2 Buffer is prepared using the following protocol.
Stock 1 Conc=1000 ug/mL=10̂6 ng/mL=666666×
Stock 2 Conc=3900 ng/mL (1:256 dilution of Stock 1)=2600×
Stock 3 Conc=39 ng/mL (1:100 dilution of Stock 2)=26×
Final Conc=1.5 ng/mL
   Prepare Stock 2:
   Combine 4 uL Stock 1 with 1022 uL DPBS/0.5% BSA
   Prepare Stock 3:
   Combine 46 uL Stock 2 with 4554 uL DPBS/0.5% BSA

This experiment uses Final Concentration 1.5 ng/mL of DIOC2. However, the functional assay could potentially use a range of concentration of DIOC2, not limited to 1.5 ng/mL. 40 ul per well of Stock 3 (26×DIOC2 Solution) or DPBS/0.5% BSA are aliquoted to a 96 well V bottom Dye Plate. Table 6 shows the general format of the Dye Plate. The Dye Plate is stored at 37° C. until use.

After Cell Plates have incubated with inhibitors for 15′ the Dye is added to Cell Plates. The Dye Plate and Cell Plate are removed from 37° C. incubation 30 sec prior to addition of dye. Using a 96-well pipettor such as the Rainin Liquidator, 20 ul dye buffer are aliquoted per well from Dye Plate to Cell Plate. The mixtures are mixed 3× with liquidator and vortexed for 5 sec at 1000 rpm using a digital vortexer. The mix is incubated at 45 min at 37° C. in incubator. After 45 minutes, Cell Plate is spun at 400×g for 5 min at RT, aspirated, and vortexed at 2000 RPM 5 sec to resuspend cells.

d. Efflux

The efflux buffers are prepared in Reagent Reservoir using the following protocols:
For Inhibitor Efflux Buffer:
   Combine 1 mL 10×PSC-833 with 9 mL RPMI 10% FCS
For Mock Efflux Buffer:
   Combine 2 mL 10× Mock with 18 mL RPMI 10% FCS

520 ul per well of Efflux Buffers are aliquoted into Efflux Plate 96 deep well plate. Table 7 shows the general format of the Efflux Plate. The Efflux Plate is stored at 37° C. until use.

After cells are aspirated, vortexed, Using liquidator, 470 ul (3×157 ul on liquidator) Efflux Buffers are aliquoted from Efflux Plate to Cell Plate. The mixture is mixed 3× with liquidator and vortexed for 5 sec at 1000 rpm using a digital vortexer. The plate is incubated for 20 min at 37° C. in incubator.

d. Incubation with Antibody

The protocol for incubation with antibody is all done on ice and kept cold. After the efflux step, Cells are spun 400×g 5 min. The supernatant is aspirated, and vortexed 5 sec at 2000 rpm to resuspend. The Cell Plate is put on ice. The centrifuge is set to 4 C. For Fc Blocking, human IgG solution is prepared and added to wells using 1 ug of Human IgG per well. This assay could use a range of concentrations not limited to 1 ug/well to block. In this example, 10 uL Human IgG block solution (100 ug/mL) are pippetted to Cell Plate using a 24-well Pipettor. The mixture is mixed 3× and vortexed 5 sec at 2000 rpm. The plate is then incubated 5′ on ice.

For antibody incubations MDR-1 and Isotype mixtures are prepared. These mixtures can have surface markers such as CD34, CD38, CD45, CD33, CD15, and CD11b in combination with either Isotype Control Antibody or MDR-1 Antibody. Other surfaces markers could be used in this assay. 120 uL of each is aliqouted to 96-well V-bottom “Master Antibody” Plate according to Table 8 above.

100 uL Antibody mixtures are pipetted to appropriate wells of Cell Plates, mixed 3× and vortexed 5 sec at 2000 rpm. The plate is incubated 30′ on ice in dark.

The Resuspension Buffer is prepared by using the following protocol:
Prepare 2 uM PSC833 Resuspension Buffer
Combine 5.18 uL Stock PSC-833 2 mM with 5.18 mL DPBS/0.5% BSA.
Keep Resuspension Buffer at Room Temp
   This assay could potentially use a different concentration of PSC833 in resuspension buffer.

After 20′, 350 ml cold DPBS/0.5% BSA is poured to a liquidator reservoir. Cells are washed with 1 ml (5×200 ul on liquidator) per well using Liquidator Filter tips. DPBS/0.5% BSA is kept cold during spin. Plate is spun at 400×g for 5 min at 4 C. The supernatant is aspirated, and vortexed 5 sec at 2000 rpm to resuspend. The cells are washed with an additional with 1 ml cold DPBS/0.5% BSA (5×200 ul on liquidator) per well using Liquidator Filter tips. The plate is spun at 400×g for 5 min at 4 C. The supernatant is aspirated, and vortexed 5 sec at 2000 rpm to resuspend.

e. Acquisition on Flow Cytometer

After final wash/aspiration, 75 ul room temp 2 uM PSC-833 Resuspension Buffer are added per well to cells using the liquidator. The entire volume is removed (aspirated residual volume+75 ul resuspension volume) and aliquoted from Cell Plate to a 96 well U-bottom Acquisition Plate. To avoid introducing bubbles, 75 uL is added then the liquidator volume is adjusted to 110 uL before transferring cells to U-bottom plates. The Acquisition Plate is then incubated at least 10 min at room temp in dark before acquisition on cytometer such as an LSRII using the High Throughput Screening (HTS) unit.

Columns 1-4 (Samples with DIOC2 added) of Acquisition Plate are acquired first, then columns 5-6 (samples without DIOC2 added) are acquired.

Redox potential of environment: Once inside the cell, the covalent link between CD33 and calicheamicin is cleaved inside lysosomes by acid hydrolysis, allowing the toxin to be released. A reactive intermediate of calicheamicin is formed through reduction, likely by intracellular glutathione, thus indicating that the cellular redox potential is critical in determining the drug efficacy (Dedon t al., Biochemistry (1993) 32:3617, Giles et al., Cancer (2003) 98:2095). In cancer, the redox environment is known to be altered due to changes in levels of reactive oxygen species (ROS) (Lu et al., Drug Discov. Today Disease Models (2007) 4:67).

Determination of ROS: Cellular levels of ROS are measured by flow cytometry. There are a number of cell permeant derivatives of reduced fluorescein and calcein that can be used as indicators for ROS. Chemically reduced and acetylated forms of 2′7′-di-chlorofluorescein (DCF) and calcein are non-fluorescent until the acetate groups are removed by intracellular esterases and the compound is oxidized (dependent on the amount of cellular ROS). The retained fluorescent molecule can then be measured along with a pre-oxidized control molecule (Molecular Probes Carlsbad, Calif. D399).

Signaling pathways—nodes and expression markers: Previous studies have identified intracellular signaling nodes in primary AML patient samples that correlate with poor response to induction therapy in vivo and also with resistance to apoptosis in vitro (Irish et al., Cell (2004)118:214). See also U.S. Ser. No. 61/085,789. In addition, expression of cellular surface markers has also been shown to correlate with poor response rate (U.S.No. 61/085,789).

Measurement of surface marker expression and evaluation of post-translational modification of cytoplasmic domains: Recent studies have shown that the CD33-related subset of sialic acid binding proteins, expressed mostly by hematpoietic and immune cells, undergo endocytosis in response to sialylated pathogens as well as other antigens. This clearance of CD33 from the cell surface can be exploited for immunotherapy in which therapeutic agents linked to CD33 ligands gain access to the cell interior upon CD33 internalization. Furthermore, post-translational modifications such as tyrosine phosphorylation within the intracellular domain of CD33 has been described to regulate the rate of its internalization. The cytoplasmic tail of CD33 has two tyrosine residues within an immunoreceptor tyrosine-based inhibitory motif (ITIM). When phosphorylated, these phospho-tyrosine residues provide docking sites for the SH2-domain-containing phosphatases (Shp-1 and Shp-2) which dephosphorylate CD33 and attenuate its internalization. Further evidence has shown that restraint of CD33 internalization can be relieved through phosphorylation of sites including but not limited to the CD33 ITIM tyrosines (Walter et al., J. Leukocyte Biology, (2008) 83:200). Reagents, including antibodies, that recognize epitopes within the CD33 ITIM motifs can be developed. Measurements of phosphorylation within these motifs, using these reagents, can be measured simultaneously with other parameters, such as drug transporter function, expression levels of Shp proteins, and signaling molecules in order to provide information about responsiveness to Mylotarg.

Measurements of Signaling nodes: Signaling nodes will be measured and correlated with in vitro apoptotic response to Mylotarg (e.g., Example 3). Cells are exposed to modulators such as G-CSF, SDF1α, SCF, Flt3-L, and hydrogen peroxide and levels of p-Erk, p-Akt, p-S6, p-STAT3, p-STAT5, p-CREB, p-PLC-γ, p-SLP SLP76, p-p38, and p-65/RelA are determined. Levels of expression for the receptors FLT3-R, SCF-R (c-Kit), SDF-R are also measured and correlated with signaling and in vitro apoptotic response to Mylotarg. Expression levels of Shp-1 and Shp-2 phosphatases are measured in cell lines and patient samples and correlated with in vitro apoptotic response to Mylotarg.

Determination of DNA damage and apoptototic response to Mvlotarg: The proposed mechanism of action of Mylotarg is to allow calicheamicin, a hydrophobic member of the endiyne family of DNA cleaving enzymes, to enter cells by linkage with a CD33 antibody and promote apoptosis (Giles et al., Cancer (2003) 98:2095). Studies have shown variability in the DNA damage responses and apoptosis with CD33 levels. (Amico et al., Blood (2003) 101:4589). Consistent with this former study, a recent communication by Goemans et al. described large inter-patient differences in cellular sensitivity to calicheamicin in 122 pediatric AML samples where Pgp expression was very infrequent. Variations in the DNA damage and apoptotic pathways may play a role in the clinical response to Mylotarg (Goemans et al., Leukemia (2008) 12:2284-5). DNA damage and apoptotic response of AML samples to Mylotarg are therefore measured (response in cell lines are also measured).

The ability of Mylotarg to promote caspase dependent and independent apoptosis in cell lines and primary AML samples is compared with etoposide and AraC/Daunorubicin. Samples are treated with each agent in the absence and presence of ZVAD or alternative pan-caspase inhibitor. DNA damage and apoptosis are evaluated by measuring levels of: p-Chk2, p-Chk1, p-ATM, p-53, cleaved caspases (Caspase 3, Caspase 8, Caspase 9), cleaved PARP, and mitochondrial Cytochrome C. Levels of activated Bak, activated Bax Bcl-2, p-BAD and cyclin B are measured as some of these markers have been described to play a role in the cytotoxicity of Mylotarg (Amico et al., Blood (2003) 101:4589, Lindberger, Leukemia (2005) 19:176).

Gating Procedure: Data acquired from the flow cytometer is analyzed with Flowjo software (Treestar, Inc), or WinList software or equivalent. The Flow cytometry data is first gated on single cells (to exclude doublets) using Forward Scatter Characteristics Area and Height (FSC-A, FSC-H) and/or by FSC-A and Side Scatter Characteristics (SSC-A). Single cells are gated on live cells by excluding dead cells that stain positive with an amine reactive viability dye (Aqua-Invitrogen) or equivalent. Live, single cells are then gated for subpopulations using scatter characteristics and antibodies that recognize surface markers, including CD33, CD45, CD11b, CD13, CD15, CD34, CD38, to identify unique subpopulations such as: CD45++, CD33− for lymphocytes; CD45++, CD33++ for monocytes and granulocytes; and CD45+, CD33+ for leukemic blasts. Signaling is analyzed by fluorescence intensities in these gated cell sub-populations, with fluorochrome-conjugated antibody panels that can simultaneously recognize several intracellular signaling molecules.

The data can then be analyzed using various metrics, including expression level of a protein, level of basal phosphorylation in the absence of a modulator, or fold change in phosphorylation (by comparing the change in phosphorylation in the absence of a modulator to the level of phosphorylation seen after treatment with a modulator), total levels of phosphorylation in the presence of a modulator, or frequency of cells that respond to a modulator, on each of the cell populations that are defined by the gates in one or more dimensions. These metrics are then organized in a database tagged by: the Donor identification (ID), plate identification (ID), well ID, gated population, stain, modulator, and other experimental conditions. These metrics, tabulated from the database, are then combined with the clinical data to identify nodes that are correlated with a pre-specified, known clinical variable of interest (for example; response or non response to therapy)

### Example 3

### Effects of Mylotarg exposure

In the following example, several observations were made regarding determinations of cell death and cell cycle, determinations of DNA damage and apoptosis, determinations of modulated intracellular signaling pathways mediated by agents including cytokines or growth factors upon Mylotarg exposure. The data is generated from cancer cell lines GDM-1, KG-1 (myeloid leukemic cell lines), and Ramos (a B cell lymphoma cell line (See U.S. Provisional Application No. 61/186,619) which are identified as Mylotarg refractory, and U937, which is identified as Mylotarg sensitive.

The experimental measurements included forward and side scatter, the use of viability dyes (amine aqua and propidium iodide (PI)), and determination of cell cycle and apoptosis by measuring DNA content with DRAQ5. Additionally, cell cycle progression was measured by determining levels of cyclin B1, phosphorylated histone H3, and phosphorylated CDK-1. All these determinations were made in freshly thawed as well as in cultured cycling cells. Results in cultured cell lines exposed in vitro to Mylotarg showed that in Mylotarg sensitive cell lines there was more cell death (determined by scatter and viability dyes as well as an increase in the sub-G1 population), G2/M arrest and an increase in cyclin B1 levels when compared to Mylotarg refractory cell lines evaluated in the same experiment (See U.S. Provisional Application No. 61/186,619).

The experiments were conducted in a similar fashion as shown above for examples 1 and 2. Cell lines were exposed to Mylotarg at clinically relevant concentrations (Dowell et al., J. Clin. Pharmacol. 2001 41:1206-1214) in vitro for 24 or 48 hrs after which they were fixed with 1.6% paraformaldehyde and permeabilized with 100% ice cold methanol. After incubation with fluorochrome-conjugated antibody panels that recognized: p-H2AX, p-Chk2, p-ATM, p-53, cleaved PARP and cleaved caspase 3, cells were processed for cytometry as described above. The data showed that Mylotarg sensitivity (where sensitivity is defined as programmed cell death or apoptosis in response to Mylotarg exposure) correlated with increased p-ATM, p-53, p-H2AX and p-Chk2 (See U.S. Provisional Application No. 61/186,619). In U937 cells, exposure with Mylotarg alone induces a significant increase in p-ATM, p-53, p-H2AX and p-Chk2 compared with GDM-1 and KG-1 cell lines where this was not the case (See U.S. Provisional Application No. 61/186,619). GDM-1 and KG-1 cells exposed to the combination of Mylotarg and cyclosporine A, an inhibitor of drug transport, resulted in increases in p-ATM, p-53, p-H2AX and p-Chk2 (See U.S. Provisional Application No. 61/186,619).

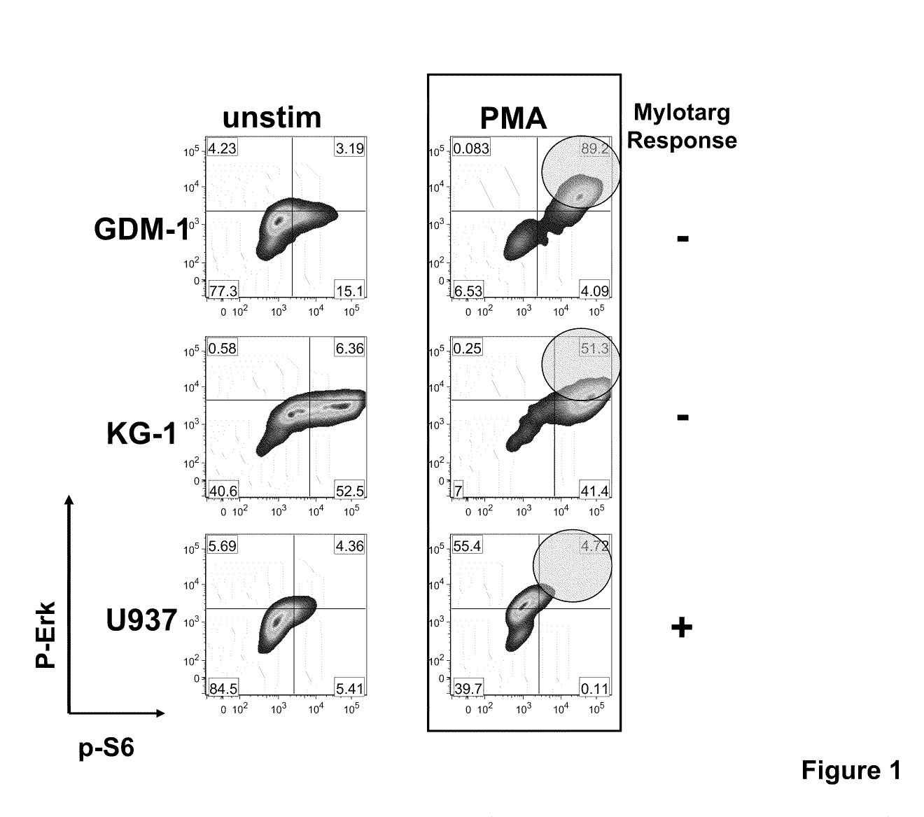

Experiments were conducted in which cell lines were exposed for 5-15 minutes to growth factors and cytokines, after which cells were fixed, permeabilized and incubated with panels of fluorochrome-conjugated antibody panels that recognized intracellular signaling molecules. After processing, cells were acquired on the cytometer. The results showed a correlation between phorbol myristic acetate (PMA)-mediated increase in p-Erk and p-S6 with Mylotarg refractoriness (FIG. 1), The data also showed correlations between an SCF-mediated increase in p-Akt and p-Erk with Mylotarg refractoriness (See U.S. Provisional Application No. 61/186,619). These data suggest that cells could be sensitized to Mylotarg by PI3k and/or Mek inhibitors. Additionally, a correlation was observed between IL-6 mediated p-Stat1 increase and Mylotarg sensitivity (See U.S. Provisional Application No. 61/186,619). The results also showed that in response to Mylotarg, sensitive cell lines identified as undergoing: G2/M arrest; increased cyclin B1 levels; increased percentage of dead cells determined by FSC/SSC and viability and DNA content dyes; increased p-H2AX and p-Chk2 without transporter inhibitor; and increased IL-6 mediated p-Stat1. In response to Mylotarg, resistant cell lines identified as: failure to undergo G2/M arrest; failure to increase cyclin B1 levels; only modest increase in cell death; minimal increase in p-H2AX and p-Chk2; increase in PMA-mediated p-S6 and SCF-mediated p-Akt, p-Erk, and p-S6 (See U.S. Provisional Application No. 61/186,619). The addition of a transporter inhibitor (cyclosporin) can increase sensitivity to Mylotarg in terms of cell death and apoptosis, but not to the level seen in sensitive cell lines (See U.S. Provisional Application No. 61/186,619).

The conditions developed for evaluating Mylotarg responsiveness in cancer cell lines can be applied to primary samples such as bone marrow mononuclear cells from healthy or AML donors or primary cells for which Mylotarg could be a potential therapeutic agent.

Table 2 delineates signaling studies that were performed to determine the mechanism of action of Mylotarg in resistant and sensitive cells.

TABLE 2
Signaling Studies in Cell Lines Selected for Different Sensitivity to Mylotarg

| Modulator | Readout | Relevant Biology |
|---|---|---|
| (-) | CD33 surface expression | Potential for Drug Binding |
| (-) | CD33 p-Y340 p-Y358*** | Drug/CD33 internalization |
| (-) | MDR-1 surface expression & Function | Calicheamicin exclusion |
| Mylotarg | p-Chk2, p-H2AX, p-p53, p-ATM | Drug induced DNA Damage |
| | | Response |
| Mylotarg | Cleaved Caspase, Cleaved PARP | Drug induced Apoptosis |
| SCF, FLT3L, PMA | p-S6, p-Erk p-Akt | PI3K, Ras/Raf pathways, |
| | | Growth & Survival Pathways |
| | | Translational control, |
| G-CSF, IL6, IL-27 | p-Stat1, p-Stat3, p-Stat5 | JAK/STAT pathways |
| | | Growth & Survival Pathways |

Three signatures were identified in AML patient cell samples based on DNA damage response, as measured by p-H2AX levels and p-CHK2 levels, and induction of apoptosis, as measured by cleaved PARP, amine aqua staining and forward and side scatter of light

The signature 1 response to Mylotarg in the primary AML cell sample is characterized by induction of the DNA damage response and apoptosis (FIG. 2). Within this sample, 98% of cells are CD33++, among which approximately 60% are found to have an induced DNA damage response after 6 hours of exposure to Mylotarg, as measured by p-CHK2 and p-H2AX. This sample also exhibits an apoptotic response after 24 and 48 hours, with 22% of live myloid cells becoming PARP+, 3% death by 24 hours, and 20% death by 48 hours, as measured by FSC and viability dyes.

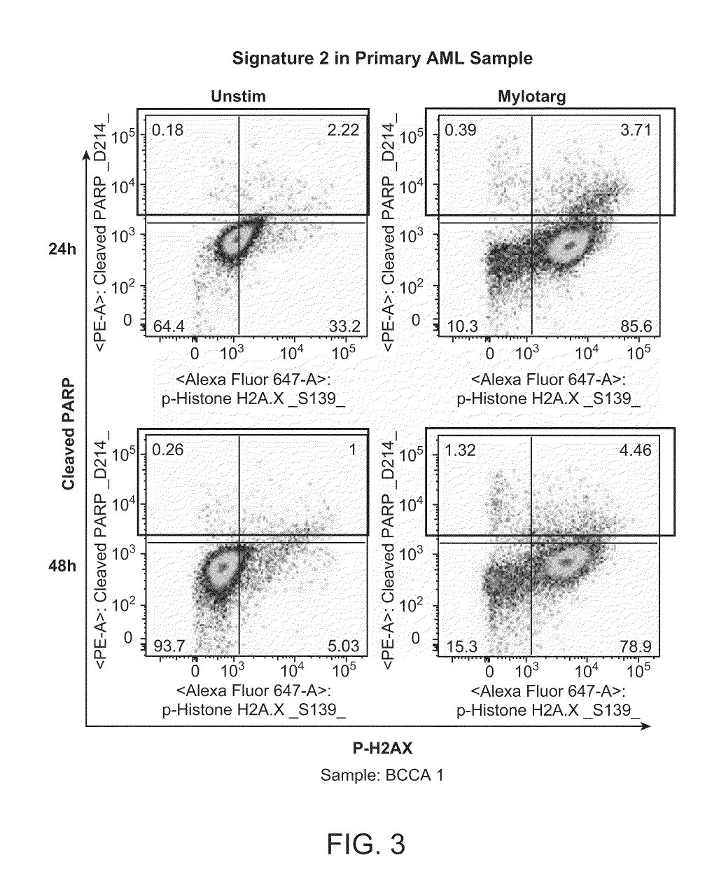

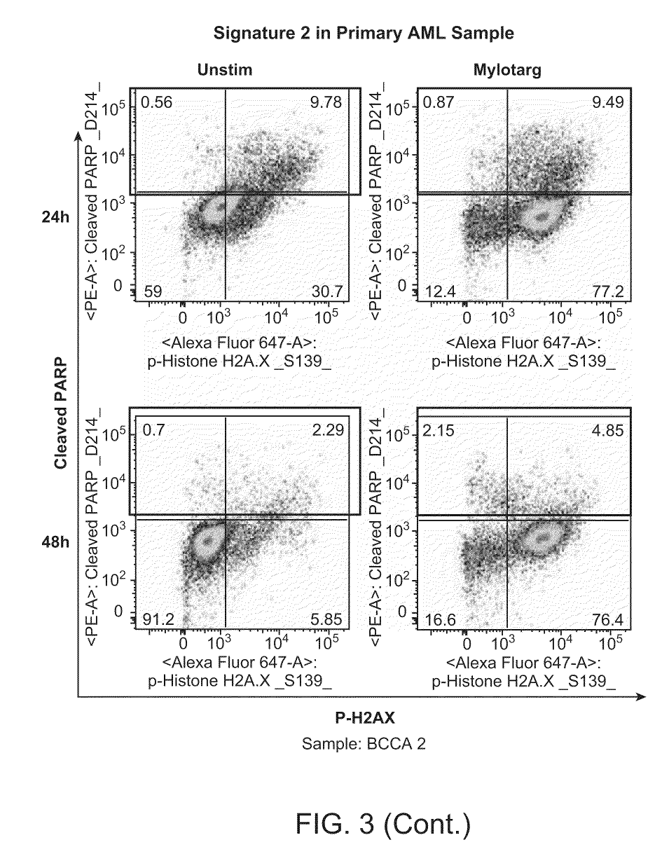

The signature 2 response to Mylotarg in the primary AML cell sample is characterized by induction of the DNA damage response but no apoptosis (FIG. 3). Within this sample, 96% of cells are CD33++, among which approximately 80% are found to have an induced DNA damage response after 6 hours of exposure to Mylotarg, as measured by p-CHK2 and p-H2AX. This sample exhibits no apoptotic response after 24 or 48 hours, with 4% of live myeloid cells becoming PARP+, 0.7% death by 24 hours, and 10% death by 48 hours, as measured by FSC and viability dyes.

The signature 3 response to Mylotarg in the primary AML cell sample is characterized by a lack of a DNA damage response and a lack of apoptosis (FIG. 4). Within this sample, 70% of cells are CD33+, among which approximately 15% are found to have an induced DNA damage response after 6 hours of exposure to Mylotarg, as measured by p-CHK2 and p-H2AX. This sample fails to exhibit much of an apoptotic response, with 8% of live myeloid cells becoming PARP+, 0.7% death by 24 hours, and 8% death by 48 hours, as measured by FSC and viability dyes.

Mylotarg sensitivity in the examined cell lines is also correlated with increased levels of CD33 expression, while resistance correlates with MDR-1 expression. The contribution of MDR-1 to Mylotarg resistance is further illustrated by measuring the efflux of a detectable fluorescent MDR-1 substrate (DIOC2(3)) in the presence or absence of the transporter inhibitor PSC-833. In the Mylotarg sensitive cell line U937, the inhibitor has no effect on intracellular levels of DIOC2(3), but in the Mylotarg resistant cell line KG-1, the addition of inhibitor results in a significant increase in intracellular reporter, indicating a significant level of activity of MDR-1 in the absence of PSC-833 (FIG. 5).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

We claim:

1. A method of classification, diagnosis, prognosis and/or prediction of an outcome of a condition in an individual, said method comprising:
(i) contacting a cell population from said individual with a DNA damage or apoptosis inducing therapeutic agent, wherein said cell population comprises one or more cells associated with a condition, and wherein said agent is used to treat said condition;
(ii) determining an activation level of at least one activatable element within a DNA damage pathway and an activation level of at least one activatable element within an apoptosis pathway in one or more cells from said cell population; and
(iii) making a decision regarding classification, diagnosis, prognosis and/or prediction of an outcome of said condition in said individual, wherein said decision is based on said activation levels of said at least one activatable element within said DNA damage pathway and said at least one activatable element within said apoptosis pathway.

2. The method of claim 1, wherein said cell population is a hematopoietic cell population.

3. The method of claim 1, wherein said cell population might comprises one or more cells that might be resistant to said DNA damage or apoptosis inducing therapeutic agent.

4. The method of claim 1, wherein said condition is acute leukemia, myelodysplastic syndrome or myeloproliferative neoplasms.

5. The method of claim 1, wherein said DNA damage or apoptosis inducing therapeutic agent is selected from the group consisting of Staurosporine, Etoposide, Mylotarg, Daunorubicin, Idarubicin and analogs (idarubicin, epirubicin), Ara-C, Vidaza, Mitoxantrone, Clofarabine, Cladribine, Dacogen, HydroxyUrea, Zolinza, Rituxan, Fludarabine, Floxuridine, 5-FU, Gemcitabine, Cisplatin, ifosfamide, alkylating agents, nucleoside analogs, mechlorethamine and other nitrogen mustards, mercaptopurine, teniposide, Thioguanine, topotecan, and troxacitabine.

6. The method of claim 1, wherein said at least one activatable element within said DNA Damage pathway is selected from the group consisting of p-Chk1, p-Chk2, p-53, p-ATM, and p-H2AX.

7. The method of claim 1, wherein said activatable element within said apoptosis pathway is selected from the group consisting of Cleaved PARP, Cleaved Caspase 3, Cleaved Caspase 8, BAX, Bak, and Cytochrome C.

8. The method of claim 1, further comprising determining a functional state of said apoptosis pathway or said DNA damage pathway, wherein said functional state is based on said activation levels of said activatable elements.

9. The method of claim 8, further comprising a prediction of the outcome of said condition to treatment with said DNA damage or apoptosis inducing therapeutic agent, wherein said individual is predicted to respond to treatment if both the apoptosis and DNA damage pathways are functional the individual can respond to treatment, wherein said individual is predicted to respond to treatment if the DNA damage pathway is not functional but the apoptosis pathway is functional, wherein said individual is predicted not to respond to treatment if the DNA damage pathway is functional but the apoptosis pathway is not functional, and wherein said individual is predicted not to respond to treatment if both the apoptosis and DNA damage pathways are not functional.

10. The method of claim 9, wherein said determination guides selection of a therapeutic treatment for said individual.

11. The method of claim 1, further comprising determining the activation level of at least one activatable element within a cell cycle pathway.

12. The method of claim 11, wherein said at least one activatable element within a cell cycle pathway is selected from the group consisting of Cdc25, p-p53, cCdk1, CyclinB1, p16, p21, p-Histone H3 and Gadd45.

13. The method of claim 1, further comprising contacting said cell population comprising one or more cells associated with said condition from an individual with an additional modulator and characterizing an additional pathway by determining the activation level of at least one activatable element within said additional pathway.

14. The method of claim 13, wherein said additional pathway is selected from the group consisting of drug conversion into an active agent, internal cellular pH, redox potential environment, phosphorylation state of ITIM; drug activation; and signaling pathways.

15. The method of claim 13, wherein said additional pathway is selected from the group consisting of Jak/Stat, PI3K/Akt, and MAPK pathways.

16. The method of claim 15, wherein the activatable element within the PI3K/AKT or MAPK pathways is selected from the group consisting of Akt, p-ERK, p-SyK, p38 and pS6 and the modulator is selected from the group consisting of FLT3L, SCF, G-CSF, GM-CSF, SCF, SDF1a, LPS, PMA, and Thapsigargin.

17. The method of claim 15, wherein the activatable element within the STAT pathway is selected from the group consisting of p-Stat3, p-Stat5, p-Stat1, and p-Stat6 and the modulator is selected from the group consisting of IFNg, IFNa, IL-27, IL-3, IL-6, IL-10, GM-CSF and G-CSF.

18. The method of claim 1, wherein said method further comprises determining the presence or absence of one or more cell surface markers, intracellular markers, or combination thereof.

19. The method of claim 18, wherein said cell surface markers and said intracellular markers are independently selected from the group consisting of proteins, carbohydrates, lipids, nucleic acids and metabolites.

20. The method of claim 18, wherein said determining of the presence or absence of one or more cell surface markers or intracellular markers comprises determining the presence or absence of an epitope in both activated and non-activated forms of said cell surface markers or said intracellular markers.

21. The method of claim 18, wherein the classification, diagnosis, prognosis and/or prediction of outcome of said condition in an individual is based on both the activation levels of said activatable element and the presence or absence of said one or more cell surface markers, intracellular markers, or combination thereof.

22. The method of claim 1 wherein said activation level is determined by a process comprising the binding of a binding element which is specific to a particular activation state of the particular activatable element.

23. The method of claim 22, wherein said binding element comprises an antibody, recombinant protein, or fluorescent dye.

24. The method of claim 1, wherein the step of determining the activation level comprises the use of flow cytometry, immunofluorescence, confocal microscopy, immunohistochemistry, immunoelectronmicroscopy, nucleic acid amplification, gene array, protein array, mass spectrometry, patch clamp, 2-dimensional gel electrophoresis, differential display gel electrophoresis, microsphere-based multiplex protein assays, ELISA, and label-free cellular assays to determine the activation level of one or more intracellular activatable element in single cells.

25. A method of classification, diagnosis, prognosis and/or prediction of an outcome of a condition in an individual, said method comprising:
(i) subjecting a cell population from said individual to a therapeutic agent, wherein said therapeutic agent is used to treat cancer, and wherein said cell population comprises one or more cells associated with a condition;
(ii) determining an activation level of at least one activatable element within a first pathway and an activation level of at least one activatable element within a second pathway in one or more cells from said cell population;
(iii) determining the expression and/or function of a drug transporter in the said cells or separate cells from said cell population not subjected to said therapeutic agent; and
(iv) making a decision regarding classification, diagnosis, prognosis of and/or prediction of an outcome of said condition in said individual, wherein said decision is based on said the activation levels of said at least one activatable element within said first pathway, the activation level of said at least one activatable element within said second pathway and said expression and/or function of said drug transporter.

26. The method of claim 25, wherein alternatively step (iii) comprises determining the effect of inhibiting a drug transporter on a response to said therapeutic agent in said cell population.

27. The method of claim 25, wherein said cell population is a hematopoietic cell population.

28. The method of claim 25, wherein said cell population might comprises one or more cells that might be resistant to said therapeutic agent.

29. The method of claim 25, wherein said condition is acute leukemia, myelodysplastic syndrome or myeloproliferative neoplasms.

30. The method of claim 25, wherein said therapeutic agent used to treat cancer is selected from the group consisting of a DNA damaging agent, an apoptosis inducing agent a drug transporter substrate.

31. The method of claim 30, wherein said DNA damaging or apoptosis inducing agent is selected from the group consisting of Staurosporine, Etoposide, Mylotarg, Daunorubicin, Idarubicin and analogs (idarubicin, epirubicin), Ara-C, Vidaza, Mitoxantrone, Clofarabine, Cladribine, Dacogen, HydroxyUrea, Zolinza, Rituxan, Fludarabine, Floxuridine, 5-FU, Gemcitabine, Cisplatin, ifosfamide, alkylating agents, nucleoside analogs, mechlorethamine and other nitrogen mustards, mercaptopurine, teniposide, Thioguanine, topotecan, and troxacitabine.

32. The method of claim 25, wherein said drug transporter is selected from the group consisting of P-glycoprotein (MDR1), MDR-associated protein and breast cancer resistance protein.

33. The method of claim 25 wherein said first pathway or said second pathway is a DNA damage pathway.

34. The method of claim 33, wherein said at least one activatable element within said DNA damage pathway is selected from the group consisting of p-Chk1, p-Chk2, p-p53, p-ATM, and p-H2AX.

35. The method of claim 25, wherein said first pathway or said second pathway is an apoptosis pathway.

36. The method of claim 25, wherein said activatable element within said apoptosis pathway is selected from the group consisting of Cleaved PARP, Cleaved Caspase 3, Cleaved Caspase 8, BAX, Bak and Cytochrome C.

37. The method of claim 25 wherein said first pathway is a DNA damage pathway and said second pathway is as apoptosis pathway.

38. The method of claim 37, further comprising determining a functional state of said apoptosis pathway or said DNA damage pathway, wherein said functional state is based on said activation levels of said activatable elements.

39. The method of claim 38, further comprising a prediction of the outcome of said condition to treatment with said therapeutic agent, wherein said individual is predicted to respond to treatment if both the apoptosis and DNA damage pathways are functional the individual can respond to treatment, wherein said individual is predicted to respond to treatment if the DNA damage pathway is not functional but the apoptosis pathway is functional, wherein said individual is predicted not to respond to treatment if the DNA damage pathway is functional but the apoptosis pathway is not functional, and wherein said individual is predicted not to respond to treatment if both the apoptosis and DNA damage pathways are not functional.

40. The method of claim 39, wherein said determination guides selection of a therapeutic treatment for said individual.

41. The method of claim 37, further comprising determining the activation level of at least one activatable element within a cell cycle pathway.

42. The method of claim 41, wherein said at least one activatable element within a cell cycle pathway is selected from the group consisting of Cdc25, p-p53, cCdk1, CyclinB1, p16, p21, p-Histone H3 and Gadd45.

43. The method of claim 37, further comprising contacting said cell population comprising one or more cells associated with said condition from said individual with an additional modulator and characterizing an additional pathway by determining the activation level of at least one activatable element within said additional pathway.

44. The method of claim 43, wherein said additional pathway is selected from the group consisting of drug conversion into an active agent, internal cellular pH, redox potential environment, phosphorylation state of ITIM; drug activation; and signaling pathways.

45. The method of claim 43, wherein said additional pathway is selected from the group consisting of Jak/Stat, PI3K/Akt, and MAPK pathways.

46. The method of claim 45, wherein the activatable element within the PI3K/AKT or MAPK pathways is selected from the group consisting of p-Akt, p-ERK, p38 and pS6 and the modulator is selected from the group consisting of FLT3L, SCF, G-CSF, SCF, GM-CSF, SDF1a, LPS, PMA, and Thapsigargin.

47. The method of claim 45, wherein the activatable element within the STAT pathway is selected from the group consisting of p-Stat3, p-Stat5, p-Stat1, and p-Stat6 and the modulator is selected from the group consisting of IFNg, IFNa, IL-27, IL-3, IL-6, IL-10, GM-CSF and G-CSF.

48. The method of claim 25, wherein said method further comprises determining the presence or absence of one or more cell surface markers, intracellular markers, or combination thereof.

48. The method of claim 25, wherein said cell surface markers and said intracellular markers are independently selected from the group consisting of proteins, carbohydrates, lipids, nucleic acids and metabolites.

50. The method of claim 48, wherein said determining of the presence or absence of one or more cell surface markers or intracellular markers comprises determining the presence or absence of an epitope in both activated and non-activated forms of said cell surface markers or said intracellular markers.

51. The method of claim 48, wherein the classification, diagnosis, prognosis of and/or prediction of outcome of said condition in an individual is based on both the activation levels of said activatable element and the presence or absence of said one or more cell surface markers, intracellular markers, or combination thereof.

52. The method of claim 25 wherein said activation level is determined by a process comprising the binding of a binding element which is specific to a particular activation state of the particular activatable element.

53. The method of claim 52, wherein said binding element comprises an antibody, recombinant protein, or fluorescent dye.

54. The method of claim 35, wherein the step of determining the activation level comprises the use of flow cytometry, immunofluorescence, confocal microscopy, immunohistochemistry, immunoelectronmicroscopy, nucleic acid amplification, gene array, protein array, mass spectrometry, patch clamp, 2-dimensional gel electrophoresis, differential display gel electrophoresis, microsphere-based multiplex protein assays, ELISA, and label-free cellular assays to determine the activation level of one or more intracellular activatable element in single cells.

55. A method of classification, diagnosis, prognosis and/or prediction of an outcome of AML in an individual, said method comprising:
(i) providing a population of cells comprising AML cells from an individual;
(ii) contacting the cells with therapeutic agent comprising an antibody conjugated to a toxin;
(ii) characterizing in individual cells at least three pathways selected from the group consisting of drug conversion into an active agent, cellular redox potential, signaling pathways, DNA damage pathway and apoptosis pathways, wherein said pathways are characterized by determining the activation level of at least one activatable element within said at least three pathways; and
(iv) correlating the classification, diagnosis, prognosis and/or prediction of an outcome of AML in said individual to the characterization of said at least three pathways.

56. The method of claim 55, further comprising determining drug binding, a drug transported expression and/or function in said a population of cells comprising AML cells from an individual.

57. The method of claim 55, wherein said drug transporter is selected from the group consisting of P-glycoprotein (MDR1), MDR-associated protein and breast cancer resistance protein.

58. The method of claim 55 wherein said therapeutic agent comprising an antibody conjugated to a toxin is Mylotarg.

59. The method of claim 58 further comprising determining expression of CD33 and/or ITIM phosphorylation.
